(19) United States
(12) Patent Application Publication
(10) Pub. No.: US 2025/0154140 A1
(43) Pub. Date: May 15, 2025

(54) ISOQUINOLINE DERIVATIVES AS INHIBITORS OF BAX AND/OR BAK, COMPOSITIONS AND USES THEREOF

(71) Applicants: Sunnybrook Research Institute, Toronto (CA); Ontario Institute for Cancer Research (OICR), Toronto (CA)

(72) Inventors: David Andrews, Toronto (CA); Justin Kale, Mississauga (CA); Justin Pogmore, Toronto (CA); Rima Al-Awar, Toronto (CA); Methvin Isaac, Brampton (CA); Gennady Poda, Toronto (CA); David Uehling, Toronto (CA); Tao Xin, Woodbridge (CA); Joshua Barry Cohen, Canton, MA (US); Bridget McCarthy Cole, Quincy, MA (US)

(73) Assignees: Sunnybrook Research Institute, Toronto, ON (CA); Ontario Institute for Cancer Research (OICR), Toronto, ON (CA)

(21) Appl. No.: 18/838,651

(22) PCT Filed: Feb. 17, 2023
(86) PCT No.: PCT/CA2023/050214

§ 371 Date: Aug. 15, 2024

Related U.S. Application Data
(60) Provisional application No. 63/311,088, filed on Feb. 17, 2022.

(51) Int. Cl. C07D 413/12; A61K 31/4725; A61K 31/496; A61K 31/5377; C07D 401/12

(52) U.S. Cl. CPC C07D 413/12; A61K 31/4725; A61K 31/496; A61K 31/5377; C07D 401/12

(57) ABSTRACT

The present application relates to isoquinoline compounds of Formula (I), to processes for their preparation and to compositions comprising them. More particularly, the present application relates to compound of Formula (I) that have activity as inhibitors of Bcl2-associated X protein (BAX) and/or Bcl-2 antagonist killer (BAK), and to their use in the treatment of diseases, disorders or conditions treatable by inhibiting BAX and/or BAK such as neurodegenerative diseases, disorders or conditions.

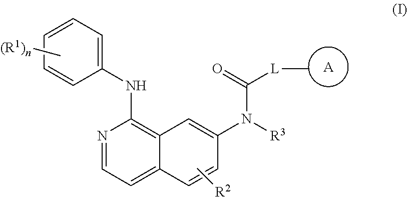

### RELATED APPLICATIONS

The present application claims the benefit of priority of co-pending U.S. provisional patent application No. 63/311,088 filed on Feb. 17, 2022, the contents of which are incorporated herein by reference in their entirety.

### FIELD

The present application relates to isoquinoline compounds, to processes for their preparation, to compositions comprising them, and to their use, for example, in therapy. More particularly, the present application relates to isoquinoline compounds useful in the treatment and/or prevention of diseases, disorders or conditions treatable and/or preventable by inhibiting or blocking the Bcl2-associated X protein (BAX) and/or Bcl-2 antagonist killer (BAK), such as a neurodegenerative disease, disorder or condition.

### BACKGROUND

Apoptosis, a morphologically defined form of programmed cell death, removes superfluous or dysfunctional cells in order to maintain homeostasis (Green and Kroemer, 2004). However, dysregulated apoptosis can contribute to the pathogenesis of neurological disease, inefficient survival of cell-based therapies, and death of healthy cells during cancer chemotherapy. Furthermore, excessive cell death represents a hurdle for biotechnology applications that involve cell manufacturing (Arden and Betenbaugh, 2004; Mergenthaler, Dirnagl and Meisel, 2004; Leber et al., 2010; Octavia et al., 2012; Pang et al., 2017; Afreen et al., 2018; Pemberton, Pogmore and Andrews, 2020).

The Bcl-2 family of proteins control cell death through a complex series of protein-protein interactions that regulate mitochondrial outer membrane permeabilization (MOMP), an event that commits most cells to apoptotic death (Kale et al., 2018). Bcl-2 family proteins contain common sequences known as Bcl-2 homology (BH) motifs that comprise the binding sites and ligands that mediate interactions between the proteins (Aouacheria et al., 2015). The minimum requirement for classification as a Bcl-2 member consists of having a BH3 motif sufficient to bind to multi-BH motif proteins. Deletion of the BH3 motif renders most Bcl-2 members inactive (Leveille et al., 2010, Chi et al., 2020). Proapoptotic “BH3 only” proteins like BID and BIM can bind to and activate multi-BH motif executioner proteins, including Bcl2-associated X protein (BAX), Bcl-2 antagonist killer (BAK), and Bcl-2 ovarian killer (BOK) (Lovell et al. 2008, Fernandez-Marrero et al., 2017, Hockings et al., 2015, Kuwana et al., 2005).

MOMP results from the Bcl-2 family executioner proteins BAX, BAK, and, in some cases BOK, inserting into and oligomerizing within mitochondria outer membranes. Oligomerization of BAX and BAK is initiated by direct binding of BH3-only activator Bcl-2 family members. Anti-apoptotic proteins bind to and thereby inhibit both apoptosis executioner proteins and BH3-only activators (Kim et al., 2009; Llambi et al., 2011; Shamas-Din et al., 2013). MOMP releases cytochrome c and other pro-apoptotic factors from the mitochondrial intermembrane space, triggering an apoptotic signaling cascade and activation of caspases that ultimately dismantle the cell (Tait and Green, 2010; Westphal et al., 2011; Hill, MacKenzie and Harwig, 2015).

Inhibition of BAX and BAK oligomerization with compounds such as DAN004 has been shown to prevent MOMP (Niu et al., 2017). Recently, a variety of other small molecules have been described that activate or inhibit the function of BAX (Pogmore, Uehling and Andrews, 2021). Furthermore, BAl1 was reported to inhibit BAX mediated cell death associated with doxorubicin-induced cardiomyopathy in mice (Amgalan et al., 2020). Despite extensive efforts to develop drugs that directly modulate BAX, no BAX inhibitors have successfully advanced into the clinic. Therefore, there remains a need for BAX and/or BAK inhibitors with clinical potential.

### SUMMARY

Accordingly, the present invention includes a compound of Formula I, or a pharmaceutically acceptable salt, solvate and/or prodrug thereof:

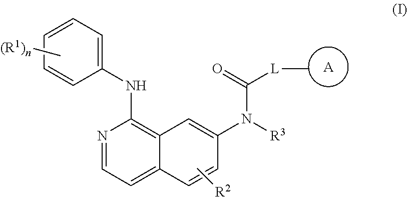

wherein
each R¹ is independently selected from halo, CN, C₁₋₄alkyl, C₁₋₄haloalkyl, OC₁₋₄alkyl and OC₁₋₄haloalkyl;
R² is absent, or R² is selected from halo, C₁₋₄alkyl, C₁₋₄haloalkyl, OC₁₋₄alkyl and OC₁₋₄haloalkyl;
R³ is selected from H and C₁₋₄alkyl;
L is selected from C₁₋₄alkylene, C₂₋₄alkenylene and C₂₋₄alkynylene;
A is C₃₋₈heterocycloalkyl including at least one ring heteromoiety selected from N, NH and N(C₁₋₄alkyl), and optionally substituted with one or two substituents selected from OH, halo, C₁₋₂alkyl, C₁₋₂haloalkyl, OC₁₋₂alkyl and OC₁₋₂haloalkyl; and
n is selected from 0, 1, 2 and 3.

The present application also includes a pharmaceutical composition comprising one or more compounds of the application and a pharmaceutically acceptable carrier.

The present application also includes further includes a method of inhibiting Bcl2-associated X protein (BAX) and/or Bcl-2 antagonist killer (BAK) in a cell, either in a biological sample or in a patient, comprising administering an effective amount of one or more compounds of the application to the cell.

The present application also includes method of treating or preventing BAX and/or BAK mediated cell death in a cell either in a biological sample or in a patient comprising administering an effective amount of one or more compounds the application to the cell.

The present application includes a method of inhibiting MOMP in a cell, either in a biological sample or in a patient comprising administering an effective amount of one or more compounds of any one of the applications to the cell.

The present application includes method for inhibiting BAX and BAK oligomerization in a cell, either in a biological sample or in a patient comprising administering an effective amount of one or more compounds of the application to the cell.

The present application also includes a method of treating a disease, disorder or condition that is treatable by inhibiting Bcl2-associated X protein (BAX) and/or Bcl-2 antagonist killer (BAK) comprising administering a therapeutically effective amount of one or more compounds of the application to a subject in need thereof.

In some embodiments, the disease, disorder or condition that is treatable by inhibiting BAX and/or BAK is a neurodegenerative disease, disorder or condition, neuronal damage associated with ischemia, is cardiomyopathy induced by a chemotherapeutic agent and/or donor hematopoietic stem and progenitor cells (HSPCs) cell death.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### BRIEF DESCRIPTION OF THE DRAWINGS

The present application will be described in greater detail with reference to the attached drawings and Tables in which:

FIG. 1 shows that chemical shift perturbations and computational modeling reveal a putative binding site for dacomitinib on BAX. A. ¹H, ¹⁵N HSQC of 200 uM BAX+1.5% DMSO (dark bars) vs. 200 uM BAX+400 uM dacomitinib (light bars). B. Chemical shift perturbations quantified using CCPNMRv3 software identify localized changes in chemical environment of residues in the presence of dacomitinib. Residues with chemical shifts over the significance threshold or >1.5 times the significance threshold are those over the lower and upper dotted lines respectively. C. Model of BAX (PDB: 1F16) mapping residues undergoing significant CSP on the ribbon structure (structure to the left) and surface representation (structure to the right using the same shading indicators from the CSP histogram in B.

FIG. 2 are graphs showing: A. Chemical shift perturbations quantified using CCPNMRv3 software identify localized changes in chemical environment of residues in the presence of exemplary compound I-1. Residues with chemical shifts over the significance threshold or >1.5 times the significance threshold are those over the lower and upper dotted lines, respectively. B. BAX mutant V83W, L120W reduced inhibition of BAX by dacomitinib. 100 nM of BAX or V83W L120W BAX and 20 nM of cBID were incubated with ANTS/DPX liposomes and 20 uM dacomitinib as indicated. Shown are the means of 3 independent experiments in duplicate.

FIG. 3 shows that dacomitinib and exemplary compound I-1 inhibit BAX and BAK mediated permeabilization of mitochondrial membranes independent of activating signal. Mitochondria isolated from mouse liver were used for these assays because in this tissue BAX is cytoplasmic and therefore absent in heavy membrane isolates. Bcl-XL serves as a positive control for complete inhibition of BAX. Mitochondria only control for non-specific leakage of isolated mitochondria. A. Dacomitinib and exemplary compound I-1 attenuate cBID activated BAX mediated permeabilization of isolated mitochondria. Mitochondria isolated from BAK^{-/-} mouse liver were incubated with 10 nM BAX and 2 nM cBID and of the concentration of each inhibitor as indicated. B. Dacomitinib and exemplary compound I-1 attenuate BIM activated BAK mediated permeabilization of isolated mitochondria. BIM (2.5 nM) was incubated with mitochondria isolated from WT mouse liver and the indicated concentration of each inhibitor. Mitochondria were incubated at 37° C. for 30 minutes before pelleting and measuring the fluorescence of SMAC-mCherry in the supernatant (released from mitochondria) and resuspended pellet fractions.

FIG. 4 shows that kinase and BAX inhibitory activities can be separated by chemically modifying the kinase binding hinge region of dacomitinib. A. Kinase panel assessing exemplary compound I-1 activity at 1 uM, performed by Eurofins. Percent kinase inhibition is calculated by subtracting the activity remaining in the presence of the compound from the activity of the kinase with DMSO instead of exemplary compound I-1. Negative values reflect differences in the uninhibited activities of some kinases and are interpreted as observing no inhibition. B. Liposome membrane permeabilization assay using 20 nM cBID and 100 nM BAX against a titration of dacomitinib and exemplary compound I-1. Each point represents one of two technical replicates.

FIG. 5: shows that cell death induced by actinomycin D depends on the pro-apoptotic activity of BAX and BAK. A. BMK cell death induced by actinomycin D is mediated by both BAX and BAK. BMK cells of the indicated genotypes were exposed to the indicated concentrations of actinomycin D for 20-24 hours and then cell death was assessed by confocal microscopy measurements of TMRE, Annexin V and Hoechst staining. Data from three technical replicates are shown fit to a one phase association curve. B. Baseline cell death in the presence of dacomitinib and exemplary compound I-1 are equivalent in BMK cells independent of the expression of Bax and Bak. Actinomycin D induced BAX and BAK mediated cell death as cell death is highest for cells expressing both BAX and BAK and least for cells that do not express either pro-apoptotic protein (BAX−/− BAK−/−).

FIG. 6 shows concentration dependent inhibition of spontaneous and actinomycin D induced death of wild-type BMK (BMK-wt) cells by dacomitinib. 0 nM actinomycin D indicates that dacomitinib decreased spontaneous cell death in culture from ˜30% to <10%. The other panels show cell death due to 3.125 nM, 6.25 nM and 12.5 nM concentrations of actinomycin D (indicated above the panels) was reduced by the addition of the indicated concentrations of dacomitinib. The bars indicate the mean values for four technical replicates indicated by individual symbols. Cells were classified as dead using a linear classifier to analyze micrographs of individual cells stained with the mitochondrial potential sensitive dye TMRE, the nuclear stain Hoescht and fluorescent Annexin V.

FIG. 7 shows concentration dependent inhibition of spontaneous and actinomycin D induced death of wild-type BMK (BMK-wt) cells by the exemplary compound 1-1. OnM actinomycin D indicates effects of I-1 on spontaneous cell death in culture. The other panels show cell death due to 3.125 nM, 6.25 nM and 12.5 nM concentrations of actinomycin D (indicated above the panels) was reduced by the addition of the indicated concentrations of I-1. The bars indicate the mean values for four technical replicates indicated by individual symbols. Cells were classified as dead using a linear classifier to analyze micrographs of individual cells stained with the mitochondrial potential sensitive dye TMRE, the nuclear stain Hoescht and fluorescent Annexin V.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise

The term “compound of the application” or “compound of the present application” and the like as used herein refers to a compound of Formula I and I-A including pharmaceutically acceptable salts, solvates and/or prodrugs thereof.

The term “composition of the application” or “composition of the present application” and the like as used herein refers to a composition comprising one or more compounds the application and at least one additional ingredient.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the application exist as individual salts and hydrates, as well as a combination of, for example, a solvate of a salt of a compound of the application.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process/method steps.

As used herein, the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The present application refers to a number of chemical terms and abbreviations used by those skilled in the art. Nevertheless, definitions of selected terms are provided for clarity and consistency.

The products of the processes of the application may be isolated according to known methods, for example, the compounds may be isolated by evaporation of the solvent, by filtration, centrifugation, chromatography or other suitable method.

One skilled in the art will recognize that where a reaction step of the present application is carried out in a variety of solvents or solvent systems, said reaction step may also be carried out in a mixture of the suitable solvents or solvent systems.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “cell” as used herein refers to a single cell or a plurality of cells and includes a cell either in a cell culture or in a subject.

The term “subject” as used herein includes all members of the animal kingdom including mammals. Thus, the methods and uses of the present application are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with an active ingredient (for example, one or more compounds of the application) to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “prodrug” as used herein means a compound, or salt and/or solvate of a compound, that, after administration, is converted into an active drug.

The term “solvate” as used herein means a compound, or a salt or prodrug of a compound, wherein molecules of a suitable solvent are incorporated in the crystal lattice.

The term “inert organic solvent” as used herein refers to a solvent that is generally considered as non-reactive with the functional groups that are present in the compounds to be combined together in any given reaction so that it does not interfere with or inhibit the desired synthetic transformation. Organic solvents are typically non-polar and dissolve compounds that are nonsoluble in aqueous solutions.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₁₀alkylene means an alkylene group having 1, 2, 3, 4, 5, 6, 7, 8, 9 or 10 carbon atoms. All alkyl groups are optionally fluorosubstituted unless otherwise indicated.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

The term “heterocycloalkyl” as used herein, whether it is used alone or as part of another group, refers to cyclic groups containing at least one non-aromatic ring containing from 3 to 10 atoms in which one or more of the atoms are a heteromoiety selected from O, S, S(O), SO₂, N, NH and N(C₁₋₆alkyl), and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heterocycloalkyl groups are optionally benzofused.

All cyclic groups, including aryl, heteroaryl, heterocycloalkyl and cycloalkyl groups, contain one (i.e. are monocyclic) or more than one ring (i.e. are polycyclic). When a cyclic group contains more than one ring, the rings may be fused, bridged or spirofused.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “haloalkyl” as used herein refers to an alkyl group as defined above in which one or more of the available hydrogen atoms have been replaced with a halogen. Thus, for example, “C₁₋₆ haloalkyl” (or “C₁-C₆ haloalkyl”) refers to a C₁ to C₆ linear or branched alkyl group as defined above with one or more halogen substituents.

The terms “halo” or “halogen” as used herein, whether it is used alone or as part of another group, refers to a halogen atom and includes fluoro, chloro, bromo and iodo.

The term “chloroalkyl” as used herein refers to an haloalkyl group as defined above wherein the halogen atom is chloro.

The term “fluoroalkyl” as used herein refers to an haloalkyl group as defined above wherein the halogen atom is fluoro.

The term “available”, as in “available hydrogen atoms” or “available atoms” refers to atoms that would be known to a person skilled in the art to be capable of replacement by another atom or group.

The term “optionally substituted” as used herein means that the referenced group is unsubstituted or substituted.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of a disease, disorder or condition, stabilized (i.e. not worsening) state of a disease, disorder or condition, preventing spread of a disease, disorder or condition, delay or slowing of a disease, disorder or condition progression, amelioration or palliation of a disease, disorder or condition state, diminishment of the reoccurrence of a disease, disorder or condition, inhibiting or reducing a disease, disorder or condition and remission (whether partial or total), whether detectable or undetectable. “Treating” and “treatment” can also mean prolonging survival as compared to expected survival if not receiving treatment. “Treating” and “treatment” as used herein also include prophylactic treatment.

“Palliating” a disease, disorder or condition means that the extent and/or undesirable clinical manifestations of the disease, disorder or condition are lessened and/or time course of the progression is slowed or lengthened, as compared to not treating the disease, disorder or condition.

The term “prevention” or “prophylaxis”, or synonym thereto, as used herein refers to a reduction in the risk or probability of a subject becoming afflicted with a disease, disorder or condition treatable by inhibition of BAX or manifesting a symptom associated with a disease, disorder or condition treatable by inhibition of BAX.

As used herein, the term “effective amount” or “therapeutically effective amount” means an amount of a compound, or one or more compounds, of the application that is effective, at dosages and for periods of time necessary to achieve the desired result.

The term “disease, disorder or condition treatable by inhibiting Bcl2-associated X protein (BAX) and/or Bcl-2 antagonist killer (BAK)” means that the disease, disorder or condition to be treated is affected by, modulated by and/or has some biological basis, either direct or indirect, that includes BAX and/or BAK activity, in particular, increased BAX activity. These diseases respond favourably when BAX and/or BAK activity associated with the disease, disorder or condition is inhibited by one or more of the compounds or compositions of the application.

The expression “inhibiting Bcl2-associated X protein (BAX) and/or Bcl-2 antagonist killer (BAK)” as used herein refers to inhibiting, blocking and/or disrupting the activity or function of BAX and/or BAK in a cell. The inhibiting, blocking and/or disrupting causes a therapeutic effect in the cell.

By “inhibiting, blocking and/or disrupting” it is meant any detectable inhibition, block and/or disruption in the presence of a compound compared to otherwise the same conditions, except for in the absence in the compound.

The expression “Bcl2-associated X protein (BAX) and/or Bcl-2 antagonist killer (BAK) mediated cell death” means that the cell death is affected by, modulated by and/or has some biological basis, either direct or indirect, that includes BAX and/or BAK activity, in particular, increased BAX activity.

The term “BAX” as used herein refers to Bcl2-associated X protein or any functional mutant or analogous forms thereof.

The term “BAK” as used herein refers Bcl-2 antagonist killer protein or any functional mutant or analogous forms thereof.

The term “apoptosis” as used herein refers to programmed cell death.

The term “MOMP” as used herein refers to mitochondrial outer membrane permeabilization”.

The expression “cardiomyopathy induced by a chemotherapeutic agent” refers to any detectable increase in cardiomyopathy after treatment with a chemotherapeutic agent compared to the cardiomyopathy before treatment with a chemotherapeutic agent.

The expression “increasing the survival of donor hematopoietic stem and progenitor cells (HSPCs) during transplantation” refers to any detectable increase in HSPC survival during transplantation in the presence of a compound of the application compared to otherwise the same conditions, except for in the absence in the compound.

The term “dacomitinib” as used herein refers to the compound having the chemical name: (E)-N-[4-(3-chloro-4-fluoroanilino)-7-methoxyquinazolin-6-yl]-4-piperidin-1-ylbut-2-enamide, and having the chemical formula:

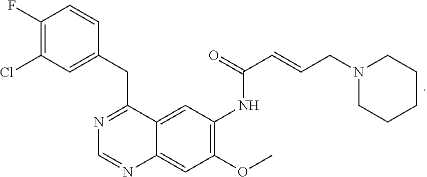 [SMILES: COc1cc2ncnc(Cc3ccc(F)c(Cl)c3)c2cc1NC(=O)/C=C/CN1CCCCC1]

The term “administered” as used herein means administration of a therapeutically effective amount of a compound, or one or more compounds, or a composition of the application to a cell or a subject.

### II. Compounds of the Application

Isoquinoline compounds of the present application were prepared and found to inhibit Bcl2-associated X protein (BAX) and/or Bcl-2 antagonist killer (BAK). These compounds resulted from chemical modifications of the known therapeutic agent, dacomitinib, a known kinase inhibitor, resulting in compounds which the Applicants have surprisingly found to also have both BAX and BAK inhibitor activity. Through systematic modification of the dacomitinib quinazoline core structure, the Applicants have developed the isoquinoline compounds of the application which do not exhibit kinase inhibitor activity, but which have been shown to be effective inhibitors of actinomycin D mediated cell death in cells expressing either BAX or BAK.

Accordingly, the present application includes a compound of Formula I, or a pharmaceutically acceptable salt, solvate and/or prodrug thereof:

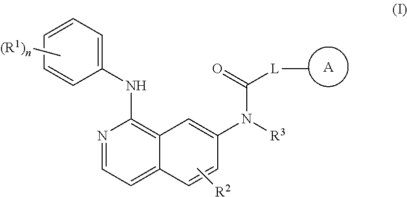

wherein
each R¹ is independently selected from halo, CN, C₁₋₄alkyl, C₁₋₄haloalkyl, OC₁₋₄alkyl and OC₁₋₄haloalkyl;
R² is absent, or R² is selected from halo, C₁₋₄alkyl, C₁₋₄haloalkyl, OC₁₋₄alkyl and OC₁₋₄haloalkyl;
R³ is selected from H and C₁₋₄alkyl;
L is selected from C₁₋₄alkylene, C₂₋₄alkenylene and C₂₋₄alkynylene;
A is C₃₋₈heterocycloalkyl including at least one ring heteromoiety selected from N, NH and N(C₁₋₄alkyl), and optionally substituted with one or two substituents selected from OH, halo, C₁₋₂alkyl, C₁₋₂haloalkyl, OC₁₋₂alkyl and OC₁₋₂haloalkyl; and
n is selected from 0, 1, 2 and 3.

The present application includes a compound of Formula I, or a pharmaceutically acceptable salt, solvate and/or prodrug thereof:

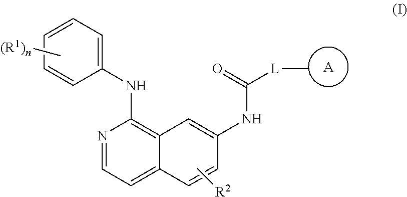

wherein
each R¹ is independently selected from halo, C₁₋₄alkyl and C₁₋₄haloalkyl;
R² is absent, or R² is selected from halo, C₁₋₄alkyl, C₁₋₄haloalkyl, OC₁₋₄alkyl and OC₁₋₄haloalkyl;
L is selected from C₁₋₄alkylene, C₂₋₄alkenylene and C₂₋₄alkynylene;
A is C₃₋₈heterocycloalkyl including at least one ring heteromoiety selected from N, NH and N(C₁₋₄alkyl), and optionally substituted with one or two C₁₋₂alkyl; and
n is an integer selected from 1 to 3.

In some embodiments, each R¹ is independently selected from F, Cl, Br, CN, C₁₋₄alkyl, C₁₋₄haloalkyl, OC₁₋₄alkyl and OC₁₋₄haloalkyl. In some embodiments, each R¹ is independently selected from F, Cl, Br, CN, C₁₋₄alkyl, C₁₋₄fluoroalkyl, C₁₋₄chloroalkyl, OC₁₋₄alkyl, OC₁₋₄fluoroalkyl and OC₁₋₄chloroalkyl. In some embodiments, each R¹ is independently selected from F, Cl, Br, CN, CH₃, CH₂CH₃, CH₂CH₂CH₃, CH(CH₃)₂, CH(CH₃)CH₂CH₃, CH(CH₃)₃, CF₃, CHF₂, CFH₂, CH₂CHF₂, CH₂CF₃, CH₂CFH₂, CCl₃, CH₂CClH₂, CCl₂H, CH₂CCl₂H, CH₂CCl₃, CH₃, OCH₂CH₃, OCH₂CH₂CH₃, OCH(CH₃)₂, OCH(CH₃)CH₂CH₃, OCH(CH₃)₃, OCF₃, OCHF₂, OCH₂CHF₂, OCH₂CF₃, OCH₂CFH₂, OCCl₃, OCH₂CClH₂, OCCl₂H, OCH₂CCl₂H and CH₂CCl₃. In some embodiments, each R¹ is independently selected from F, Cl, Br, CN, CH₃, CH₂CH₃, CH₂CH₂CH₃, CH(CH₃)₂, CF₃, CHF₂, CFH₂, CH₂CHF₂, CH₂CF₃, CH₂CFH₂, CCl₃, CH₂CClH₂, CCl₂H, CH₂CCl₂H, CH₂CCl₃, OCH₃, OCH₂CH₃, OCH₂CH₂CH₃, OCH(CH₃)₂, OCF₃, OCHF₂, OCH₂CHF₂, OCH₂CF₃, OCH₂CFH₂, OCC₃, OCH₂CClH₂, OCCl₂H, OCH₂CCl₂H and OCH₂CCl₃. In some embodiments, each R¹ is independently selected from F, C, Br, CN, CH₃, CH₂CH₃, CH₂CH₂CH₃, CH(CH₃)₂, CF₃, CHF₂, CFH₂, CH₂CHF₂, CH₂CF₃, CH₂CFH₂, CCl₃, CH₂CClH₂, CCl₂H, CH₂CCl₂H, CH₂CCl₃, OCH₃, OCF₃, OCHF₂, OCH₂CHF₂, OCH₂CF₃, OCH₂CFH₂, OCC₃, OCH₂CClH₂, OCCl₂H, OCH₂CCl₂H and OCH₂CCl₃. In some embodiments, each R¹ is independently selected from F, C, CN, CH₃, CH₂CH₃, CH₂CH₂CH₃, CH(CH₃)₂, CF₃, CHF₂, CFH₂, CCl₃, CCl₂H, OCH₃, OCF₃, OCHF₂, OCCl₃ and OCCl₂H. In some embodiments, each R¹ is independently selected from F, Cl, CN, CH₃, CH₂CH₃, CH₂CH₂CH₃, CH(CH₃)₂, CF₃, CHF₂, CCl₃, CCl₂H, OCH₃, OCF₃, OCHF₂, OCCl₃ and OCCl₂H. In some embodiments, each R¹ is independently selected from F, Cl, CN, CH₃, CH₂CH₃, CH₂CH₂CH₃, CH(CH₃)₂, CF₃, CHF₂, CCl₃, OCH₃, OCF₃ and OCHF₂. In some embodiments, each R¹ is independently selected from F, Cl, CN, CH₃, CH₂CH₃, CH₂CH₂CH₃, CH(CH₃)₂, CF₃, OCH₃ and OCF₃. In some embodiments, each R¹ is CN. In some embodiments, each R¹ is independently selected from F, Cl and CF₃. In some embodiments, each R¹ is independently selected from F, Cl, OCH₃ and OCF₃. In some embodiments, each R¹ is independently selected from F, Cl, CH₃, CF₃, CH₂CH₃, CH₂CH₂CH₃ and CH(CH₃)₂. In some embodiments, each R¹ is independently selected from F, Cl, CH₃, CH₂CH₃, CH₂CH₂CH₃ and CH(CH₃)₂. In some embodiments, each R¹ is independently selected from F and Cl.

In some embodiments, each R¹ is independently selected from F, Cl, Br, C₁₋₄alkyl and C₁₋₄haloalkyl. In some embodiments, each R¹ is independently selected from F, Cl, Br, CH₃, CH₂CH₃, CH₂CH₂CH₃, CH(CH₃)₂, CH(CH₃)CH₂CH₃, CH(CH₃)₃, CF₃, CHF₂, CH₂CHF₂, CH₂CF₃, CH₂CFH₂, CCl₃, CH₂CClH₂, CCl₂H, CH₂CCl₂H and CH₂CCl₃. In some embodiments, each R¹ is independently selected from F, Cl, Br, CH₃, CH₂CH₃, CH₂CH₂CH₃, CH(CH₃)₂, CF₃, CHF₂, CH₂CHF₂, CH₂CF₃, CH₂CFH₂, CCl₃, CH₂CClH₂, CCl₂H, CH₂CCl₂H and CH₂CCl₃. In some embodiments, each R¹ is independently selected from F, Cl, Br, CH₃, CF₃, CHF₂, CH₂CHF₂, CH₂CF₃, CH₂CFH₂, CCl₃, CH₂CClH₂, CCl₂H, CH₂CCl₂H and CH₂CCl₃. In some embodiments, each R¹ is independently selected from F, Cl, Br, CH₃, CF₃, CHF₂, CCl₃ and CCl₂H. In some embodiments, each R¹ is independently selected from F, C, Br, CHF₂ and CF₃. In some embodiments, each R¹ is independently selected from F, C, Br, CF₃, and CHF₂. In some embodiments, each R¹ is independently selected from F, C, CF₃, and CHF₂. In some embodiments, each R¹ is independently selected from F, C and CF₃. In some embodiments, each R¹ is independently selected from F and C.

In some embodiments, n is 0. In some embodiments, n is 1 or 2. In some embodiments, n is 1, 2 or 3. In some embodiments, n is 2 or 3. In some embodiments, n is 2.

In some embodiments, R² is selected from F, Br, Cl, C₁₋₄alkyl, C₁₋₄haloalkyl, OC₁₋₄alkyl and OC₁₋₄haloalkyl. In some embodiments, R² is selected from F, C, Br, C₁₋₄alkyl, C₁₋₄fluoroalkyl, C₁₋₄chloroalkyl, OC₁₋₄alkyl, OC₁₋₄fluoroalkyl and OC₁₋₄chloroalkyl. In some embodiments, R² is selected from F, Br, Cl, C₁₋₄alkyl, C₁₋₄haloalkyl, OC₁₋₃alkyl and OC₁₋₃haloalkyl. In some embodiments, R² is selected from F, Br, Cl, C₁₋₃alkyl, C₁₋₃fluoroalkyl, C₁₋₃chloroalkyl, OC₁₋₃alkyl, OC₁₋₃fluoroalkyl and OC₁₋₃chloroalkyl.

In some embodiments R² is selected from F, Cl, Br, CH₃, CH₂CH₃, CH₂CH₂CH₃, CH(CH₃)₂, CF₃, CHF₂, CH₂CHF₂, CH₂CF₃, CH₂CFH₂, CCl₃, CH₂CClH₂, CCl₂H, CH₂CCl₂H, CH₂CCl₃, OCH₃, OCH₂CH₃, OCH₂CH₂CH₃, OCH(CH₃)₂, OCF₃, OCHF₂, OCH₂CHF₂, OCH₂CF₃, OCH₂CFH₂, OCCl₃, OCH₂CClH₂, OCCl₂H, OCH₂CCl₂H and OCH₂CCl₃. In some embodiments, R² is selected from F, Cl, Br, CH₃, CH₂CH₃, CF₃, CHF₂, CCl₃, CCl₂H, OCH₃, OCH₂CH₃, OCH₂CH₂CH₃, OCH(CH₃)₂, OCF₃, OCHF₂, OCH₂CHF₂, OCH₂CF₃, OCH₂CFH₂, OCCl₃, OCH₂CClH₂, OCCl₂H, OCH₂CCl₂H and OCH₂CCl₃. In some embodiments R² is selected from F, Cl, Br, CH₃, CH₂CH₃, CF₃, CHF₂, CCl₃, CCl₂H, CH₂CCl₂H, CH₂CCl₃, OCH₃, OCH₂CH₃, OCF₃, OCHF₂, OCH₂CHF₂, OCH₂CF₃, OCH₂CFH₂, OCCl₃, OCH₂CClH₂, OCCl₂H, OCH₂CCl₂H and OCH₂CCl₃. In some embodiments R² is selected from F, Cl, Br, CH₃, CH₂CH₃, OCH₃, CF₃, CHF₂, CCl₃, CCl₂H, OCF₃, OCHF₂, OCCl₃ and OCCl₂H. In some embodiments R² is selected from F, Cl, CH₃, CF₃, CHF₂, CCl₃, CCl₂H, OCH₃, OCH₂CH₃, OCH₂CH₂CH₃, OCH(CH₃)₂, OCF₃, OCHF₂, OCCl₃ and OCCl₂H. In some embodiments R² is selected from CF₃, CHF₂, CCl₃, CCl₂H, OCH₃, OCF₃, OCHF₂, OCCl₃ and OCCl₂H. In some embodiments, R² is selected from F, Cl, CH₃, CF₃, OCH₃, OCH₂CH₃, OCH₂CH₂CH₃, OCH(CH₃)₂, OCF₃ and OCHF₂. In some embodiments, R² is CH₃. In some embodiments R² is selected from OCH₃, OCF₃, OCHF₂, OCCl₃ and OCCl₂H. In some embodiments R² is selected from OCH₃, OCH₂CH₃, OCH₂CH₂CH₃, OCH(CH₃)₂, OCF₃ and OCHF₂. In some embodiments R² is selected from F and Cl. In some embodiments R² is selected from OCH₃, OCF₃ and OCCl₃. In some embodiments R² is OCH₃.

In some embodiments, R² is absent.

In some embodiments, R³ is selected from H and C₁₋₃alkyl. In some embodiments, R³ is selected from H, CH₃, CH₂CH₃, CH₂CH₂CH₃ and CH(CH₃)₂. In some embodiments, R³ is selected from H and CH₃. In some embodiments, R³ is H.

In some embodiments, L is selected from C₁₋₄alkylene and C₂₋₄alkenylene. In some embodiments, L is C₁₋₄alkylene. In some embodiments, L is selected from CH₂CH₂CH₂CH₂, CH₂CH₂CH₂, CH₂CH₂ and CH₂. In some embodiments, L is selected from CH₂CH₂CH₂CH₂, CH₂CH₂CH₂, and CH₂CH₂. In some embodiments, L is selected from CH₂CH₂CH₂ (C₃alkylene) and CH₂CH₂ (C₂alkylene). In some embodiments, L is CH₂CH₂. In some embodiments, L is CH₂CH₂CH₂.

In some embodiments, L is C₂₋₄alkenylene. In some embodiments, L is selected from CHCHCH₂CH₂, CHCHCH₂, CHCH, CH₂CHCHCH₂ and CH₂CHCH. In some embodiments, L is selected from CHCHCH₂CH₂, CHCHCH₂, CH₂CHCHCH₂ and CH₂CHCH. In some embodiments, L is selected from CHCHCH₂, and CH₂CHCH.

In some embodiments, A is C₃₋₈heterocycloalkyl including at least one ring heteromoiety selected from N, NH and N(C₁₋₄alkyl), and optionally substituted with one or two substituents selected from OH, F, Cl, C₁₋₂alkyl, C₁₋₂fluoroalkyl, C₁₋₂chloroalkyl, OC₁₋₂alkyl, OC₁₋₂fluoroalkyl and OC₁₋₂chloroalkyl. In some embodiments, A is C₃₋₆heterocycloalkyl including at least one ring heteromoiety selected from N, NH and N(C₁₋₄alkyl), and optionally substituted with one or two substituents selected from OH, F, C, C₁₋₂alkyl, C₁₋₂fluoroalkyl, C₁₋₂chloroalkyl, OC₁₋₂alkyl, OC₁₋₂fluoroalkyl and OC₁₋₂chloroalkyl.

In some embodiments, A is C₃₋₆heterocycloalkyl including at least one ring heteromoiety selected from N, NH and N(C₁₋₄alkyl), and optionally substituted with one or two substituents selected from OH, F, C, C₁₋₂alkyl, CF₃, CHF₂, CCl₃, CCl₂H, OCH₃, OCH₂CH₃, OCF₃, OCHF₂, OCH₂CHF₂, OCH₂CF₃, OCH₂CFH₂, OCC₃, OCH₂CClH₂, OCCl₂H, OCH₂CCl₂H and OCH₂CCl₃.

In some embodiments, the C₃₋₆heterocycloalkyl including at least one ring heteromoiety selected from N, NH and N is selected from aziridinyl, azetidinyl, pyrrolidinyl, morpholinyl, piperazinyl, and piperidinyl and A is selected from aziridinyl, azetidinyl, pyrrolidinyl, morpholinyl, piperazinyl, and piperidinyl, optionally substituted with one or two OH, F, Cl, C₁₋₂alkyl, CF₃, CHF₂, CCl₃, CCl₂H, OCH₃, OCH₂CH₃, OCF₃, OCHF₂, OCH₂CHF₂, OCH₂CF₃, OCH₂CFH₂, OCC₃, OCH₂CClH₂, OCCl₂H, OCH₂CCl₂H and OCH₂CCl₃. In some embodiments, A is selected from aziridinyl, azetidinyl, pyrrolidinyl, morpholinyl, piperazinyl, and piperidinyl and optionally substituted with one or two C₁₋₂alkyl. In some embodiments, A is selected from pyrrolidinyl, morpholinyl, piperazinyl and piperidinyl and optionally substituted with one or two substituents selected from OH, F, Cl, C₁₋₂alkyl, CF₃, CHF₂, OCH₃, OCH₂CH₃, OCF₃, OCHF₂, OCH₂CHF₂, OCH₂CF₃ and OCH₂CFH₂. In some embodiments, A is selected from pyrrolidinyl, morpholinyl, piperazinyl and piperidinyl and optionally substituted with one or two C₁₋₂alkyl. In some embodiments, A is selected from pyrrolidinyl, morpholinyl, piperazinyl and piperidinyl and optionally substituted with one or two substituents selected from OH, F, Cl, CH₃, CH₂CH₃, CF₃, CHF₂, OCH₃, OCH₂CH₃, OCF₃, OCHF₂, OCH₂CHF₂, OCH₂CF₃ and OCH₂CFH₂. In some embodiments, A is selected from pyrrolidinyl, morpholinyl, piperazinyl and piperidinyl and optionally substituted with one or two substituents selected from OH, F, Cl, CH₃, CF₃, CHF₂, OCH₃, OCF₃ and OCHF₂. In some embodiments, A is selected from pyrrolidinyl, morpholinyl, piperazinyl and piperidinyl and optionally substituted with one or two substituents selected from F, Cl, CH₃, CF₃, OCH₃ and OCF₃. In some embodiments, A is selected from pyrrolidinyl, morpholinyl, piperazinyl and piperidinyl and optionally substituted with one or two CH₃. In some embodiments, A is selected from pyrrolidinyl, morpholinyl, piperazinyl and piperidinyl and optionally substituted with one or two CH₃.

In some embodiments, A is selected from pyrrolidinyl, morpholinyl, piperazinyl and piperidinyl and optionally substituted with one or two F. In some embodiments, A is selected from pyrrolidinyl, morpholinyl, piperazinyl and piperidinyl and optionally substituted with one or two F.

In some embodiments, A is selected from pyrrolidinyl, morpholinyl, piperazinyl and piperidinyl and optionally substituted with one or two substituents selected from OCH₃ and OCH₃. In some embodiments, A is selected from pyrrolidinyl, morpholinyl, piperazinyl and piperidinyl and optionally substituted with one or two with one or two substituents selected from OCH₃ and OCH₃.

In some embodiments, A is selected from pyrrolidinyl and piperazinyl and piperidinyl, and optionally substituted with one or two C₁₋₂alkyl. In some embodiments, A is selected from pyrrolidinyl and piperazinyl and piperidinyl, and optionally substituted with one or two C₁₋₂alkyl. In some embodiments, A is piperidinyl and optionally substituted with one or two C₁₋₂alkyl.

In some embodiments, A is unsubstituted. In some embodiments, A is selected from pyrrolidinyl, morpholinyl, piperazinyl and piperidinyl. In some embodiments, A is selected from pyrrolidinyl, piperazinyl and piperidinyl. In some embodiments, A is piperidinyl.

In some embodiments, n is 2 and each R¹ is independently selected from F, Cl, CN, CH₃, CH₂CH₃, CH₂CH₂CH₃, CH(CH₃)₂, CF₃, OCH₃ and OCF₃. In some embodiments, n is 2 and each R¹ is independently selected from F, Cl, Br, CH₃, CF₃, CHF₂, CCl₃ and CCl₂H. In some embodiments, n is 2 and each R¹ is independently selected from F, Cl, Br, CHF₂ and CF₃. In some embodiments, n is 2 and each R¹ is independently selected from F, Cl, CF₃ and CHF₂. In some embodiments, n is 2 and each R¹ is independently selected from F, Cl and CF₃. In some embodiments, n is 2 and each R¹ is independently selected from F and C.

In some embodiments, n is 2 or 3, each R¹ is independently selected from F and Cl and R² is selected from F, Cl, CH₃, CF₃, OCH₃, OCH₂CH₃, OCH₂CH₂CH₃, OCH(CH₃)₂, OCF₃ and OCHF₂. In some embodiments, n is 2 or 3, each R¹ is independently selected from F and C and R², R² is OCH₃. In some embodiments, n is 2 or 3, each R¹ is independently selected from F and C and R² is absent.

In some embodiments, the compound of Formula I is a compound of Formula I-A or a pharmaceutically acceptable salt, solvate and/or prodrug thereof:

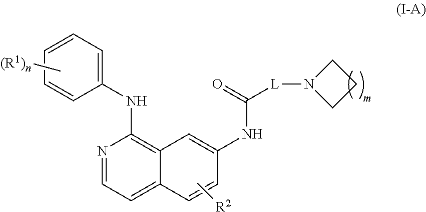

wherein
R¹, R², L and n are as defined in Formula I; and
m is an integer selected from 1 to 3.

In some embodiments, the compound of Formula I is selected from the compounds listed below:

| Compound | | |
|---|---|---|
| I.D | Chemical Name | Structure |
| 1-1 | N-(1-((3-chloro-4- fluorophenyl)amino)-6- methoxyisoquinolin-7-yl)-4- (piperidin-1-yl)butanamide | 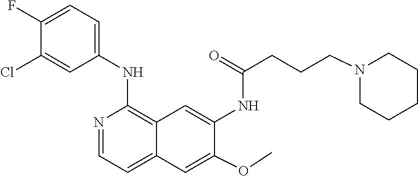 [SMILES: COc1cc2ccnc(Nc3ccc(F)c(Cl)c3)c2cc1NC(=O)CCCN1CCCCC1] |
| 1-2 | N-(1-((3-chloro-4- fluorophenyl)amino)-6- methoxyisoquinolin-7-yl)-5- (piperidin-1-yl)pentanamide | 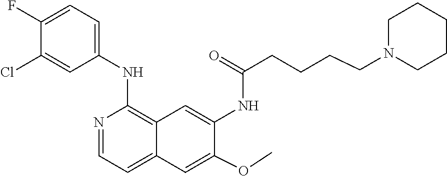 [SMILES: COc1cc2ccnc(Nc3ccc(F)c(Cl)c3)c2cc1NC(=O)CCCCN1CCCCC1] |
| 1-3 | N-(1-((3-chloro-4- fluorophenyl)amino)-6- methoxyisoquinolin-7-yl)-3- (piperidin-1-yl)propanamide | 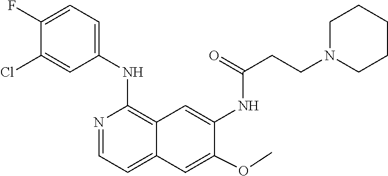 [SMILES: COc1cc2ccnc(Nc3ccc(F)c(Cl)c3)c2cc1NC(=O)CCN1CCCCC1] |
| 1-4 | N-(1-((3-chloro-4- fluorophenyl)amino)-6- methoxyisoquinolin-7-yl)-3- (piperidin-1-yl)propanamide | 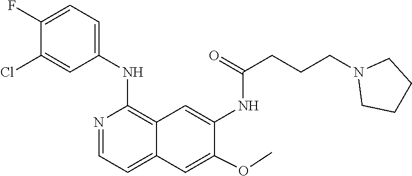 [SMILES: COc1cc2ccnc(Nc3ccc(F)c(Cl)c3)c2cc1NC(=O)CCCN1CCCC1] |
| 1-5 | N-(1-((3-chloro-4- fluorophenyl)amino)-6- methoxyisoquinolin-7-yl)-4- ((2S,6R)-2,6- dimethylmorpholino) butanamide | 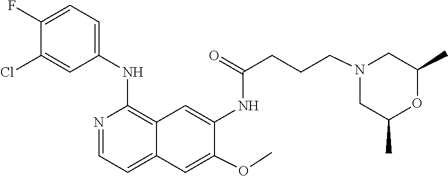 [SMILES: COc1cc2ccnc(Nc3ccc(F)c(Cl)c3)c2cc1NC(=O)CCCN1C[C@@H](C)O[C@@H](C)C1] |
| 1-6 | N-(1-((3-chloro-4- fluorophenyl)amino)-6- methoxyisoquinolin-7-yl)-4- morpholinobutanamide | 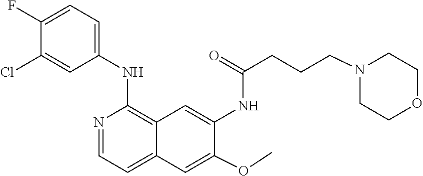 [SMILES: COc1cc2ccnc(Nc3ccc(F)c(Cl)c3)c2cc1NC(=O)CCCN1CCOCC1] |
| 1-7 | N-(1-((3-chloro-4- fluorophenyl)amino)-6- methoxyisoquinolin-7-yl)-4- (4-methylpiperazin-1- yl)butanamide | 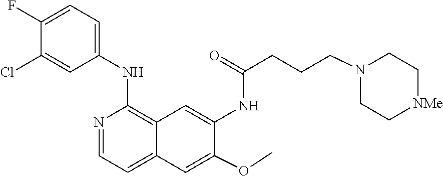 [SMILES: COc1cc2ccnc(Nc3ccc(F)c(Cl)c3)c2cc1NC(=O)CCCN1CCN(C)CC1] |
| 1-8 | N-(1-((3-chloro-4- fluorophenyl)amino)-6- methoxyisoquinolin-7-yl)-4- (4,4-difluoropiperidin-1- yl)butanamide | 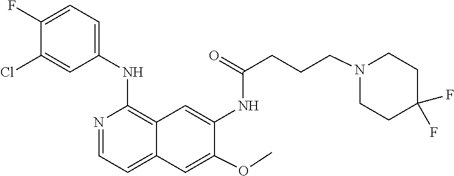 [SMILES: COc1cc2ccnc(Nc3ccc(F)c(Cl)c3)c2cc1NC(=O)CCCN1CCC(F)(F)CC1] |
| 1-9 | N-(1-((3- fluorophenyl)amino)-6- methoxyisoquinolin-7-yl)-4- (piperidin-1-yl)butanamide | 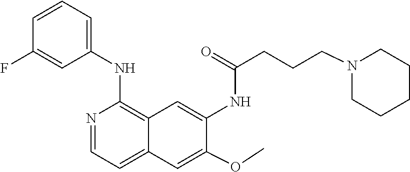 [SMILES: COc1cc2ccnc(Nc3cccc(F)c3)c2cc1NC(=O)CCCN1CCCCC1] |
| 1-10 | N-(6-methoxy-1-((3- methoxyphenyl)amino) isoquinolin-7-yl)-4-(piperidin- 1-yl)butanamide | 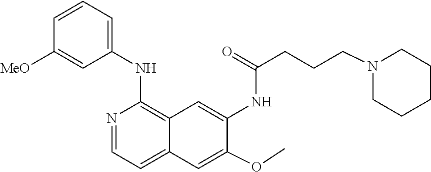 [SMILES: COc1cccc(Nc2nccc3cc(OC)c(NC(=O)CCCN4CCCCC4)cc23)c1] |
| 1-11 | N-(6-methoxy-1-(m- tolylamino)isoquinolin-7-yl)- 4-(piperidin-1-yl)butanamide | 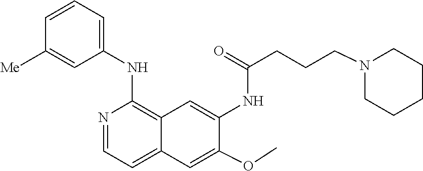 [SMILES: COc1cc2ccnc(Nc3cccc(C)c3)c2cc1NC(=O)CCCN1CCCCC1] |
| 1-12 | N-(1-((4- fluorophenyl)amino)-6- methoxyisoquinolin-7-yl)-4- (piperidin-1-yl)butanamide | 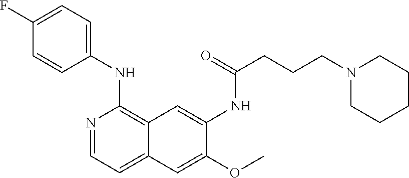 [SMILES: COc1cc2ccnc(Nc3ccc(F)cc3)c2cc1NC(=O)CCCN1CCCCC1] |
| 1-13 | N-(1-((3- chlorophenyl)amino)-6- methoxyisoquinolin-7-yl)-4- (piperidin-1-yl)butanamide | 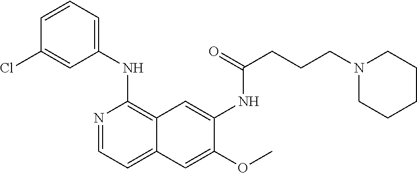 [SMILES: COc1cc2ccnc(Nc3cccc(Cl)c3)c2cc1NC(=O)CCCN1CCCCC1] |
| 1-14 | N-(6-methoxy-1-(p- tolylamino)isoquinolin-7-yl)- 4-(piperidin-1-yl)butanamide | 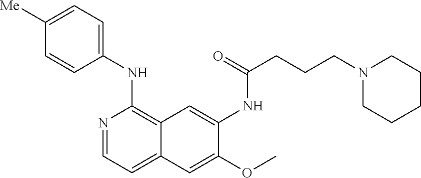 [SMILES: COc1cc2ccnc(Nc3ccc(C)cc3)c2cc1NC(=O)CCCN1CCCCC1] |
| 1-15 | N-(6-methoxy-1- (phenylamino)isoquinolin-7- yl)-4-(piperidin-1- yl)butanamide | 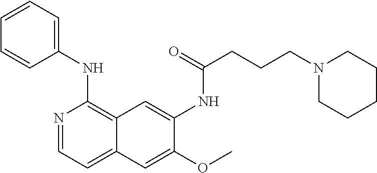 [SMILES: COc1cc2ccnc(Nc3ccccc3)c2cc1NC(=O)CCCN1CCCCC1] |
| 1-16 | N-(1-((4-fluoro-3- (trifluoromethyl)phenyl)amino)- 6-methoxyisoquinolin-7- yl)-4-(piperidin-1- yl)butanamide | 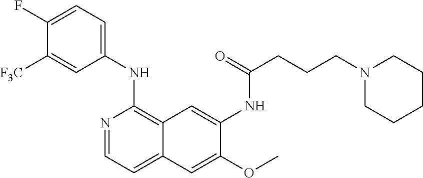 [SMILES: COc1cc2ccnc(Nc3ccc(F)c(C(F)(F)F)c3)c2cc1NC(=O)CCCN1CCCCC1] |
| 1-17 | N-(1-((3-chloro-4- fluorophenyl)amino)-6- methylisoquinolin-7-yl)-4- (piperidin-1-yl)butanamide | 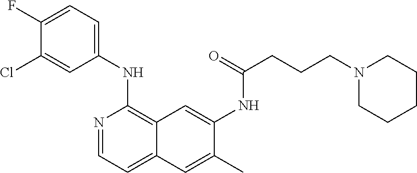 [SMILES: Cc1cc2ccnc(Nc3ccc(F)c(Cl)c3)c2cc1NC(=O)CCCN1CCCCC1] |
| 1-18 | N-(1-((3-chloro-4- fluorophenyl)amino)isoquinolin- 7-yl)-4-(piperidin-1- yl)butanamide | 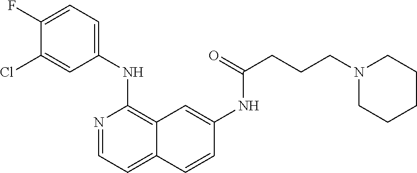 [SMILES: O=C(CCCN1CCCCC1)Nc1ccc2ccnc(Nc3ccc(F)c(Cl)c3)c2c1] |
| 1-19 | N-(1-((2-chloro-4- fluorophenyl)amino)-6- methoxyisoquinolin-7-yl)-4- (piperidin-1-yl)butanamide | 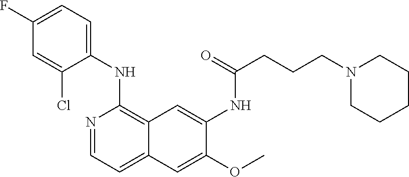 [SMILES: COc1cc2ccnc(Nc3ccc(F)cc3Cl)c2cc1NC(=O)CCCN1CCCCC1] |
| 1-20 | N-(1-((4-fluoro-3- methylphenyl)amino)-6- methoxyisoquinolin-7-yl)-4- (piperidin-1-yl)butanamide | 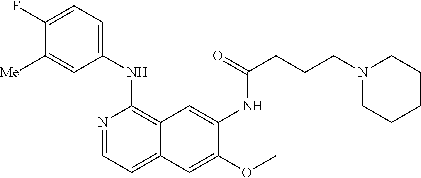 [SMILES: COc1cc2ccnc(Nc3ccc(F)c(C)c3)c2cc1NC(=O)CCCN1CCCCC1] |
| 1-21 | N-(1-((3-chloro-4- methylphenyl)amino)-6- methoxyisoquinolin-7-yl)-4- (piperidin-1-yl)butanamide | 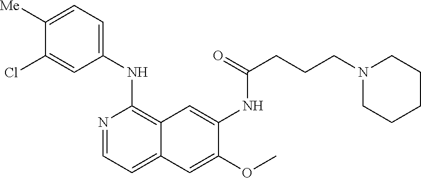 [SMILES: COc1cc2ccnc(Nc3ccc(C)c(Cl)c3)c2cc1NC(=O)CCCN1CCCCC1] |
| 1-22 | N-(1-((4-fluoro-3- methoxyphenyl)amino)-6- methoxyisoquinolin-7-yl)-4- (piperidin-1-yl)butanamide | 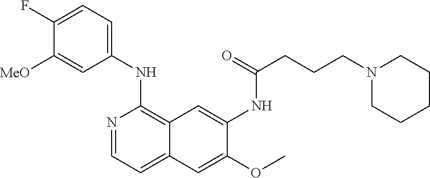 [SMILES: COc1cc(Nc2nccc3cc(OC)c(NC(=O)CCCN4CCCCC4)cc23)ccc1F] |
| 1-23 | N-(1-((3,5-dichloro-4- fluorophenyl)amino)-6- methoxyisoquinolin-7-yl)-4- (piperidin-1-yl)butanamide | 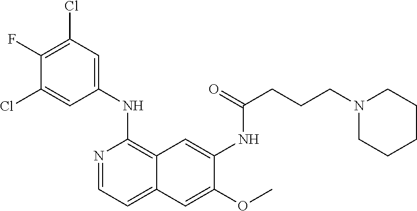 [SMILES: COc1cc2ccnc(Nc3cc(Cl)c(F)c(Cl)c3)c2cc1NC(=O)CCCN1CCCCC1] |
| 1-24 | N-(1-((3-chloro-4- methoxyphenyl)amino)-6- methoxyisoquinolin-7-yl)-4- (piperidin-1-yl)butanamide | 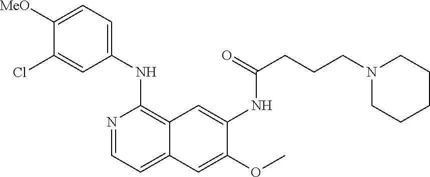 [SMILES: COc1ccc(Nc2nccc3cc(OC)c(NC(=O)CCCN4CCCCC4)cc23)cc1Cl] |
| 1-25 | N-(1-((3-chloro-5- fluorophenyl)amino)-6- methoxyisoquinolin-7-yl)-4- (piperidin-1-yl)butanamide | 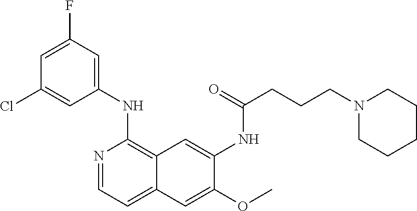 [SMILES: COc1cc2ccnc(Nc3cc(F)cc(Cl)c3)c2cc1NC(=O)CCCN1CCCCC1] |
| 1-26 | N-(6-chloro-1-((3-chloro-4- fluorophenyl)amino) isoquinolin-7-yl)-4-(piperidin- 1-yl)butanamide | 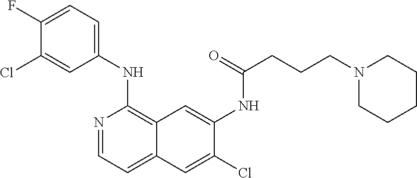 [SMILES: O=C(CCCN1CCCCC1)Nc1cc2c(Nc3ccc(F)c(Cl)c3)nccc2cc1Cl] |
| 1-27 | N-(1-((3,4- dichlorophenyl)amino)-6- methoxyisoquinolin-7-yl)-4- (piperidin-1-yl)butanamide | 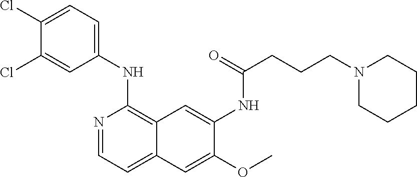 [SMILES: COc1cc2ccnc(Nc3ccc(Cl)c(Cl)c3)c2cc1NC(=O)CCCN1CCCCC1] |
| 1-28 | N-(1-((3-ethyl-4- fluorophenyl)amino)-6- methoxyisoquinolin-7-yl)-4- (piperidin-1-yl)butanamide | 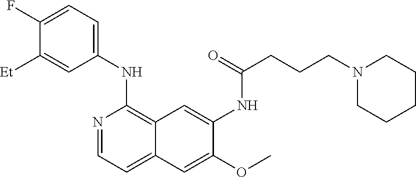 [SMILES: CCc1cc(Nc2nccc3cc(OC)c(NC(=O)CCCN4CCCCC4)cc23)ccc1F] |
| 1-29 | N-(1-((3-chloro-4- fluorophenyl)amino)-5- methoxyisoquinolin-7-yl)-4- (piperidin-1-yl)butanamide | 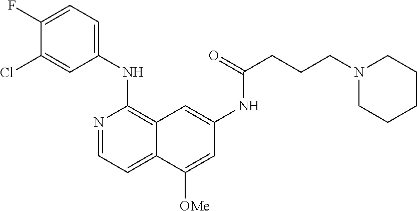 [SMILES: COc1cc(NC(=O)CCCN2CCCCC2)cc2c(Nc3ccc(F)c(Cl)c3)nccc12] |
| 1-30 | N-(1-((4-fluoro-3- isopropylphenyl)amino)-6- methoxyisoquinolin-7-yl)-4- (piperidin-1-yl)butanamide | 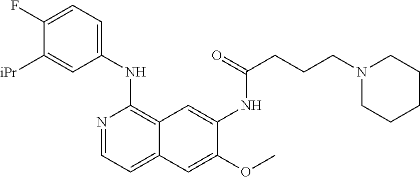 [SMILES: COc1cc2ccnc(Nc3ccc(F)c(C(C)C)c3)c2cc1NC(=O)CCCN1CCCCC1] |
| 1-31 | N-(1-((3-chloro-4- fluorophenyl)amino)-6- methoxyisoquinolin-7-yl)-N- methyl-4-(piperidin-1- yl)butanamide | 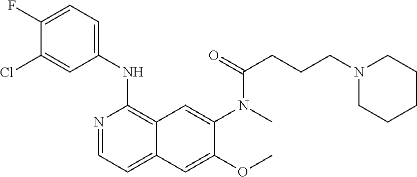 [SMILES: COc1cc2ccnc(Nc3ccc(F)c(Cl)c3)c2cc1N(C)C(=O)CCCN1CCCCC1] |
| 1-32 | N-(1-((3-chloro-4- methylphenyl)amino) isoquinolin-7-yl)-4-(piperidin- 1-yl)butanamide | 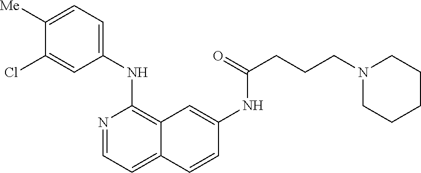 [SMILES: Cc1ccc(Nc2nccc3ccc(NC(=O)CCCN4CCCCC4)cc23)cc1Cl] |
| 1-33 | N-(1-((4-fluoro-3- (trifluoromethyl)phenyl) amino)isoquinolin-7-yl)-4- (piperidin-1-yl)butanamide | 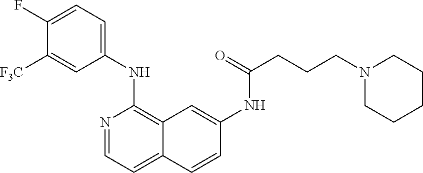 [SMILES: O=C(CCCN1CCCCC1)Nc1ccc2ccnc(Nc3ccc(F)c(C(F)(F)F)c3)c2c1] |
| 1-34 | N-(1-((3,4-dichloro-2- fluorophenyl)amino) isoquinolin-7-yl)-4-(piperidin- 1-yl)butanamide | 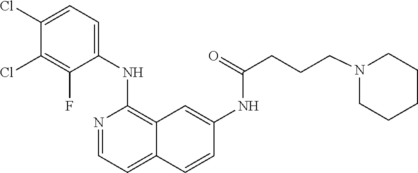 [SMILES: O=C(CCCN1CCCCC1)Nc1ccc2ccnc(Nc3ccc(Cl)c(Cl)c3F)c2c1] |
| 1-35 | N-(1-((3,4- dichlorophenyl)amino) isoquinolin-7-yl)-4-(piperidin- 1-yl)butanamide | 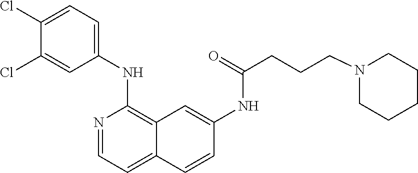 [SMILES: O=C(CCCN1CCCCC1)Nc1ccc2ccnc(Nc3ccc(Cl)c(Cl)c3)c2c1] |
| 1-36 | N-(1-((3,5-dichloro-4- fluorophenyl)amino)isoquinolin- 7-yl)-4-(piperidin-1- yl)butanamide | 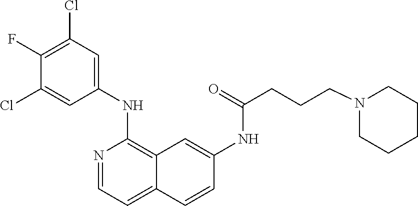 [SMILES: O=C(CCCN1CCCCC1)Nc1ccc2ccnc(Nc3cc(Cl)c(F)c(Cl)c3)c2c1] |
| 1-37 | N-(1-((3,4-dichloro-5- fluorophenyl)amino)isoquinolin- 7-yl)-4-(piperidin-1- yl)butanamide | 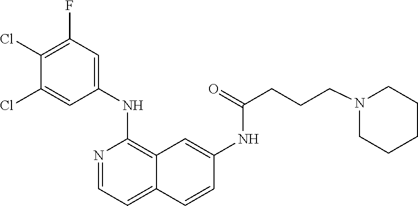 [SMILES: O=C(CCCN1CCCCC1)Nc1ccc2ccnc(Nc3cc(F)c(Cl)c(Cl)c3)c2c1] |
| 1-38 | N-(1-((4,5-dichloro-2- fluorophenyl)amino)isoquinolin- 7-yl)-4-(piperidin-1- yl)butanamide | 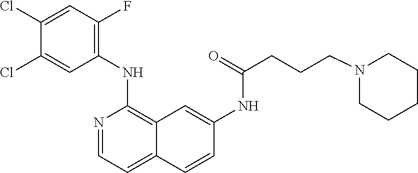 [SMILES: O=C(CCCN1CCCCC1)Nc1ccc2ccnc(Nc3cc(Cl)c(Cl)cc3F)c2c1] |
| 1-39 | N-(1-((3-chloro-2- fluorophenyl)amino)isoquinolin- 7-yl)-4-(piperidin-1- yl)butanamide | 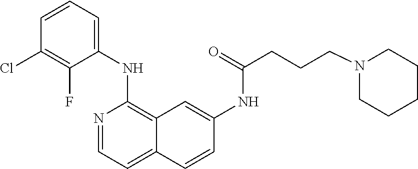 [SMILES: O=C(CCCN1CCCCC1)Nc1ccc2ccnc(Nc3cccc(Cl)c3F)c2c1] |
| 1-40 | N-(1-((3-chloro-2,4- difluorophenyl)amino) isoquinolin-7-yl)-4-(piperidin- 1-yl)butanamide | 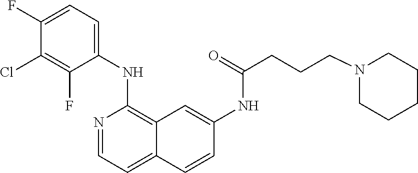 [SMILES: O=C(CCCN1CCCCC1)Nc1ccc2ccnc(Nc3ccc(F)c(Cl)c3F)c2c1] |
| 1-41 | N-(1-((3- cyanophenyl)amino) isoquinolin-7-yl)-4-(piperidin- 1-yl)butanamide | 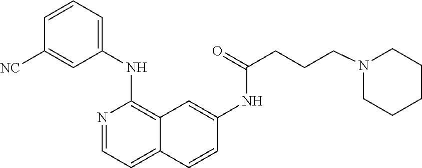 [SMILES: N#Cc1cccc(Nc2nccc3ccc(NC(=O)CCCN4CCCCC4)cc23)c1] |
| 1-42 | N-(1-((3-chloro-4- fluorophenyl)amino)-6- methoxyisoquinolin-7-yl)-3- (pyrrolidin-1-yl)propanamide | 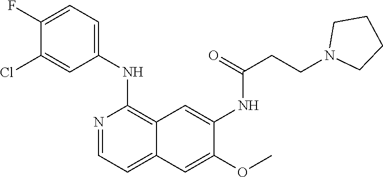 [SMILES: COc1cc2ccnc(Nc3ccc(F)c(Cl)c3)c2cc1NC(=O)CCN1CCCC1] |
| 1-43 | N-(1-((3-chloro-4- fluorophenyl)amino)-6- methoxyisoquinolin-7-yl)-4- (3,3-difluoropyrrolidin-1- yl)butanamide | 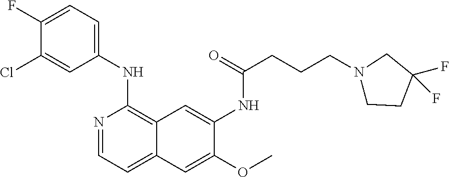 [SMILES: COc1cc2ccnc(Nc3ccc(F)c(Cl)c3)c2cc1NC(=O)CCCN1CCC(F)(F)C1] |
| 1-44 | N-(1-((3,5-dichloro-2- fluorophenyl)amino) isoquinolin-7-yl)-4-(piperidin- 1-yl)butanamide | 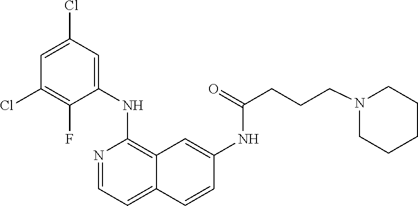 [SMILES: O=C(CCCN1CCCCC1)Nc1ccc2ccnc(Nc3cc(Cl)cc(Cl)c3F)c2c1] |
| 1-45 | N-(1-((4-chloro-2,3- difluorophenyl)amino) isoquinolin-7-yl)-4-(piperidin- 1-yl)butanamide | 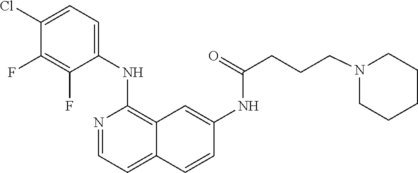 [SMILES: O=C(CCCN1CCCCC1)Nc1ccc2ccnc(Nc3ccc(Cl)c(F)c3F)c2c1] |
| 1-46 | N-(1-((3-chloro-4- fluorophenyl)amino)-6- ethoxyisoquinolin-7-yl)-4- (piperidin-1-yl)butanamide | 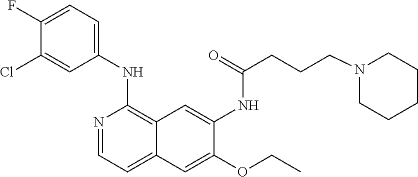 [SMILES: CCOc1cc2ccnc(Nc3ccc(F)c(Cl)c3)c2cc1NC(=O)CCCN1CCCCC1] |
| (R) I-47 | (R)-N-(1-((3-chloro-4- fluorophenyl)amino)-6- methoxyisoquinolin-7-yl)-4- (3-methoxypyrrolidin-1- yl)butanamide | 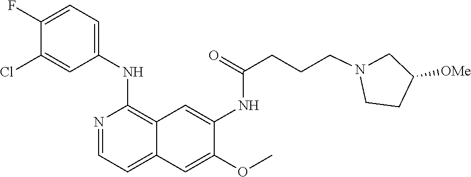 [SMILES: COc1cc2ccnc(Nc3ccc(F)c(Cl)c3)c2cc1NC(=O)CCCN1CC[C@@H](OC)C1] |
| (S) 1-47 | (S)-N-(1-((3-chloro-4- fluorophenyl)amino)-6- methoxyisoquinolin-7-yl)-4- (3-methoxypyrrolidin-1- yl)butanamide | 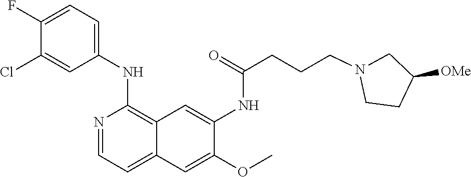 [SMILES: COc1cc2ccnc(Nc3ccc(F)c(Cl)c3)c2cc1NC(=O)CCCN1CC[C@H](OC)C1] |
| (R) 1-48 | (R)-N-(1-((3-chloro-4- fluorophenyl)amino)-6- methoxyisoquinolin-7-yl)-4- (3-fluoropyrrolidin-1- yl)butanamide | 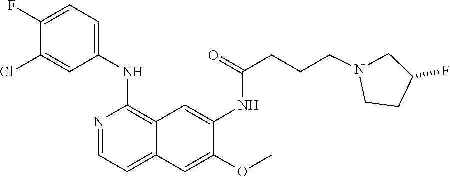 [SMILES: COc1cc2ccnc(Nc3ccc(F)c(Cl)c3)c2cc1NC(=O)CCCN1CC[C@@H](F)C1] |
| (S) I-48 | (S)-N-(1-((3-chloro-4- fluorophenyl)amino)-6- methoxyisoquinolin-7-yl)-4- (3-fluoropyrrolidin-1- yl)butanamide | 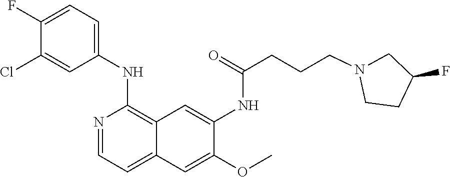 [SMILES: COc1cc2ccnc(Nc3ccc(F)c(Cl)c3)c2cc1NC(=O)CCCN1CC[C@H](F)C1] |
| 1-49 | N-(1-((3-chloro-4- fluorophenyl)amino)-6- propoxyisoquinolin-7-yl)-4- (piperidin-1-yl)butanamide | 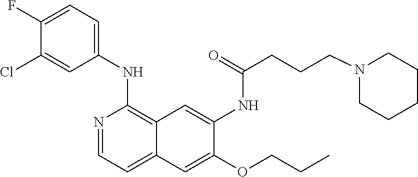 [SMILES: CCCOc1cc2ccnc(Nc3ccc(F)c(Cl)c3)c2cc1NC(=O)CCCN1CCCCC1] |
| 1-50 | N-(1-((3-chloro-4- fluorophenyl)amino)-6- fluoroisoquinolin-7-yl)-4- (piperidin-1-yl)butanamide | 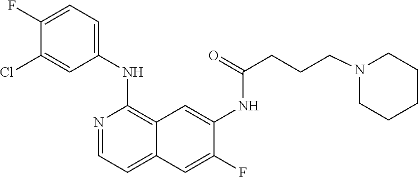 [SMILES: O=C(CCCN1CCCCC1)Nc1cc2c(Nc3ccc(F)c(Cl)c3)nccc2cc1F] |
| 1-51 | N-(1-((3-chloro-4- fluorophenyl)amino)-5- fluoroisoquinolin-7-yl)-4- (piperidin-1-yl)butanamide | 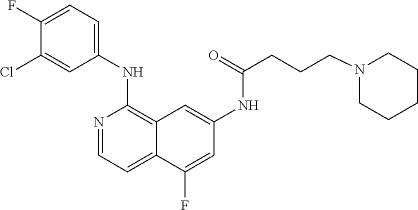 [SMILES: O=C(CCCN1CCCCC1)Nc1cc(F)c2ccnc(Nc3ccc(F)c(Cl)c3)c2c1] |
| 1-52 | (E)-N-(1-((3-chloro-4- fluorophenyl)amino) isoquinolin-7-yl)-4-(piperidin- 1-yl)but-2-enamide | 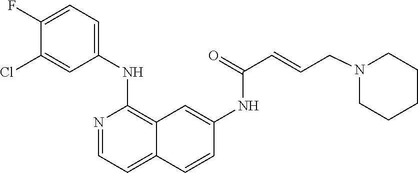 [SMILES: O=C(/C=C/CN1CCCCC1)Nc1ccc2ccnc(Nc3ccc(F)c(Cl)c3)c2c1] |
| 1-53 | N-(1-((3-chloro-4- fluorophenyl)amino)-4- fluoroisoquinolin-7-yl)-4- (piperidin-1-yl)butanamide | 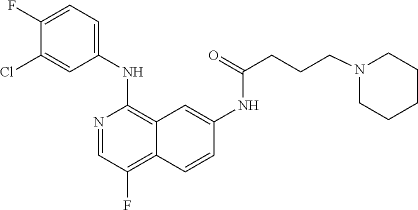 [SMILES: O=C(CCCN1CCCCC1)Nc1ccc2c(F)cnc(Nc3ccc(F)c(Cl)c3)c2c1] |

or a pharmaceutically acceptable salt, solvate and/or prodrug thereof:

In some embodiments, the compound of Formula I is

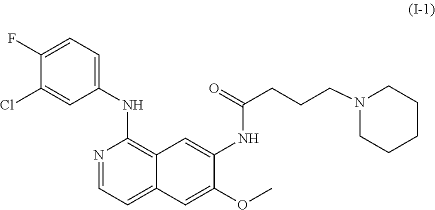 [SMILES: COc1cc2ccnc(Nc3ccc(F)c(Cl)c3)c2cc1NC(=O)CCCN1CCCCC1]

or a pharmaceutically acceptable salt, solvate and/or prodrug thereof.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the application include, for example, those made with solvents that are pharmaceutically acceptable. Examples of such solvents include water (resulting solvate is called a hydrate) and ethanol and the like. Suitable solvents are physiologically tolerable at the dosage administered.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

The compounds of the present application may further be radiolabeled and accordingly all radiolabeled versions of the compounds of the application are included within the scope of the present application. The compounds of the application also include those in which one or more radioactive atoms are incorporated within their structure.

### III. Compositions of the Application

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In embodiments of the application the pharmaceutical compositions are used in the treatment of any of the diseases, disorders or conditions described herein.

The compounds of the application are administered to a subject in a variety of forms depending on the selected route of administration, as will be understood by those skilled in the art. For example, a compound of the application is administered by oral, inhalation, parenteral, buccal, sublingual, nasal, rectal, vaginal, patch, pump, minipump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In some embodiments, compounds of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers

A compound of the application including pharmaceutically acceptable salts and/or solvates thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### IV. Methods and Uses of the Application

The compounds of the application have been shown to inhibit or block Bcl2-associated X protein (BAX) and/or Bcl-2 antagonist killer (BAK). Therefore, the compounds of the application are useful for inhibiting BAX and/or BAK.

Accordingly, the present application includes a method of inhibiting Bcl2-associated X protein (BAX) and/or Bcl-2 antagonist killer (BAK) in a cell, either in a biological sample or in a patient, comprising administering an effective amount of one or more compounds of the application to the cell.

The present application also includes a use of one or more compounds of the application for inhibiting BAX and/or BAK in a cell as well as a use of one or more compounds of the application for the preparation of a medicament for inhibiting BAX and/or BAK in a cell. The application further includes one or more compounds of the application for use in inhibiting BAX and/or BAK in a cell.

In some embodiments, the methods and uses are for inhibiting Bcl2-associated X protein (BAX) in a cell, either in a biological sample or in a patient. In some embodiments, the methods and uses are for inhibiting Bcl-2 antagonist killer (BAK) in a cell, either in a biological sample or in a patient. In some embodiments, the methods and uses are for inhibiting both Bcl2-associated X protein (BAX) and Bcl-2 antagonist killer (BAK) in a cell, either in a biological sample or in a patient.

In some embodiments, inhibiting Bcl2-associated X protein (BAX) and/or Bcl-2 antagonist killer (BAK) inhibits or prevents Bcl2-associated X protein (BAX) and/or Bcl-2 antagonist killer (BAK) mediated cell death. Therefore, in some embodiments, the methods and uses are for treating or preventing BAX and/or BAK mediated cell death in a cell either in a biological sample or in a patient. In some embodiments, the BAX and/or BAK mediated cell death is apoptosis.

In some embodiments, inhibiting BAX and/or BAK inhibits or prevents mitochondrial outer membrane permeabilization (MOMP). Therefore, in some embodiments, the methods and uses are for inhibiting MOMP in a cell, either in a biological sample or in a patient.

In some embodiments, inhibiting BAX and/or BAK inhibits BAX and BAK oligomerization. Therefore, in some embodiments, the methods and uses are for inhibiting BAX and BAK oligomerization in a cell, either in a biological sample or in a patient.

As the compounds of the application have been shown to be capable of inhibiting BAX and/or BAK protein activity, the compounds of the application are useful for treating diseases, disorders or conditions by inhibiting BAX and/or BAK. Therefore, the compounds of the present application are useful as medicaments. Accordingly, the present application includes a compound of the application for use as a medicament.

Accordingly, the present application also includes a method of treating a disease, disorder or condition that is treatable by inhibiting Bcl2-associated X protein (BAX) and/or Bcl-2 antagonist killer (BAK) comprising administering a therapeutically effective amount of one or more compounds of the application to a subject in need thereof. In some embodiments, the subject is a subject having said disease, disorder or condition.

The present application also includes a use of one or more compounds of the application for treating a disease, disorder or condition that is treatable by inhibiting BAX and/or BAK, as well as a use of one or more compounds of the application for the preparation of a medicament for treating a disease, disorder or condition that is treatable by inhibiting BAX and/or BAK. The application further includes one or more compounds of the application for use in treating a disease, disorder or condition that is treatable by inhibiting BAX and/or BAK.

In some embodiments, the disease, disorder or condition that is treatable by inhibiting BAX and/or BAK is a neurodegenerative disease, disorder or condition. Therefore, in some embodiments, the methods and uses are for treating neurodegenerative diseases, disorders or conditions. In some embodiments, the neurodegenerative disease, disorder or condition. is selected from Alzheimer's Disease, Huntington's Disease, Parkinson's Disease, Friedreich's ataxia, amyotrophic lateral sclerosis (ALS), multiple sclerosis, ischemic brain injury, glaucoma, encephalitis, meningitis, and trauma-induced inflammatory neuronal damage including, for example, malaria encephalitis or cerebral malaria.

In some embodiments, the disease, disorder or condition that is treatable by inhibiting BAX and/or BAK is neuronal damage associated with ischemia. Therefore, in some embodiments, the methods and uses are for treating neuronal damage associated with ischemia. In some embodiments, the neuronal damage associated with ischemia is from a stroke. Therefore, the methods and uses are for treating neuronal damage associated with a stroke.

In some embodiments, the disease, disorder or condition that is treatable by inhibiting BAX and/or BAK is cardiomyopathy induced by a chemotherapeutic agent. Therefore, in some embodiments, the methods and uses are for treating cardiomyopathy induced by a chemotherapeutic agent. In some embodiments, the chemotherapeutic agent is doxorubicin. Therefore, in some embodiments, the methods and uses are for treating cardiomyopathy induced by doxorubicin.

In some embodiments, the disease, disorder or condition that is treatable by inhibiting BAX and/or BAK is donor hematopoietic stem and progenitor cells (HSPCs) cell death. Therefore, in some embodiments, the methods and uses are for treating donor hematopoietic stem and progenitor cells (HSPCs) cell death. In some embodiments, the treating donor hematopoietic stem and progenitor cells (HSPCs), cell death is during transplantation and includes all stages of the transplant process including, for example, removal of HSPCs from original niche, transportation, cryopreservation and engraftment. In some embodiments, the treating donor HSPCs cell death is by inhibiting or reducing donor HSPCs cell death.

Accordingly, the present application includes a method for increasing the survival of donor HSPCs during transplantation comprising administering a therapeutically effective amount of one or more compounds of the application to a subject in need thereof.

The present application also includes the use of one or more compounds of the application for increasing the survival of donor HSPCs during transplantation, as well as a use of one or more compounds of the application for the preparation of a medicament for increasing the survival of donor HSPCs during transplantation. The application further includes one or more compounds of the application for use in increasing the survival of donor HSPCs during transplantation.

In some embodiments, the methods and uses are for treating or preventing BAX and/or BAK mediated cell death in cells and thereby increase the survival of any cells, for example cells used in a cell-based therapy. Accordingly, the present application includes a method of increasing the survival of cells comprising administering an effective amount of one or more compounds of the application to cells in need thereof. Also included is a use of one or more compounds of the application for increasing the survival of cells, a use of one or more compounds of the application for preparation of a composition for increasing the survival of cells and one or more compounds of the application for use to increase the survival of cells. In some embodiments, the cells are any cells used in cell-based therapy. In some embodiments the cells used in cell-based therapy are, for example but not limited to, transplanted skin cells in a burn patient, hair follicles for baldness, corneal endothelial cells in Fuch's corneal dystrophy and the like.

In some embodiments, the treating the disease, disorder or condition that is treatable by inhibiting BAX and/or BAK is by preventing the disease, disorder or condition that is treatable by inhibiting BAX and/or BAK. Therefore, in some embodiments, the methods and uses are for preventing neurodegenerative diseases, disorders or conditions, preventing neuronal damage associated with ischemia, preventing cardiomyopathy induced by a chemotherapeutic agent and/or preventing donor HSPCs cell death.

The present application also includes a method of treating a disease, disorder or condition that is treatable by inhibiting BAX and/or BAK comprising administering a therapeutically effective amount of one or more compounds of the application in combination with another known agent useful for treatment of a disease, disorder or condition that is treatable by inhibiting BAX and/or BAK to a subject in need thereof. In some embodiments, the subject is a subject having said disease, disorder or condition.

The present application also includes a use of one or more compounds of the application in combination with another known agent useful for treatment of a disease, disorder or condition that is treatable by inhibiting BAX and/or BAK, as well as a use of one or more compounds of the application in combination with another known agent useful for treatment of a disease, disorder or condition that is treatable by inhibiting BAX and/or BAK for the preparation of a medicament for treatment of a disease, disorder or condition that is treatable by inhibiting BAX and/or BAK. The application further includes one or more compounds of the application in combination with another known agent useful for treatment of a disease, disorder or condition that is treatable by inhibiting BAX and/or BAK.

In an embodiment, the disease, disorder or condition that is treatable by inhibiting BAX and/or BAK is a neurodegenerative disease, disorder or condition, neuronal damage associated with ischemia, cardiomyopathy induced by a chemotherapeutic agent and/or donor hematopoietic stem and progenitor cells (HSPCs) cell death.

In an embodiment, the subject is a mammal. In another embodiment, the subject is human.

Compounds of the application are either used alone or in combination with other known agents useful for treating a disease, disorder or condition that is treatable by inhibiting BAX and/or BAK. When used in combination with other agents useful in treating diseases, disorders or conditions that are treatable by inhibiting BAX and/or BAK, it is an embodiment that the compounds of the application are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other and can include administering the two substances within a few hours of each other, or even administering one substance within 24 hours of administration of the other if the pharmacokinetics are suitable. Design of suitable dosing regimens is routine for one skilled in the art. In particular, embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, compounds of the present application are administered with another therapeutic agent simultaneously or sequentially in separate unit dosage forms or together in a single unit dosage form. Accordingly, the present application provides a single unit dosage form comprising one or more compounds of the application (e.g. a compound of Formula I), an additional therapeutic agent, and a pharmaceutically acceptable carrier.

Treatment methods comprise administering to a subject a therapeutically effective amount of one or more of the compounds of the application and optionally consist of a single administration, or alternatively comprise a series of administrations, and optionally comprise concurrent administration or use of one or more other therapeutic agents. For example, in some embodiments, the compounds of the application may be administered at least once a week. In some embodiments, the compounds may be administered to the subject from about one time per two or three weeks, or about one time per week to about once daily for a given treatment. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration may be required. For example, the compounds are administered to the subject in an amount and for duration sufficient to treat the subject. In some embodiments treatment comprise prophylactic treatment. For example, a subject with early cancer can be treated to prevent progression, or alternatively a subject in remission can be treated with a compound or composition of the application to prevent recurrence.

The dosage of compounds of the application varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 0.05 mg per day to about 1000 mg per day for an adult, suitably about 0.1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. Compounds of the application may be administered in a single daily, weekly or monthly dose or the total daily dose may be divided into two, three or four daily doses.

In an embodiment, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

To be clear, in the above, the term “a compound” also includes embodiments wherein one or more compounds are referenced. Likewise, the term “compounds of the application” also includes embodiments wherein only one compound is referenced.

### V. Methods of Preparation of Compounds of the Application

Compounds of the present application can be prepared by various synthetic processes. The choice of particular structural features and/or substituents may influence the selection of one process over another. The selection of a particular process to prepare a given compound of Formula I is within the purview of the person of skill in the art. Some starting materials for preparing compounds of the present application are available from commercial chemical sources. Other starting materials, for example as described below, are readily prepared from available precursors using straightforward transformations that are well known in the art.

The compounds of Formula I generally can be prepared according to the processes illustrated in the Schemes below. In the structural formulae shown below the variables are as defined in Formula I unless otherwise stated. A person skilled in the art would appreciate that many of the reactions depicted in the Schemes below would be sensitive to oxygen and water and would know to perform the reaction under an anhydrous, inert atmosphere if needed. Reaction temperatures and times are presented for illustrative purposes only and may be varied to optimize yield as would be understood by a person skilled in the art.

Accordingly, in some embodiments, the compounds of Formula I, are prepared as shown in Scheme 1.

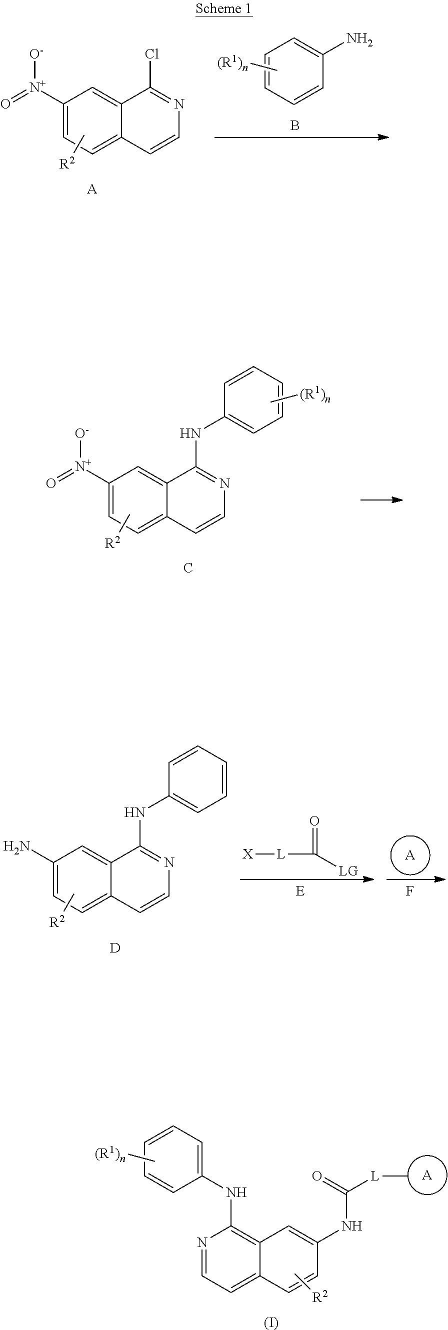

Therefore, in some embodiments, the compound of Formula A is coupled with an amino compound of Formula B to provide an intermediate compound of Formula C. The compound of Formula C is reduced under suitable conditions to provide the amino compound of Formula D. The compound of Formula D is then coupled with a compound of Formula E wherein X is halo such as Br and LG is a leaving groups such as Cl under suitable coupling conditions such as in the presence of an organic base such as N,N-diisopropylethylamine, and further coupled with a C₃₋₈heterocyclic compound including at least one ring heteromoiety selected from N and NH of Formula F to provide compounds of Formula I.

In embodiments, the compounds of Formula A are prepared as shown in Scheme 2.

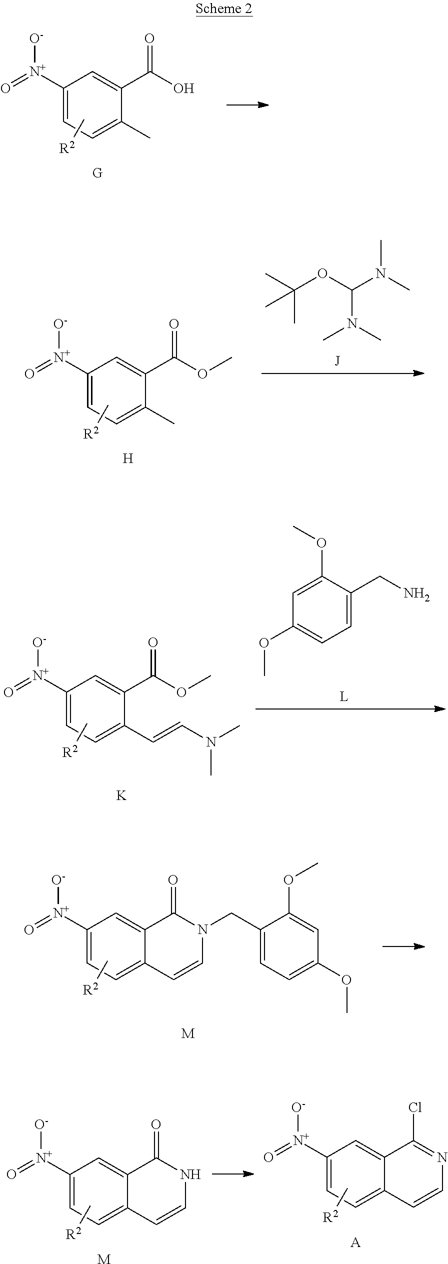

Therefore, in some embodiments, the compound of Formula G is esterified to form an ester, for example, methyl ester compound of Formula H which is then coupled with tert-butoxy bis(dimethylamino)methane compound of formula J under suitable conditions such as at temperature of about at 105° C. to 120° C. to provide the compound of Formula K. The compound of formula K is then cyclized in the presence of the 2,4-dimethoxybenzylamine compound of Formula L under suitable conditions such as such as at temperature of about at 105° C. to 120° C. in a suitable solvent such as toluene to provide the compound of Formula M. The 2,4-dimethoxybenzyl group is then removed from the compound of Formula M in the presence of an acid such as trifluoroacetic acid to provide the compound of Formula N which reacted with a chlorinating agent such as phosphorous (V) oxychloride to provide the compound of Formula A.

Generally, the reactions described above are performed in a suitable inert organic solvent and at temperatures and for times that will optimize the yield of the desired compounds. Examples of suitable inert organic solvents include, but are not limited to, 2-propanol, dimethylformamide (DMF), 1,4-dioxane, methylene chloride, chloroform, tetrahydrofuran (THF), toluene, and the like.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine).

The formation of a desired compound salt is achieved using standard techniques. For example, the neutral compound is treated with an acid or base in a suitable solvent and the formed salt is isolated by filtration, extraction or any other suitable method.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### EXAMPLES

The following non-limiting examples are illustrative of the present application:

### A. Synthesis and Characterization of Exemplary Compounds of the Application

### Example 1: Preparation of N-(1-((3-chloro-4-fluorophenyl)amino)-6-methoxyisoquinolin-7-yl)-4-(piperidin-1-yl)butanamide (I-1)

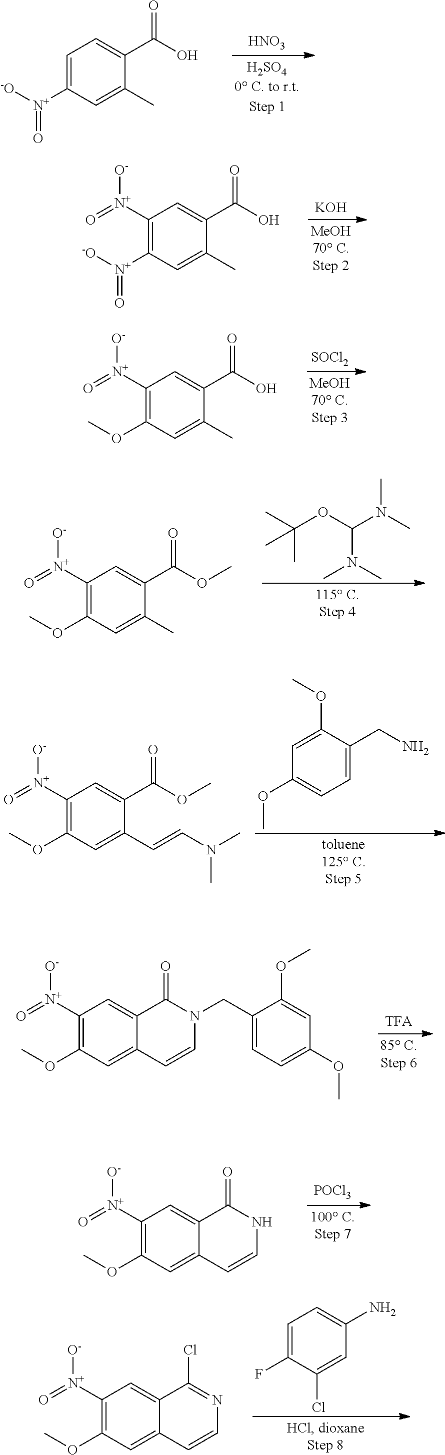

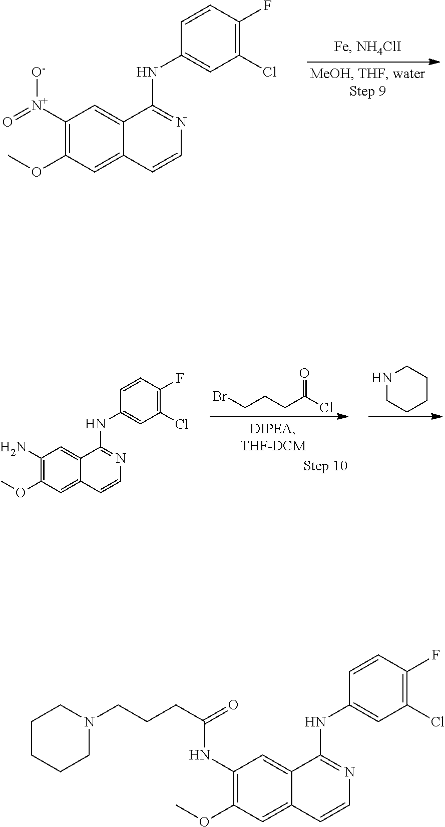 [SMILES: COc1cc2ccnc(Nc3ccc(F)c(Cl)c3)c2cc1[N+](=O)[O-] ; COc1cc2ccnc(Nc3ccc(F)c(Cl)c3)c2cc1N ; O=C(Cl)CCCBr ; C1CCNCC1 ; COc1cc2ccnc(Nc3ccc(F)c(Cl)c3)c2cc1NC(=O)CCCN1CCCCC1]

### Step 1: 2-Methyl-4,5-dinitrobenzoic acid

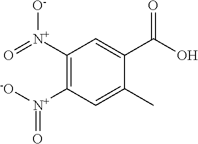

2-Methyl-4-nitrobenzoic acid (9 g, 49.7 mmol) was added in portions to a pre-mixed solution of nitric acid (31.3 g, 497 mmol) and sulfuric acid (97 g, 994 mmol) under ice-water bath. The reaction mixture was stirred at r.t. overnight, then poured in 300 g of ice, resulting solid was filtered, rinsed with water and hexanes, dried under vacuum to give crude 2-methyl-4,5-dinitrobenzoic acid (9.361 g) as off-white solid. Yield=83%, purity=72%. LCMS: [M−H]⁻=225.27. ¹H NMR (500 MHz, METHANOL-d4) δ=8.55 (s, 1H), 7.98 (s, 1H), 2.76 (s, 3H).

### Step 2: 4-Methoxy-2-methyl-5-nitrobenzoic acid

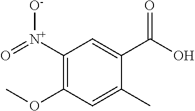

2-Methyl-4,5-dinitrobenzoic acid (9.361 g, 41.4 mmol) was mixed with potassium hydroxide (11.61 g, 207 mmol) in MeOH (120 mL) and the reaction mixture was stirred under 70° C. oil bath for 90 min. The reaction mixture was acidified with 2N HCl to pH<1, the resulting orange-red solid was filtered, washed with water, air dried overnight to give crude 4-methoxy-2-methyl-5-nitrobenzoic acid (8.5 g). Yield=97%, purity=76%. [M−H]⁻=210.16. ¹H NMR (500 MHz, DMSO-d₆) δ=13.45-12.99 (m, 1H), 8.43-8.29 (m, 1H), 7.32 (s, 1H), 3.99 (s, 3H), 2.64 (s, 3H).

### Step 3: Methyl 4-methoxy-2-methyl-5-nitrobenzoate

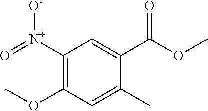 [SMILES: COC(=O)c1cc([N+](=O)[O-])c(OC)cc1C]

4-Methoxy-2-methyl-5-nitrobenzoic acid (8.5 g, 40.3 mmol) was mixed with MeOH (150 mL) under ice-water bath, then thionyl chloride (8.38 g, 70.4 mmol) was added dropwise. The reaction mixture was stirred under 70° C. oil bath for 4.5 hours, then at room temperature overnight. The reaction mixture was concentrated by rotary evaporator and the residue was dissolved in EtOAc and DCM, concentrated with silica gel, purified by Biotage (50 g) column, eluted with 0-15% EtOAc in hexanes. The product was triturated with ether to give methyl 4-methoxy-2-methyl-5-nitrobenzoate (2.56 g) as light yellow solid. Yield=28.2%. [M+H]⁺=226.34. ¹H NMR (500 MHz, DMSO-d₆) δ=8.38 (s, 1H), 7.36 (s, 1H), 4.00 (s, 3H), 3.83 (s, 3H), 2.63 (s, 3H)

### Step 4: Methyl (E)-2-(2-(dimethylamino)vinyl)-4-methoxy-5-nitrobenzoate

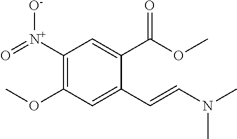 [SMILES: COC(=O)c1cc([N+](=O)[O-])c(OC)cc1/C=C/N(C)C]

Methyl 4-methoxy-2-methyl-5-nitrobenzoate (2.56 g, 11.37 mmol) was mixed with tert-Butoxy bis(dimethylamino)methane (2.58 g, 14.78 mmol) and heated at 115° C. oil bath for an hour and 20 minutes. The reaction mixture was diluted with hexanes and resulting solid was filtered, rinsed with hexanes to give methyl (E)-2-(2-(dimethylamino)vinyl)-4-methoxy-5-nitrobenzoate (2.946 g) as dark red solid. Yield=92%. ¹H NMR (500 MHz, DMSO-d₆) δ=8.40 (s, 1H), 7.73 (d, J=13.3 Hz, 1H), 7.17 (s, 1H), 6.28 (d, J=13.3 Hz, 1H), 3.98 (s, 3H), 3.79 (s, 3H), 2.98 (s, 6H).

### Step 5: 2-(2,4-Dimethoxybenzyl)-6-methoxy-7-nitroisoquinolin-1(2H)-one

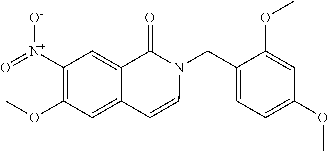 [SMILES: COc1ccc(Cn2ccc3cc(OC)c([N+](=O)[O-])cc3c2=O)c(OC)c1]

Methyl (E)-2-(2-(dimethylamino)vinyl)-4-methoxy-5-nitrobenzoate (2.96 g, 10.56 mmol) was mixed with 2,4-dimethoxybenzylamine (2.472 g, 14.79 mmol) in toluene (20 mL) and the reaction mixture was heated under 125° C. oil bath for an hour. The reaction mixture was cooled down and diluted with hexanes, filtered. The solid was triturated with to give 2-(2,4-dimethoxybenzyl)-6-methoxy-7-nitroisoquinolin-1(2H)-one (3.31 g) as yellow solid. Yield=85%. [M+H]⁺=371.35. ¹H NMR (500 MHz, DMSO-d₆) δ=8.60 (s, 1H), 7.57 (d, J=7.5 Hz, 1H), 7.49 (s, 1H), 7.04 (d, J=8.3 Hz, 1H), 6.64 (d, J=7.3 Hz, 1H), 6.59 (d, J=2.3 Hz, 1H), 6.47 (dd, J=2.3, 8.4 Hz, 1H), 5.01 (s, 2H), 4.01 (s, 3H), 3.81 (s, 3H), 3.74 (s, 3H).

### Step 6: 6-Methoxy-7-nitroisoquinolin-1(2H)-one

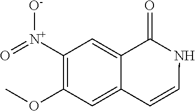 [SMILES: COc1cc2cc[nH]c(=O)c2cc1[N+](=O)[O-]]

2-(2,4-Dimethoxybenzyl)-6-methoxy-7-nitroisoquinolin-1(2H)-one (3.31 g, 8.94 mmol) was mixed with trifluoroacetic acid (50 mL) and the reaction mixture was stirred under 85° C. oil bath for three hours. The reaction mixture was concentrated using a rotary evaporator and the residue was mixed with MeOH and concentrated again, then triturated with EtOAc to give 6-methoxy-7-nitroisoquinolin-1(2H)-one (1.784 g) as pale-green solid. Yield=90.9%. [M+H]⁺=221.20. ¹H NMR (500 MHz, DMSO-d₆) δ=11.47 (br s, 1H), 8.57 (s, 1H), 7.51 (s, 1H), 7.40-7.30 (m, 1H), 6.58 (d, J=7.1 Hz, 1H), 4.01 (s, 3H)

### Step 7: 1-Chloro-6-methoxy-7-nitroisoquinoline

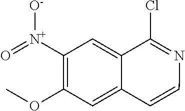 [SMILES: COc1cc2ccnc(Cl)c2cc1[N+](=O)[O-]]

6-Methoxy-7-nitroisoquinolin-1(2H)-one (1.784 g, 8.10 mmol) was mixed with phosphorous (V) oxychloride (37.3 g, 243 mmol) and the reaction mixture was stirred under 100° C. oil bath for an hour and 20 minutes, then concentrated by rotavapor to remove excess of POCl₃. The residue was diluted with DCM and ice-water, neutralized with solid NaHCO₃. DCM layer was concentrated with silica gel and purified using a Biotage column (10 g), eluting with 30-50% EtOAc in hexanes. The resulting product was triturated with ether to give 1-chloro-6-methoxy-7-nitroisoquinoline (1.016 g) as pale-yellow solid. Yield=53%. [M+H]⁺=239.26. ¹H NMR (500 MHz, DMSO-d₆) δ=8.75 (s, 1H), 8.38 (d, J=5.7 Hz, 1H), 7.91-7.86 (m, 2H), 4.07 (s, 3H)

### Step 8: N-(3-Chloro-4-fluorophenyl)-6-methoxy-7-nitroisoquinolin-1-amine hydrochloride

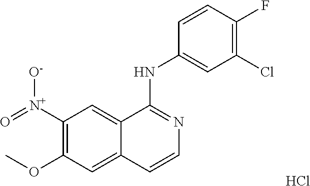 [SMILES: COc1cc2ccnc(Nc3ccc(F)c(Cl)c3)c2cc1[N+](=O)[O-] ; Cl]

1-Chloro-6-methoxy-7-nitroisoquinoline (400 mg, 1.676 mmol) was mixed with 3-chloro-4-fluoroaniline (244 mg, 1.68 mmol) in 1,4-dioxane (5 mL), then hydrochloric acid, 4.0 M in dioxane (0.84 mL, 3.35 mmol) was added. The reaction suspension was stirred under 100° C. oil bath for 2 hours, then diluted with ether, filtered. The solid was rinsed with ether to give N-(3-chloro-4-fluorophenyl)-6-methoxy-7-nitroisoquinolin-1-amine hydrochloride (676 mg). Yield=quantitative. [M−HCl+H]⁺=348.34. ¹H NMR (500 MHz, DMSO-d₆) 5=9.36 (s, 1H), 8.04 (br d, J=5.0 Hz, 1H), 7.90 (br d, J=5.7 Hz, 1H), 7.77 (s, 1H), 7.73-7.66 (m, 1H), 7.54 (br t, J=9.0 Hz, 1H), 7.30 (d, J=6.4 Hz, 1H), 4.08 (s, 3H)

### Step 9: N¹-(3-chloro-4-fluorophenyl)-6-methoxyisoquinoline-1,7-diamine

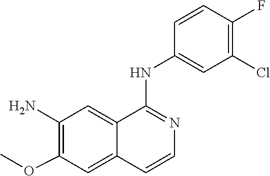 [SMILES: COc1cc2ccnc(Nc3ccc(F)c(Cl)c3)c2cc1N]

N-(3-chloro-4-fluorophenyl)-6-methoxy-7-nitroisoquinolin-1-amine hydrochloride (676 mg, 1.76 mmol) was mixed with MeOH (25 mL), water (18 mL) and THF (10 mL) under 60° C. oil bath, then ammonium chloride (376 mg, 7.04 mmol) and iron powder, 99% (393 mg, 7.04 mmol) were added. The reaction mixture was stirred under 60° C. oil bath for 90 minutes. Then neutralized with solid NaHCO₃, filtered. The filtrate was concentrated using a rotary evaporator and extracted with EtOAc. The organic layer was dried over MgSO₄, filtered. The filtrate was concentrated and the residue was triturated with ether to give N¹-(3-chloro-4-fluorophenyl)-6-methoxyisoquinoline-1,7-diamine (400 mg) as pale-brown solid. [M+H]⁺=318.38.

### Step 10: N-(1-((3-Chloro-4-fluorophenyl)amino)-6-methoxyisoquinolin-7-yl)-4-(piperidin-1-yl)butanamide

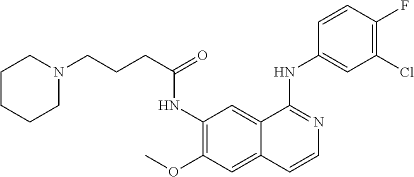 [SMILES: COc1cc2ccnc(Nc3ccc(F)c(Cl)c3)c2cc1NC(=O)CCCN1CCCCC1]

To a solution of N¹-(3-chloro-4-fluorophenyl)-6-methoxyisoquinoline-1,7-diamine (200 mg, 0.629 mmol) with N,N-diisopropylethylamine (163 mg, 1.259 mmol) in THF (10 mL) under ice-water bath, 4-bromobutyryl chloride (140 mg, 0.755 mmol) in DCM (1 mL) was added dropwise. The reaction mixture was stirred at room temperature for an hour. Piperidine (536 mg, 6.29 mmol) and sodium iodide (94 mg, 0.629 mmol) were added and the reaction mixture was stirred at r.t. overnight. The reaction mixture was quenched with brine, and extracted with EtOAc. The organic layer was concentrated with silica gel by using a rotary evaporator. The product was purified by Biotage (12 g), eluted with 50-100% EtOAc in hexanes, then with 5% MeOH in EtOAc. The pure factions were combined and concentrated. The residue was triturated with EtOAc-ether to give N-(1-((3-chloro-4-fluorophenyl)amino)-6-methoxyisoquinolin-7-yl)-4-(piperidin-1-yl)butanamide (112.5 mg) as an off-white solid. Yield=37.9%. [M+H]⁺=471.51. ¹H NMR (500 MHz, DMSO-d₆) δ=9.37 (br s, 1H), 9.16 (s, 1H), 8.80 (br s, 1H), 8.08 (dd, J=2.3, 6.7 Hz, 1H), 7.91 (d, J=5.6 Hz, 1H), 7.73 (td, J=3.6, 8.3 Hz, 1H), 7.39-7.28 (m, 2H), 7.15 (d, J=5.7 Hz, 1H), 3.97 (s, 3H), 2.45 (br t, J=6.8 Hz, 2H), 2.37-2.23 (m, 6H), 1.76 (quin, J=7.1 Hz, 2H), 1.54-1.43 (m, 4H), 1.37 (br d, J=4.5 Hz, 2H).

### Example 2: Preparation of N-(1-((3-chloro-4-fluorophenyl)amino)-6-methoxyisoquinolin-7-yl)-5-(piperidin-1-yl)pentanamide (I-2)

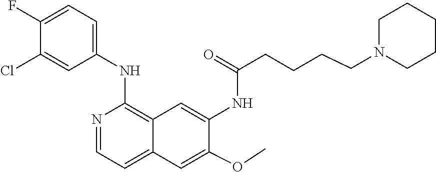 [SMILES: COc1cc2ccnc(Nc3ccc(F)c(Cl)c3)c2cc1NC(=O)CCCCN1CCCCC1]

To a solution of N1-(3-chloro-4-fluorophenyl)-6-methoxyisoquinoline-1,7-diamine (0.1 g, 0.314 mmol) in THF (5 mL), 5-bromopentanoyl chloride (0.075 g, 0.376 mmol), DIPEA (0.085 g, 0.628 mmol) were added, and the mixture was stirred at 0° C. for 2 h. After 2 h, NaI (0.05 g, 0.314 mmol), piperidine (0.27 g, 3.147 mmol) were added and the reaction was stirred at room temperature for 16 h. After completion of reaction, mixture was diluted with brine (20 mL) and extracted with EtOAc (3×10 mL). The combined organic layer was dried over anhydrous Na₂SO₄, concentrated under vacuum to afford the crude. The crude was purified using basic alumina, eluting with 0.8% MeOH in DCM to afford the title compound (0.072 g, 45.9%) as light brown solid. ¹H NMR (400 MHz, DMSO-d₆) δ 9.37 (s, 1H), 9.17 (s, 1H), 8.79 (s, 1H), 8.08 (s, 1H), 7.91 (d, J=4.4 Hz, 1H), 7.73 (s, 1H), 7.33 (s, 2H), 7.15 (d, J=4.8 Hz, 1H), 3.97 (s, 3H), 2.25-2.29 (bs, J=17.6 Hz, 8H), 1.61 (s, 2H), 1.47 (s, 4H), 1.36 (s, 2H). LCMS: [M+2H]⁺=487.3.

### Example 3: Preparation of N-(1-((3-chloro-4-fluorophenyl)amino)-6-methoxyisoquinolin-7-yl)-3-(piperidin-1-yl)propenamide (I-3)

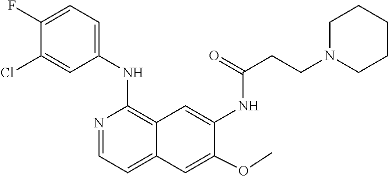 [SMILES: COc1cc2ccnc(Nc3ccc(F)c(Cl)c3)c2cc1NC(=O)CCN1CCCCC1]

To a solution of N¹-(3-chloro-4-fluorophenyl)-6-methoxyisoquinoline-1,7-diamine (0.1 g, 0.314 mmol) in THF (5 mL), 3-bromopropionyl chloride (0.07 g, 0.376 mmol), DIPEA (0.085 g, 0.628 mmol) were added, and the mixture was stirred at 0° C. for 2 h. After 2 h, NaI (0.05 g, 0.314 mmol), piperidine (0.27 g, 3.14 mmol) were added and the mixture stirred at room temperature for 16 h. After completion of the reaction, the mixture was diluted with brine (20 mL) and extracted with EtOAc (3×10 mL). The combined organic layer was dried over anhydrous Na₂SO₄, concentrated under vacuum to afford the crude product which was purified using basic alumina eluting with 0.5% MeOH in DCM to afford the title compound (0.05 g, 34.8%) as light brown solid. ¹H NMR (400 MHz, DMSO-d₆) δ 10.7 (s, 1H), 9.17 (s, 1H), 9.00 (s, 1H), 8.05 (s, 1H), 7.91 (s, 1H), 7.71 (s, 1H), 7.35 (s, 2H), 7.16 (s, 1H), 4.00 (s, 3H), 2.59 (t, J=17.6 Hz, 8H), 1.61 (s, 4H), 1.48 (s, 2H), 1.24 (s, 3H). LCMS: [M+H]⁺=457.2.

### Example 4: Preparation of N-(1-((3-chloro-4-fluorophenyl)amino)-6-methoxyisoquinolin-7-yl)-4-(pyrrolidin-1-yl)butanamide (I-4)

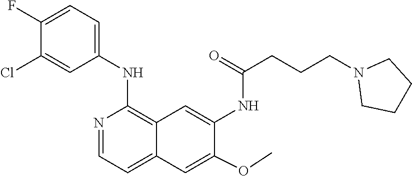 [SMILES: COc1cc2ccnc(Nc3ccc(F)c(Cl)c3)c2cc1NC(=O)CCCN1CCCC1]

To a solution of N¹-(3-chloro-4-fluorophenyl)-6-methoxyisoquinoline-1,7-diamine (0.1 g, 0.314 mmol) in THF (5 mL), 4-bromobutanoyl chloride (0.075 g, 0.376 mmol) and DIPEA (0.085 g, 0.628 mmol) were added and the mixture was stirred at 0° C. for 2 h. After 2 h, NaI (0.05 g, 0.314 mmol) and pyrrolidine (0.23 g, 3.147 mmol) were added and the mixture stirred at room temperature for 16 h. After completion of reaction, mixture was diluted with brine (20 mL) and extracted with EtOAc (3×10 mL). The combined organic layer was dried over anhydrous Na₂SO₄, concentrated under vacuum. The resulting crude product was purified using basic alumina eluting with 1.2% MeOH in DCM to afford the title compound (0.055 g, 38.3%) as light-yellow solid. ¹H NMR (400 MHz, DMSO-d₆) δ 9.45 (s, 1H), 9.19 (s, 1H), 8.83 (s, 1H), 8.09 (d, J=6.4 Hz, 1H), 7.93 (d, J=4.8 Hz, 1H), 7.73 (s, 1H), 7.33-7.37 (t, J=8.8 Hz, 2H), 7.15 (d, J=5.6 Hz, 1H), 3.98 (s, 3H), 2.45 (s, 8H), 1.79 (t, J=8.6 Hz, 2H), 1.69 (s, 4H). LCMS: [M+H]⁺=457.2.

### Example 5: Preparation of N-(1-((3-chloro-4-fluorophenyl)amino)-6-methoxyisoquinolin-7-yl)-4-((2S,6R)-2,6-dimethylmorpholino)butanamide (I-5)

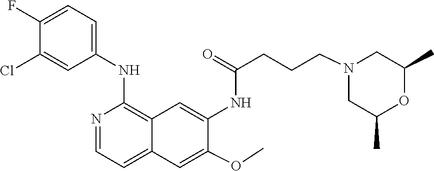 [SMILES: COc1cc2ccnc(Nc3ccc(F)c(Cl)c3)c2cc1NC(=O)CCCN1C[C@@H](C)O[C@@H](C)C1]

To a solution of N¹-(3-chloro-4-fluorophenyl)-6-methoxyisoquinoline-1,7-diamine (0.15 g, 0.471 mmol) in THF (9 mL), 4-bromobutanoyl chloride (0.105 g, 0.566 mmol), DIPEA (0.122 g, 0.944 mmol) were added, and the mixture was stirred at 0° C. for 2 h. After 2 h, NaI (0.071 g, 0.472 mmol) and (2S,6R)-2,6-dimethylmorpholine (0.361 g, 4.712 mmol) were added and the reaction was stirred at room temperature for 16 h. The mixture was diluted with brine (30 mL) and extracted with EtOAc (3×15 mL). The combined organic layers were dried over anhydrous Na₂SO₄, concentrated under vacuum. The resulting crude product was purified using basic alumina to afford the title compound (0.07 g, 29.6%) as an off white solid. ¹H NMR (400 MHz, DMSO-d₆) δ 9.4 (s, 1H), 9.19 (s, 1H), 8.82 (s, 1H), 8.09 (d, J=4.4 Hz, 1H), 7.91 (d, J=6 Hz, 1H), 7.76 (bs, J=8.4 Hz, 1H), 7.35-7.50 (t, J=9.6 Hz, 2H), 7.15 (d, J=5.6 Hz, 1H), 3.98 (s, 3H), 3.54 (s, 2H), 3.76 (d, J=10.8 Hz, 2H), 2.34 (d, J=6.4 Hz, 2H), 1.8 (d, J=6.8 Hz, 2H), 1.60-1.55 (t, J=10.4 Hz, 2H), 1.04 (s 6H). LCMS: [M+2H]⁺=503.3.

### Example 6: Preparation of N-(1-((3-chloro-4-fluorophenyl)amino)-6-methoxyisoquinolin-7-yl)-4-morpholinobutanamide (I-6)

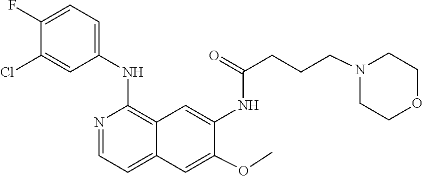 [SMILES: COc1cc2ccnc(Nc3ccc(F)c(Cl)c3)c2cc1NC(=O)CCCN1CCOCC1]

To a solution of N¹-(3-chloro-4-fluorophenyl)-6-methoxyisoquinoline-1,7-diamine (0.12 g, 0.377 mmol) in THF (6 mL), 4-bromobutanoyl chloride (0.085 g, 0.453 mmol), DIPEA (0.1 g, 0.755 mmol) were added, and the mixture was stirred at 0° C. for 2 h. After 2 h, NaI (0.057 g, 0.377 mmol), and morpholine (0.33 g, 3.776 mmol) were added and the mixture stirred at room temperature for 16 h. After completion of the reaction, the mixture was diluted with brine (20 mL) and extracted with EtOAc (3×10 mL). The combined organic layer was dried over anhydrous Na₂SO₄ and concentrated under vacuum to afford the crude product which was purified using flash silica chromatography to give the title compound (0.045 g, 25.2%) as off-white solid. ¹H NMR (400 MHz, DMSO-d₆) δ 9.39 (s, 1H), 9.17 (s, 1H), 8.81 (s, 1H), 8.09 (d, J=4.4 Hz, 1H), 7.91 (d, J=5.6 Hz, 1H), 7.72 (bs, J=4.8 Hz, 1H), 7.35-7.50 (t, J=9.2 Hz, 2H), 7.15 (d, J=5.6 Hz, 1H), 3.96 (s, 3H), 3.56 (s, 4H), 3.16 (d, J=4.8 Hz, 2H), 2.34 (s, 6H), 1.79-1.76 (t, J=6.8 Hz, 2H). LCMS: [M+H]⁺=473.3.

### Example 7: Preparation of N-(1-((3-chloro-4-fluorophenyl)amino)-6-methoxyisoquinolin-7-yl)-4-(4-methylpiperazin-1-yl)butanamide (I-7)

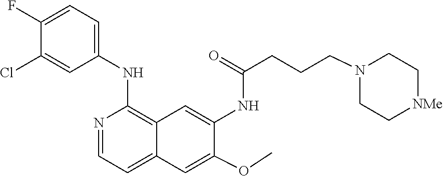 [SMILES: COc1cc2ccnc(Nc3ccc(F)c(Cl)c3)c2cc1NC(=O)CCCN1CCN(C)CC1]

To a solution of N¹-(3-chloro-4-fluorophenyl)-6-methoxyisoquinoline-1,7-diamine (0.1 g, 0.314 mmol) in THF (5 mL), 4-bromobutanoyl chloride (0.075 g, 0.376 mmol), DIPEA (0.085 g, 0.628 mmol) were added and the mixture was stirred at 0° C. for 2 h. After 2 h, NaI (0.05 g, 0.314 mmol), 1-methylpiperizine (0.32 g, 3.147 mmol) were added and the mixture was stirred at room temperature for 16 h. After completion of the reaction, the mixture was diluted with brine (20 mL) and extracted with EtOAc (3×10 mL). The combined organic layer was dried over anhydrous Na₂SO₄, and concentrated under vacuum to afford the crude product which was purified using basic alumina eluting with 1% MeOH in DCM to afford the title compound (0.047 g, 30.7%) as an off white solid. ¹H NMR (400 MHz, DMSO-d₆) δ 9.38 (s, 1H), 9.17 (s, 1H), 8.81 (s, 1H), 8.09 (d, J=5.2 Hz, 1H), 7.91 (d, J=5.2 Hz, 1H), 7.72 (s, 1H), 7.35-7.31 (t, J=10 Hz, 2H), 7.15 (d, J=5.6 Hz, 1H), 3.97 (s, 3H), 2.31 (bs, 12H), 2.12 (s, 3H), 1.77-1.75 (d, J=7.2 Hz, 2H). LCMS: [M+H]⁺=486.2.

### Example 8: Preparation of N-(1-((3-chloro-4-fluorophenyl)amino)-6-methoxyisoquinolin-7-yl)-4-(4,4-difluoropiperidin-1-yl)butanamide (I-8)

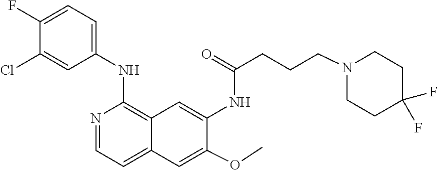 [SMILES: COc1cc2ccnc(Nc3ccc(F)c(Cl)c3)c2cc1NC(=O)CCCN1CCC(F)(F)CC1]

To a solution of N1-(3-chloro-4-fluorophenyl)-6-methoxyisoquinoline-1,7-diamine (0.1 g, 0.314 mmol) in THF (5 mL), 4-bromobutanoyl chloride (0.075 g, 0.376 mmol), DIPEA (0.085 g, 0.628 mmol) were added, and the mixture was stirred at 0° C. for 2 h. After 2 h, NaI (0.05 g, 0.314 mmol) and 4,4-difluoropiperidine (0.9 g, 6.294 mmol) were added and the mixture was stirred at room temperature for 16 h. After completion of reaction, mixture was diluted with brine (20 mL) and extracted with EtOAc (3×10 mL). The combined organic layer was dried over anhydrous Na₂SO₄ and concentrated under vacuum to afford the crude product which was purified using prep HPLC purification to afford the title compound (0.035 g, 11%) as white solid. ¹H NMR (400 MHz, DMSO-d₆) δ 9.38 (s, 1H), 9.16 (s, 1H), 8.81 (s, 1H), 8.09 (d, J=4.8 Hz, 1H), 7.97 (d, J=5.6 Hz, 1H), 7.72 (s, 1H), 7.35-7.50 (t, J=8.8 Hz, 2H), 7.15 (d, J=5.6 Hz, 1H), 3.96 (s, 3H), 2.39 (m, J=6.8 Hz, 8H), 1.96-1.89 (m, J=13.6 Hz, 4H), 1.79-1.76 (t, J=6.8 Hz, 2H). LCMS: [M+H]⁺=507.4.

### Example 9: Preparation of N-(1-((3-fluorophenyl)amino)-6-methoxyisoquinolin-7-yl)-4-(piperidin-1-yl)butanamide (I-9)

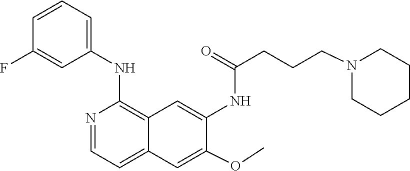 [SMILES: COc1cc2ccnc(Nc3cccc(F)c3)c2cc1NC(=O)CCCN1CCCCC1]

To a degassed solution of N-(1-chloro-6-methoxyisoquinolin-7-yl)-4-(piperidin-1-yl)butanamide (0.15 g, 0.41 mmol), 3-fluoroaniline (50 mg, 0.45 mmol) and t-BuOK (0.093 g, 0.82 mmol) in dioxane (20 V), Xantphos (24 mg, 0.041 mmol) and Pd₂(dba)₃ (38 mg, 0.041 mmol) were added under nitrogen atmosphere and mixture was stirred at 100° C. for 2 h. The mixture was quenched with water (10 mL) and extracted with EtOAc (2×20 mL). The combined organic layers were dried over anhydrous Na₂SO₄ and concentrated under vacuum to afford the crude product which was purified using column chromatography eluting with 12% MeOH in DCM to afford the title compound (52 mg, 28.7%) as light brown solid. ¹H NMR (400 MHz, DMSO-d₆) δ 9.42 (s, 1H), 9.19 (s, 1H), 8.82 (s, 1H), 7.95-7.93 (d, J=5.6 Hz 1H), 7.81-7.78 (d, J=11.6 Hz, 1H), 7.57-7.54 (d, J=8 Hz, 1H), 7.34-7.26 (m, 2H), 7.17-7.16 (d, J=5.2 Hz, 1H), 6.74-6.71 (d, J=14.8 Hz, 1H), 3.973 (s, 3H), 2.38-2.33 (d, J=23.6 Hz, 6H), 1.80 (s, 2H) 1.52 (s, 4H), 1.40 (s, 2H), 1.23 (s, 2H). LCMS: (M+H)⁺=436.53.

### Example 10: Preparation of N-(6-methoxy-1-((3-methoxyphenyl)amino)isoquinolin-7-yl)-4-(piperidin-1-yl)butanamide (I-10)

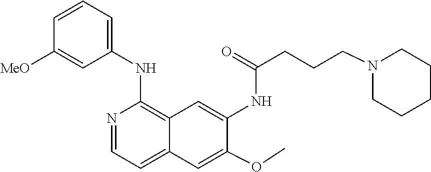 [SMILES: COc1cccc(Nc2nccc3cc(OC)c(NC(=O)CCCN4CCCCC4)cc23)c1]

### Step 1: tert-butyl 4-(piperidin-1-yl)butanoate

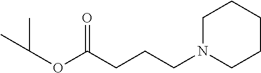

To a solution of tert-butyl 4-bromobutanoate (10 g, 44.8 mmol) in ACN (10V) piperidine (8 mL, 89.6 mmol) was added at RT and the mixture was stirred at 90° C. for 2 h. After completion of the reaction, the mixture was quenched with water (100 mL) and extracted with EtOAc (2×100 mL), organic layer was dried over anhydrous Na₂SO₄, concentrated under vacuum to afford the title compound (10 g, 98.1%) as an off-white solid. The crude material was taken forward to the next step without further purification.

### Step 2: 4-(piperidin-1-yl)butanoic acid

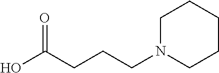 [SMILES: O=C(O)CCCN1CCCCC1]

To a stirred solution of tert-butyl 4-(piperidin-1-yl)butanoate (10 g, 43.8 mmol) in dioxane (100 mL), conc. HCl (1 V) was added at room temperature and the mixture was stirred at 90° C. for 16 h. After completion of the reaction, the mixture was concentrated under vacuum to afford the crude product which was triturated with DCM, solid was filtered and dry to afford the title compound (7.5 g, 99.6%) as an off white solid. ¹H NMR (400 MHz, DMSO-d₆) δ 12.32 (s, 1H), 2.99 (s, 2H), 2.84 (s, 2H), 2.35-2.31 (t, J=13.6 Hz 2H), 1.93-1.92 (d, J=7.2 Hz, 2H), 1.77 (s, 6H), 1.38 (s, 1H).

### Step 3: 1-chloro-6-methoxyisoquinolin-7-amine

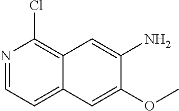

To a stirred solution of 1-chloro-6-methoxy-7-nitroisoquinoline (1 g, 41.8 mmol), MeOH (37 V), water (27 V), and THF (14 V), NH₄Cl (0.81 g, 15.08 mmol) and Fe (0.84 g, 15.08 mmol) was added at 70° C. The mixture was stirred at 70° C. for 5 h and quenched by solid NaHCO₃ (1 g) and the mixture was pass through celite bed and filtrate was concentrated under vacuum to afford the crude product which was stirred in DCM and filtered. The filtrate was concentrated under vacuum to afford the title compound (0.6 g, 68.6%) as yellow solid. LCMS: (M+H)⁺=208.65.

### Step 4: N-(1-chloro-6-methoxyisoquinolin-7-yl)-4-(piperidin-1-yl)butanamide

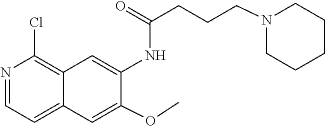 [SMILES: COc1cc2ccnc(Cl)c2cc1NC(=O)CCCN1CCCCC1]

To a stirred solution of 1-chloro-6-methoxyisoquinolin-7-amine (0.6 g, 2.88 mmol) and 4-(piperidin-1-yl)butanoic acid (0.64 g, 3.74 mmol) in DCM (6 mL), DIPEA (1.11 g, 8.64 mmol) and T₃P (50% solution in EtOAc, 1.8 g, 5.76 mmol) was added at RT. The mixture was stirred at 70° C. for 16 h and quenched with water (20 mL) and extracted with EtOAc (3×20 mL). The combined organic layer was dried over anhydrous Na₂SO₄, concentrated under vacuum to afford the crude product which was purified using column chromatography eluting with 10% MeOH in DCM to afford the title compound (0.5 g, 48.05%) as yellow solid. LCMS: (M+H)+=361.87.

### Step 5: N-(6-methoxy-1-((3-methoxyphenyl)amino)isoquinolin-7-yl)-4-(piperidin-1-yl)butanamide

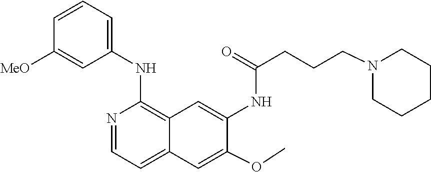 [SMILES: COc1cccc(Nc2nccc3cc(OC)c(NC(=O)CCCN4CCCCC4)cc23)c1]

To a degassed solution of N-(1-chloro-6-methoxyisoquinolin-7-yl)-4-(piperidin-1-yl)butanamide (0.15 g, 0.41 mmol), 3-methoxyaniline (60 mg, 0.49 mmol) and Cs₂CO₃ (0.27 g, 0.82 mmol) in dioxane (10 V), Xantphos (24 mg, 0.041 mmol) and Pd₂(dba)₃ (38 mg, 0.041 mmol) were added under nitrogen atmosphere and mixture was stirred at 100° C. for 2 h. The mixture was quenched with water (10 mL) and extracted with EtOAc (2×20 mL). The combined organic layer was dried over anhydrous Na₂SO₄, concentrated under vacuum to afford the crude product which was purified using column chromatography eluting with 12% MeOH in DCM to afford the title compound (40 mg, 21.5%) as light brown solid. ¹H NMR (400 MHz, DMSO-d₆) δ 9.47 (s, 1H), 8.95 (s, 1H), 8.79 (s, 1H), 7.91 (s, 1H), 7.45 (s, 1H), 7.38-7.37 (d, J=6.8 Hz 2H), 7.17-7.12 (d, J=23.2 Hz, 2H), 6.54 (s, 1H), 3.97 (s, 3H), 3.74 (s, 2H), 2.50 (s, 8H), 1.91 (s, 2H), 1.62 (s, 4H), 1.44 (s, 2H). LCMS: (M+H)⁺=448.57.

### Example 11: Preparation of N-(6-methoxy-1-(m-tolylamino)isoquinolin-7-yl)-4-(piperidin-1-yl)butanamide (I-11)

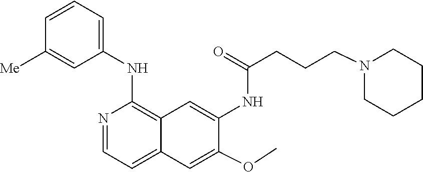 [SMILES: COc1cc2ccnc(Nc3cccc(C)c3)c2cc1NC(=O)CCCN1CCCCC1]

To a degassed solution of N-(1-chloro-6-methoxyisoquinolin-7-yl)-4-(piperidin-1-yl)butanamide (0.15 g, 0.41 mmol), m-toluidine (52 mg, 0.49 mmol) and Cs₂CO₃ (0.27 g, 0.82 mmol) in dioxane (20 V), Xantphos (24 mg, 0.041 mmol) and Pd₂(dba)₃ (38 mg, 0.041 mmol) were added under nitrogen atmosphere and mixture was stirred at 100° C. for 2 h. The mixture was quenched with water (10 mL) and extracted with EtOAc (2×20 mL). The combined organic layers were dried over anhydrous Na₂SO₄ and concentrated under vacuum to afford the crude product which was purified using column chromatography eluting with 12% MeOH in DCM to afford the title compound (50 mg, 27.9%) as a yellow solid. ¹H NMR (400 MHz, DMSO-d₆) δ 9.38 (s, 1H), 8.88 (s, 1H), 8.79 (s, 1H), 7.88-7.871 (d, J=4.8 Hz, 1H), 7.58-7.56 (d, J=8.8 Hz, 2H), 7.30 (s, 1H), 7.18-7.08 (m, 2H), 6.77-6.75 (d, J=6.8 Hz, 1H), 3.96 (s, 3H), 2.46 (s, 8H), 2.29 (s, 3H) 1.79 (s, 2H), 1.52 (s, 4H), 1.39 (s, 2H). LCMS: (M+H)⁺=432.57.

### Example 12: Preparation of N-(1-((4-fluorophenyl)amino)-6-methoxyisoquinolin-7-yl)-4-(piperidin-1-yl)butanamide (I-12)

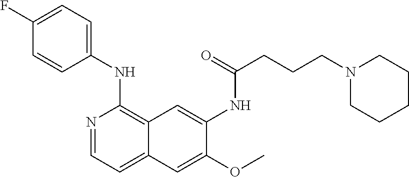 [SMILES: COc1cc2ccnc(Nc3ccc(F)cc3)c2cc1NC(=O)CCCN1CCCCC1]

To a degassed solution of N-(1-chloro-6-methoxyisoquinolin-7-yl)-4-(piperidin-1-yl)butanamide (0.15 g, 0.41 mmol), 4-fluoroaniline (50 mg, 0.45 mmol) and Cs₂CO₃ (0.27 g, 0.82 mmol) in dioxane (20 V), Xantphos (24 mg, 0.041 mmol) and Pd₂(dba)₃ (38 mg, 0.041 mmol) were added under nitrogen atmosphere and mixture was stirred at 100° C. for 2 h. The mixture was quenched with water (10 mL) and extracted with EtOAc (2×20 mL) and combined organic layers were dried over anhydrous Na₂SO₄ and concentrated under vacuum to afford the crude product which was purified using column chromatography eluting with 10% MeOH in DCM to afford the title compound (51 mg, 28.2%) as yellow solid. ¹H NMR (400 MHz, DMSO-d₆) δ 9.40 (s, 1H), 9.01 (s, 1H), 8.79 (s, 1H), 7.85 (s, 1H), 7.75 (s, 2H), 7.30 (s, 1H), 7.12-7.10 (d, J=9.2 Hz, 3H), 3.97 (s, 3H), 2.50 (s, 8H), 1.79 (s, 2H) 1.51 (s, 4H), 1.39 (s, 2H). LCMS: (M+H)⁺=436.53.

### Example 13: Preparation of N-(1-((3-chlorophenyl)amino)-6-methoxyisoquinolin-7-yl)-4-(piperidin-1-yl)butanamide (I-13)

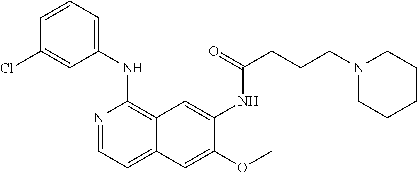 [SMILES: COc1cc2ccnc(Nc3cccc(Cl)c3)c2cc1NC(=O)CCCN1CCCCC1]

To a degassed solution of N-(1-chloro-6-methoxyisoquinolin-7-yl)-4-(piperidin-1-yl)butanamide (0.15 g, 0.41 mmol), 3-chloroaniline (63 mg, 0.45 mmol) and Cs₂CO₃ (0.27 g, 0.82 mmol) in dioxane (20 V), Xantphos (24 mg, 0.041 mmol) and Pd₂(dba)₃ (38 mg, 0.041 mmol) were added under nitrogen atmosphere and mixture was stirred at 100° C. for 2 h. The mixture was quenched by water (10 mL) and extracted with EtOAc (2×20 mL) and organic layer was dried over anhydrous Na₂SO₄, and concentrated under vacuum to afford the crude product which was purified using column chromatography eluting with 8% MeOH in DCM to afford the title compound (50 mg, 26.6%) as yellow solid. ¹H NMR (400 MHz, MeOD) δ 8.80 (s, 1H), 7.88-7.87 (d, J=5.6 Hz, 1H), 7.74 (s, 1H), 7.50-7.78 (d, J=7.6 Hz 1H), 7.40 (s, 1H), 7.29-7.26 (d, J=12 Hz, 1H), 7.18-7.16 (d, J=5.6 Hz, 1H), 7.00-6.99 (d, J=6.4 Hz, 1H), 4.06 (s, 3H), 2.61-2.59 (d, J=6 Hz, 8H), 2.01 (s, 2H), 1.68 (s, 4H), 1.53 (s, 2H). LCMS: (M+H)⁺=452.98.

### Example 14: Preparation of N-(6-methoxy-1-(p-tolylamino)isoquinolin-7-yl)-4-(piperidin-1-yl)butanamide (I-14)

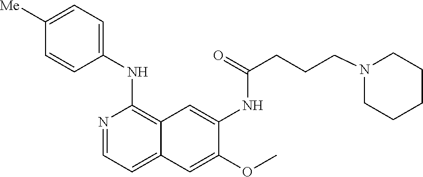 [SMILES: COc1cc2ccnc(Nc3ccc(C)cc3)c2cc1NC(=O)CCCN1CCCCC1]

To a degassed solution of N-(1-chloro-6-methoxyisoquinolin-7-yl)-4-(piperidin-1-yl)butanamide (0.15 g, 0.41 mmol), p-toluidine (53 mg, 0.45 mmol) and Cs₂CO₃ (0.27 g, 0.82 mmol) in dioxane (20 V), Xantphos (24 mg, 0.041 mmol) and Pd₂(dba)₃ (38 mg, 0.041 mmol) were added under nitrogen atmosphere and mixture was stirred at 100° C. for 2 h. The mixture was quenched by water (10 mL) and extracted with EtOAc (2×20 mL) and organic layer was dried over anhydrous Na₂SO₄ and concentrated under vacuum to afford the crude product which was purified using column chromatography eluting with 15% MeOH in DCM to afford the title compound (45 mg, 25.1%) as light yellow solid. ¹H NMR (400 MHz, DMSO-d₆) δ 9.40 (s, 1H), 8.88 (s, 1H), 8.77 (s, 1H), 7.85-7.83 (d, J=5.6 Hz 1H), 7.63-7.61 (d, J=8 Hz, 2H), 7.28 (s, 1H), 7.10-7.04 (m, 3H), 3.96 (s, 3H), 2.33-2.27 (d, J=25.2 Hz, 8H), 1.78 (s, 2H) 1.50 (s, 4H), 1.39 (s, 2H). LCMS: (M+H)⁺=432.57.

### Example 15: Preparation of N-(6-methoxy-1-(phenylamino)isoquinolin-7-yl)-4-(piperidin-1-yl)butanamide (I-15)

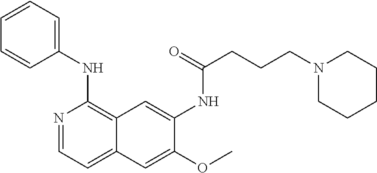 [SMILES: COc1cc2ccnc(Nc3ccccc3)c2cc1NC(=O)CCCN1CCCCC1]

To a degassed solution of N-(1-chloro-6-methoxyisoquinolin-7-yl)-4-(piperidin-1-yl)butanamide (0.15 g, 0.41 mmol), aniline (46 mg, 0.45 mmol) and Cs₂CO₃ (0.27 g, 0.82 mmol) in dioxane (20 V), Xantphos (24 mg, 0.041 mmol) and Pd₂(dba)₃ (38 mg, 0.041 mmol) were added under nitrogen atmosphere and mixture was stirred at 100° C. for 2 h. The mixture was quenched with water (10 mL) and extracted with EtOAc (2×20 ml) and the organic layer was dried over anhydrous Na₂SO₄ and concentrated under vacuum to afford the crude product which was purified using column chromatography eluting with 12% MeOH in DCM to afford the title compound (55 mg, 31.7%) as yellow solid. ¹H NMR (400 MHz, MeOD) δ 8.79 (s, 1H), 7.80-7.78 (d, J=5.6 Hz, 1H), 7.56-7.54 (m, 2H), 7.35-7.31 (m, 2H), 7.27 (s, 1H), 7.11-7.09 (d, J=5.6 Hz, 1H), 7.06-7.01 (m, 1H), 4.06 (s, 3H), 2.62-2.55 (m, 8H), 2.03-1.96 (m, 2H), 1.70-1.64 (m, 4H), 1.53-1.52 (d, J=5.2 Hz, 2H). LCMS: (M+H)+=418.54.

### Example 16: Preparation of N-(1-((4-fluoro-3-(trifluoromethyl)phenyl)amino)-6-methoxyisoquinolin-7-yl)-4-(piperidin-1-yl)butanamide (I-16)

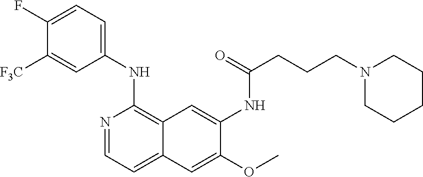 [SMILES: COc1cc2ccnc(Nc3ccc(F)c(C(F)(F)F)c3)c2cc1NC(=O)CCCN1CCCCC1]

To a degassed solution of N-(1-chloro-6-methoxyisoquinolin-7-yl)-4-(piperidin-1-yl)butanamide (0.15 g, 0.41 mmol), 4-fluoro-3-(trifluoromethyl)aniline (89 mg, 0.45 mmol) and Cs₂CO₃ (0.27 g, 0.82 mmol) in dioxane (3 mL), Xantphos (24 mg, 0.041 mmol) and Pd₂(dba)₃ (38 mg, 0.041 mmol) were added under nitrogen atmosphere and mixture was stirred at 100° C. for 2 h. After completion of the reaction, the mixture was diluted with water (20 mL) and extracted with EtOAc (2×30 ml) and organic layer was dried over anhydrous Na₂SO₄ and concentrated to afford the crude product which was purified using column chromatography eluting with 12% MeOH in DCM to afford the title compound (65 mg, 31.1%) as yellow solid. ¹H NMR (400 MHz, Methanol-d₄) δ, 8.80 (s, 1H), 7.80-7.94 (m, 1H), 7.90-7.84 (m, 2H), 7.28-7.23 (m, 2H), 7.15-7.13 (d, J=5.6 Hz, 1H), 4.05 (s, 3H), 2.59-2.49 (m, 8H), 2.02-1.94 (m, 2H), 1.67-1.62 (m, 4H), 1.51 (bs, 2H). LCMS: (M+H)⁺=505.2.

### Example 17: Preparation of N-(1-((3-chloro-4-fluorophenyl)amino)-6-methylisoquinolin-7-yl)-4-(piperidin-1-yl)butanamide (I-17)

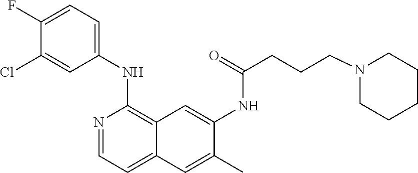 [SMILES: Cc1cc2ccnc(Nc3ccc(F)c(Cl)c3)c2cc1NC(=O)CCCN1CCCCC1]

### Step 1: 4-methyl-3-nitro-N-(pivaloyloxy)benzamide

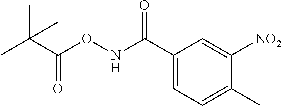 [SMILES: Cc1ccc(C(=O)NOC(=O)C(C)(C)C)cc1[N+](=O)[O-]]

To a cooled solution of 4-methyl-3-nitrobenzoic acid (5 g, 27.6 mmol) in DCM (50 mL), oxalyl chloride (2.5 mL, 30.36 mmol) and DMF (1 mL) were added at 0° C. and the mixture was stirred at room temperature for 3 h. The mixture was concentrated under N₂ atmosphere to afford 4-methyl-3-nitrobenzoyl chloride. To a cooled solution of 0-pivaloylhydroxylammonium trifluoromethanesulfonate (2.5 g, 16.77 mmol) and NaHCO₃ (6.95 g, 82.8 mmol) in EtOAc:water (2:1, 50 mL) was the added 4-methyl-3-nitrobenzoyl chloride in EtOAc (5 mL) at 0° C. and the mixture was stirred at room temperature for 3 h. After completion of the reaction, the mixture was diluted with water (100 mL) and extracted in EtOAc (3×150 mL). The combined organic was dried over anhydrous Na₂SO₄ and concentrated under vacuum to afford the title compound (3 g, 38.8%) as an off white solid. ¹H NMR (400 MHz, DMSO-d₆): δ 8.83 (s, 1H), 8.44 (s, 1H), 8.07 (d, J 6.4 Hz, 1H), 7.60 (d, 8 Hz, 1H), 4.50 (t, J=5.2 Hz, 1H), 3.37-3.22 (m, 4H), 2.48 (s, 5H).

### Step 2: 6-methyl-7-nitroisoquinolin-1(2H)-one

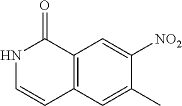

To a stirred solution of 4-methyl-3-nitro-N-(pivaloyloxy)benzamide (3 g, 10.7 mmol) in MeOH (30 mL), vinyl acetate (1.17 g, 13.6 mmol), cesium acetate (0.52 g, 2.72 mmol) and pentamethylcyclopentadienyl rhodium dichloride dimer (50 mg, 0.9 mmol) were added under nitrogen atmosphere and the mixture was stirred at 70° C. for 4 h. The mixture was concentrated under vacuum to afford the crude product which was triturate with 5% MeOH in diethyl ether, filtered and dry to afford the title compound (1.7 g, 77.8%) as brown solid. LCMS: (M+H)⁺=205.15.

### Step 3: 1-chloro-6-methyl-7-nitroisoquinoline

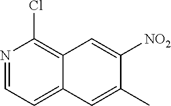

6-Methyl-7-nitroisoquinolin-1(2H)-one (0.250 g, 1.22 mmol) was mixed with phosphorous (V) oxychloride (5 mL) and the reaction mixture was stirred under 130° C. 16 h. The mixture concentrated under vacuum and the excess of POCl₃ was stripped off with toluene. The residue was diluted with EtOAc (50 mL) and ice cooled 10% solution of NaHCO₃ (100 mL). The layers were separated and the organic layer was dried over anhydrous Na₂SO₄ and concentrated under vacuum to afford crude product which was triturated with diethyl ether to afford the title compound (0.25 g, 91.7%) as pale-yellow solid. LCMS: (M+H)⁺=223.14.

### Step 4: 1-chloro-6-methylisoquinolin-7-amine

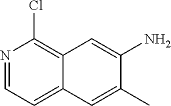

To a solution of 1-chloro-6-methyl-7-nitroisoquinoline (250 mg, 1.12 mmol) in MeOH (8 mL), water (6 mL) and THF (3 mL) at 60° C., ammonium chloride (180 mg, 3.243 mmol) and iron powder, 99% (181 mg, 3.243 mmol) were added. The mixture was stirred at 60° C. for 4 h. The mixture was neutralized with solid NaHCO₃, filtered. The organic layer of filtrate was evaporated and extracted with EtOAc (2×20 mL). The organic layer was dried over Na₂SO₄, concentrated and triturated with ether to afford the title compound (180 mg, 83.2%) as brown solid. LCMS: (M+H)⁺=193.12.

### Step 5: N-(1-chloro-6-methylisoquinolin-7-yl)-4-(piperidin-1-yl)butanamide

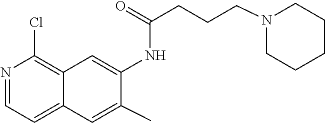 [SMILES: Cc1cc2ccnc(Cl)c2cc1NC(=O)CCCN1CCCCC1]

To a solution of 1-chloro-6-methylisoquinolin-7-amine (0.15 g, 0.778 mmol) in DCM (1.5 mL), 4-(piperidin-1-yl)butanoic acid (0.15 g, 0.875 mmol), N,N-Diisopropylethylamine (1.5 mL) and 50% T3P in EtOAc (0.64 g, 2.02 mmol) were added and the mixture was stirred at 60° C. for 6 h. The mixture was concentrated and purified using silica gel chromatography. The product was eluted with 10% MeOH in MDC and the resulting product was triturated with ether to give title compound (200 mg, 74.3%) as a light brown solid. LCMS [M+H]⁺=346.31.

### Step 6: N-(1-((3-chloro-4-fluorophenyl)amino)-6-methylisoquinolin-7-yl)-4-(piperidin-1-yl)butanamide

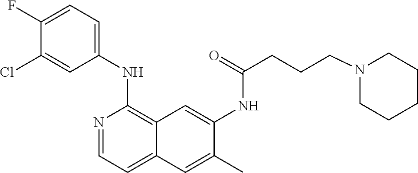 [SMILES: Cc1cc2ccnc(Nc3ccc(F)c(Cl)c3)c2cc1NC(=O)CCCN1CCCCC1]

To a stirred solution of N-(1-chloro-6-methylisoquinolin-7-yl)-4-(piperidin-1-yl) butanamide (200 mg, 0.578 mmol) and 3-chloro-4-fluoroaniline (100 mg, 0.687 mmol) in 1,4-dioxane (2 mL), 4.0 M hydrochloric acid in dioxane (0.041 g, 1.556 mmol) was added. The mixture was stirred at 100° C. for 6 h. The mixture was concentrated under vacuum to afford the title compound (80 mg, 30.4%) as light brown solid. ¹H NMR (400 MHz, Methanol-d₄): δ 8.82 (s, 1H), 7.98 (s, 1H), 7.85 (d, J=7.6 Hz, 1H), 7.59 (d, J=6.8 Hz, 2H), 7.55-7.53 (m, 1H), 7.39 (d, J=6 Hz, 1H), 3.66 (d, J=11.6 Hz, 2H), 3.39-3.28 (m, 2H), 3.06 (t, J=12.8 Hz, 2H), 2.82-2.78 (m, 2H), 2.63 (s, 3H), 2.25-2.24 (m, 2H), 2.04 (d, J=14.8, 2H), 1.94-1.84 (m, 4H), 1.37 (s, 1H). LCMS: (M+H)⁺=455.2.

### Example 18: Preparation of N-(1-((3-chloro-4-fluorophenyl)amino)isoquinolin-7-yl)-4-(piperidin-1-yl)butanamide (I-18)

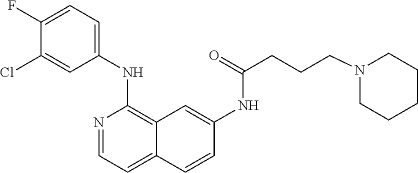 [SMILES: O=C(CCCN1CCCCC1)Nc1ccc2ccnc(Nc3ccc(F)c(Cl)c3)c2c1]

### Step 1: 3-nitro-N-(pivaloyloxy)benzamide

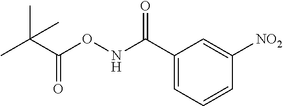 [SMILES: CC(C)(C)C(=O)ONC(=O)c1cccc([N+](=O)[O-])c1]

To a cooled solution of 3-nitrobenzoic acid (2.2 g, 13.6 mmol) in DCM (22 mL) and DMF (cat.), oxalyl chloride (1.3 ml, 14.48 mmol) was added at 0° C. and the mixture was stirred at room temperature for 3 h. The mixture was concentrated under N₂ atmosphere to afford 3-nitrobenzoyl chloride. To a cooled solution of O-pivaloylhydroxylammonium trifluoromethanesulfonate (3.8 g, 14.48 mmol) and NaHCO₃ (3.3 g, 39.49 mmol) in EtOAc:H₂O (12:1, 50 mL), was added the 3-nitrobenzoyl chloride in EtOAc (10 mL) at 0° C. and the mixture was stirred at room temperature for 3 h. The mixture was diluted with water (100 mL) and extracted in EtOAc (3×150 mL). The combined organic was dried over anhydrous Na₂SO₄ and concentrated under vacuum to afford the title compound (1.7 g, 48.5%) as an off white solid. The crude material was taken forward to the next step without further purification. LCMS: (M+H)+=266.25.

### Step 2: 7-nitroisoquinolin-1(2H)-one

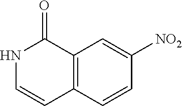 [SMILES: O=c1[nH]ccc2ccc([N+](=O)[O-])cc12]

To a stirred solution of 3-nitro-N-(pivaloyloxy)benzamide (1 g, 3.75 mmol) in MeOH (10 mL), was added vinyl acetate (0.48 g, 4.87 mmol), cesium acetate (0.25 g, 1.323 mmol) and pentamethylcyclopentadienyl rhodium dichloride dimer (116 mg, 0.37 mmol) under nitrogen atmosphere. The mixture was then stirred at 70° C. for 16 h and concentrated under vacuum to afford the crude product which was triturated with 1% MeOH in diethyl ether to afford the title compound (0.7 g, 98.0%) as brown solid. The crude material was taken forward to the next step without further purification.

### Step 3: 1-chloro-7-nitroisoquinoline

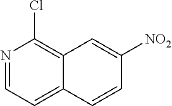 [SMILES: O=[N+]([O-])c1ccc2ccnc(Cl)c2c1]

A suspension of 7-nitroisoquinolin-1(2H)-one (0.7 g, 3.68 mmol) with POCl₃ (10 mL) was stirred at 100° C. for 2 h. After completion of the reaction, the mixture was concentrated and the residue was diluted with DCM (50 mL) and ice cooled water (50 mL), neutralized with solid NaHCO₃ and extracted with DCM (2×50 mL). The combined organic layers were concentrated and triturated with diethyl ether to give 1-chloro-7-nitroisoquinoline (0.4 g, 1.92 mmol, 52.1%) as pale-yellow solid. LCMS: (M−H)⁺=208.60.

### Step 4: 1-chloroisoquinolin-7-amine

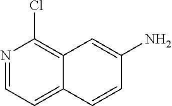 [SMILES: Nc1ccc2ccnc(Cl)c2c1]

To a stirred solution of 1-chloro-7-nitroisoquinoline (0.4 g, 1.92 mmol) in MeOH (37 V), water (27 V), and THF (14 V), NH₄Cl (0.37 g, 6.92 mmol) and Fe (0.38 g, 6.92 mmol) were added at 60° C. and the mixture was stirred at 60° C. for 5 h. After completion of reaction, the mixture was pass through celite bed and filtrate was concentrated under vacuum to afford the crude product which was extracted with DCM (2×30 mL), dried over anhydrous Na₂SO₄ and concentrated to give the title compound (0.24 g, 70.1%) as yellow solid. LCMS: (M+H)⁺=178.62.

### Step 5: N-(1-chloroisoquinolin-7-yl)-4-(piperidin-1-yl)butanamide

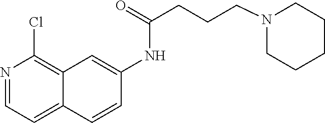 [SMILES: O=C(CCCN1CCCCC1)Nc1ccc2ccnc(Cl)c2c1]

To a stirred solution of 1-chloroisoquinolin-7-amine (0.15 g, 0.84 mmol) and 4-(piperidin-1-yl)butanoic acid (0.22 g, 1.26 mmol) in DCM (10 V), DIPEA (0.32 g, 2.52 mmol) and T₃P (50% solution, 0.5 g, 1.68 mmol) was added at RT. The mixture was stirred at 70° C. for 16 h. After completion of reaction, the mixture was diluted with water (20 mL) and extracted with EtOAc (3×20 mL) and organic layer was dried over anhydrous Na₂SO₄, concentrated under vacuum to afford the crude product which was purified using column chromatography, eluting with 8-11% MeOH in DCM to afford the title compound (0.1 g, 35.9%) as yellow solid. LCMS: (M+H)⁺=331.84.

### Step 6: N-(1-((3-chloro-4-fluorophenyl)amino)isoquinolin-7-yl)-4-(piperidin-1-yl)butanamide

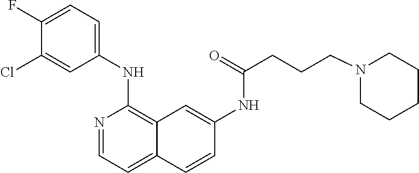 [SMILES: O=C(CCCN1CCCCC1)Nc1ccc2ccnc(Nc3ccc(F)c(Cl)c3)c2c1]

To a degassed solution of N-(1-chloroisoquinolin-7-yl)-4-(piperidin-1-yl)butanamide (0.1 g, 0.301 mmol), 3-chloro-4-fluoroaniline (52.64 mg, 0.36 mmol) and Cs₂CO₃ (0.19 g, 0.6 mmol) in dioxane (10 V), Xantphos (17.45 mg, 0.030 mmol) and Pd₂(dba)₃ (27.6 mg, 0.030 mmol) were added under nitrogen atmosphere and mixture was stirred at 100° C. for 2 h. After completion of reaction, the mixture was diluted with water (20 mL) and extracted with EtOAc (2×20 mL) and organic layer was dried over anhydrous Na₂SO₄, concentrated to afford the crude product which was purified using column chromatography, eluting with 12% MeOH in DCM to afford the title compound (65 mg, 48.9%) as an off white solid. ¹H NMR (400 MHz, Methanol d₄) δ 8.59 (s, 1H), 7.90-7.86 (m, 2H), 7.81-7.79 (d, J=8.8 Hz, 1H), 7.72-7.69 (dd, J=2 Hz, J=2 Hz, 1H), 7.57-7.53 (m, 1H), 7.23-7.17 (m, 3H), 3.27-3.106 (m, 6H), 2.67-2.64 (t, J=13.6, 2H), 2.18-2.11 (m, 2H), 1.86 (s, 4H), 1.68 (s, 2H), 1.30 (s, 1H). LCMS: (M+H)⁺=440.95.

### Example 19: Preparation of N-(1-((2-chloro-4-fluorophenyl)amino)-6-methoxyisoquinolin-7-yl)-4-(piperidin-1-yl)butanamide (I-19)

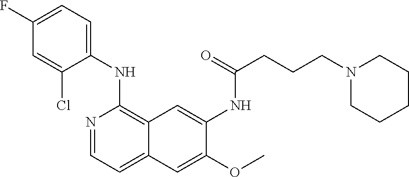 [SMILES: COc1cc2ccnc(Nc3ccc(F)cc3Cl)c2cc1NC(=O)CCCN1CCCCC1]

To a solution of N-(1-chloro-6-methoxyisoquinolin-7-yl)-4-(piperidin-1-yl)butanamide (100 mg, 0.276 mmol) in dioxane (2 mL) Cs₂CO₃ (179 mg, 0.552 mmol), 2-chloro-4-fluoroaniline (44 mg, 0.304 mmol) were added and mixture was degassed with N₂ for 10 min. To this, Xantphos (15 mg, 0.027 mmol) and Pd₂(dba)₃ (25 mg, 0.276 mmol) were added and mixture was stirred at 100° C. for 2 h. The mixture was concentrated and the residue was purified by silica gel chromatography eluting with 5-20% MeOH/DCM to afford the title compound (25 mg, 26.9%) as light brown solid. ¹H NMR (400 MHz, MeOD-d₄): δ 9.44 (s, 1H), 8.87 (s, 1H), 8.66 (s, 1H), 7.79-7.77 (d, J=5.2 Hz, 1H), 7.71-7.67 (d, J=6 Hz, 1H), 7.50-7.48 (d, J=6.4 Hz, 1H), 7.39 (s, 1H), 7.33 (s, 1H), 7.26-7.18 (m, 1H), 7.09-7.08 (d, J=5.2 Hz, 1H), 4.21 (s, 2H), 2.51 (s, 8H), 1.87 (s, 2H) 1.57 (s, 4H). 1.24 (s, 2H); LCMS: (M+1)⁺=470.97.

### Example 20: Preparation of N-(1-((4-fluoro-3-methylphenyl)amino)-6-methoxyisoquinolin-7-yl)-4-(piperidin-1-yl)butanamide (I-20)

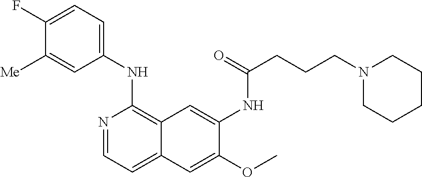 [SMILES: COc1cc2ccnc(Nc3ccc(F)c(C)c3)c2cc1NC(=O)CCCN1CCCCC1]

To a solution of N-(1-choro-6-methoxyisoquinolin-7-yl)-4-(piperidin-1-yl) butanamide (100 mg, 0.276 mmol) in dioxane (2 mL) Cs₂CO₃ (180 mg, 0.552 mmol), 4-fluoro-3-methylaniline (38 mg, 0.304 mmol) were added and mixture was degassed with N₂ for 10 min. To this, xantphos (16 mg, 0.027 mmol) and Pd₂(dba)₃ (25 mg, 0.276 mmol) were added and mixture was stirred at 100° C. for 2 h. The mixture was concentrated and the residue was purified using silica gel chromatography eluting with 5-20% MeOH/DCM to afford the title compound (35 mg, 28.1%) as grey fluffy solid. ¹H NMR (400 MHz, MeOD-d₄): δ 9.364 (s, 1H), 8.92 (s, 1H), 8.79 (s, 1H), 7.87-7.85 (d, J=5.6 Hz 1H), 7.63-7.61 (d, J=7.2 Hz, 2H), 7.30 (s, 1H) 7.12-7.03 (m, 1H), 3.97 (s, 3H) 2.32-2.30 (d, J=6 Hz, 6H), 2.24 (s, 3H), 1.77 (s, 2H), 1.50 (s, 4H) 1.31 (s, 1H); LCMS: (M+1)⁺=470.97.

### Example 21: Preparation of N-(1-((3-chloro-4-methylphenyl)amino)-6-methoxyisoquinolin-7-yl)-4-(piperidin-1-yl)butanamide (I-21)

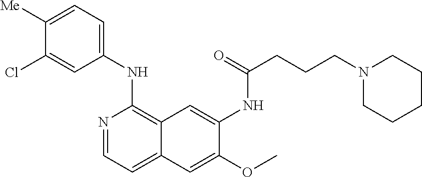 [SMILES: COc1cc2ccnc(Nc3ccc(C)c(Cl)c3)c2cc1NC(=O)CCCN1CCCCC1]

To a degassed solution of N-(1-chloro-6-methoxyisoquinolin-7-yl)-4-(piperidin-1-yl)butanamide (0.15 g, 0.41 mmol), 3-chloro-4-methylaniline (70 mg, 0.45 mmol) and Cs₂CO₃ (0.27 g, 0.82 mmol) in dioxane (3 mL), Xantphos (24 mg, 0.041 mmol) and Pd₂(dba)₃ (38 mg, 0.041 mmol) were added under nitrogen atmosphere and mixture was stirred at 100° C. for 2 h. The mixture was diluted with water (25 mL) and extracted with EtOAc (2×30 mL). The combined organic layers were dried over anhydrous Na₂SO₄, concentrated to afford the crude product which was purified using column chromatography eluting with 12% MeOH in DCM to afford the title compound (65 mg, 33.6%) as yellow solid. ¹H NMR (400 MHz, Methanol-d₄) δ 8.767 (s, 1H), 7.84-7.82 (d, J=6 Hz, 1H), 7.70-7.70 (d, J=2 Hz, 1H), 7.41-7.38 (dd, J=2.4 Hz, J=2 Hz, 1H), 7.26 (s, 1H), 7.23-7.21 (d, J=8 Hz, 1H), 7.120-7.11 (d, J=5.6 Hz, 1H), 4.05 (s, 3H), 2.58-2.48 (m, 8H), 2.02-1.96 (m, 2H), 1.67-1.62 (m, 4H), 1.51-1.50 (d, J=4.4 Hz, 2H); LCMS: (M+H)⁺=567.01.

### Example 22: Preparation of N-(1-((4-fluoro-3-methoxyphenyl)amino)-6-methoxyisoquinolin-7-yl)-4-(piperidin-1-yl)butanamide (I-22)

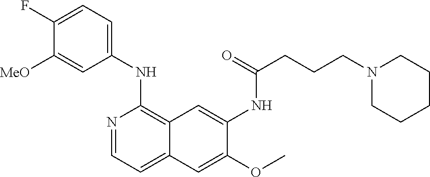 [SMILES: COc1cc(Nc2nccc3cc(OC)c(NC(=O)CCCN4CCCCC4)cc23)ccc1F]

To a solution of N-(1-chloro-6-methoxyisoquinolin-7-yl)-4-(piperidin-1-yl)butanamide (100 mg, 0.276 mmol) in dioxane (2 mL) Cs₂CO₃ (180 mg, 0.552 mmol), 4-fluoro-3-methoxyaniline (42 mg, 0.304 mmol) were added and mixture was degassed with N₂ for 10 min. To this, Xantphos (16 mg, 0.027 mmol) and Pd₂(dba)₃ (25 mg, 0.276 mmol) were added and the mixture was stirred at 100° C. for 2 h. The mixture was concentrated, the residue was purified by silica gel chromatography and product was eluted with 12-20% MeOH/DCM to afford the title compound (35 mg, 27.2%) as yellow solid. ¹H NMR (400 MHz, MeOD-d₄): δ 9.38 (s, 1H), 8.96 (s, 1H), 8.78 (s, 1H), 7.88-7.87 (d, J=5.6 Hz 1H), 7.61-7.59 (t, J=6 Hz, 2H), 7.42-7.40 (t, J=8.8 Hz, 1H) 7.30 (s, 1H), 7.13-7.08 (m, 2H) 3.96 (s, 3H), 3.82 (s, 3H), 2.50-2.44 (t, J=22 Hz, 3H), 2.31-2.27 (m, J=13.6 Hz, 6H) 1.80-1.74 (m, 2H) 1.48-1.47 (d, J=4.8 Hz, 4H), 1.37 (s, 2H); LCMS: (M+H)⁺=467.5.

### Example 23: Preparation of N-(1-((3,5-dichloro-4-fluorophenyl)amino)-6-methoxyisoquinolin-7-yl)-4-(piperidin-1-yl)butanamide (I-23)

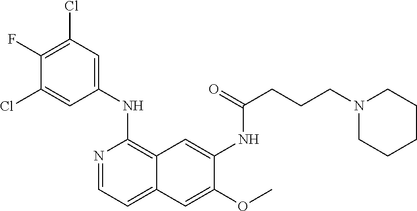 [SMILES: COc1cc2ccnc(Nc3cc(Cl)c(F)c(Cl)c3)c2cc1NC(=O)CCCN1CCCCC1]

To a solution of N-(1-chloro-6-methoxyisoquinolin-7-yl)-4-(piperidin-1-yl) butanamide (100 mg, 0.276 mmol) in dioxane (2 mL) Cs₂CO₃ (180 mg, 0.552 mmol), 3,5-dichloro-4-fluoroaniline (54 mg, 0.304 mmol) were added and mixture was degassed with N₂ for 10 min. To this, Xantphos (15 mg, 0.027 mmol) and Pd₂(dba)₃ (25 mg, 0.276 mmol) were added and mixture was stirred at 100° C. for 2 h. The mixture was concentrated and the residue was purified by silica gel chromatography eluting with 5-20% MeOH/DCM to afford the title compound (60 mg, 43.0%) as light brown solid. ¹H NMR (400 MHz, Methanol-d₄): δ 8.79 (s, 1H), 7.90-7.89 (d, J=5.6 Hz 1H), 7.76-7.75 (d, J=6 Hz, 2H), 7.27 (s, 1H), 7.18-7.16 (d, J=6 Hz, 1H), 4.06 (s, 3H) 2.60-2.53 (m, 8H) 2.01-1.98 (t, J=15.2 Hz, 2H), 1.69-1.64 (m, 4H), 1.53-1.51 (d, J=4.8 Hz, 2H). LCMS: (M+1)⁺=505.42.

### Example 24: Preparation of N-(1-((3-chloro-4-methoxyphenyl)amino)-6-methoxyisoquinolin-7-yl)-4-(piperidin-1-yl)butanamide (I-24)

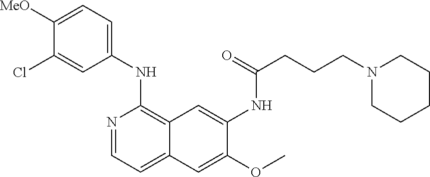 [SMILES: COc1ccc(Nc2nccc3cc(OC)c(NC(=O)CCCN4CCCCC4)cc23)cc1Cl]

To a solution of N-(1-chloro-6-methoxyisoquinolin-7-yl)-4-(piperidin-1-yl)butanamide (100 mg, 0.276 mmol) in dioxane (2 mL) Cs₂CO₃ (180 mg, 0.552 mmol), 3-chloro-4-methoxyaniline (46 mg, 0.304 mmol) were added and mixture was degassed with N₂ for 10 min. To this mixture, Xantphos (15 mg, 0.027 mmol) and Pd₂(dba)₃ (25 mg, 0.276 mmol) were added and mixture was stirred at 100° C. for 2 h. The mixture was evaporated and the residue was purified by silica gel chromatography and product was eluted with 5-20% MeOH/DCM to get title compound (35 mg, 27.2%) as yellow solid. ¹H NMR (400 MHz, MeOD-d₄): δ 8.77 (s, 1H), 7.79-7.77 (d, J=5.6 Hz 1H), 7.65-7.64 (d, J=2.4 Hz, 1H), 7.44-7.42 (m, 1H), 7.26 (s, 1H), 7.08-7.05 (m, 2H), 4.09-7.05 (d, J=14 Hz, 3H) 3.89 (s, 3H), 2.74-2.67 (m, 6H), 2.62-2.59 (t, J=14.4 Hz, 2H) 2.06-1.99 (m, 2H) 1.73-1.70 (t, J=11.6 Hz, 4H), 1.56 (s, 2H), 1.33 (s, 2H); LCMS: (M+H)⁺=483.5.

### Example 25: Preparation of N-(1-((3-chloro-5-fluorophenyl)amino)-6-methoxyisoquinolin-7-yl)-4-(piperidin-1-yl)butanamide (I-25)

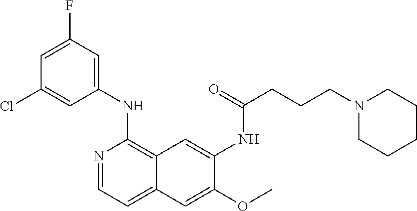 [SMILES: COc1cc2ccnc(Nc3cc(F)cc(Cl)c3)c2cc1NC(=O)CCCN1CCCCC1]

To a solution of N-(1-chloro-6-methoxyisoquinolin-7-yl)-4-(piperidin-1-yl)butanamide (100 mg, 0.276 mmol) in dioxane (2 mL), Cs₂CO₃ (180 mg, 0.552 mmol) and 3-chloro-5-fluoroaniline (46 mg, 0.304 mmol) were added and mixture was degassed with N₂ for 10 min. To this mixture, Xantphos (15 mg, 0.027 mmol) and Pd₂(dba)₃ (25 mg, 0.276 mmol) were added and the mixture was stirred at 100° C. for 2 h. The mixture was concentrated and the residue was purified by silica gel chromatography and eluted with 10-20% MeOH/DCM to afford the title compound (35 mg, 26.9%) as white solid. ¹H NMR (400 MHz, MeOD-d₄): δ 8.81 (s, 1H), 7.96-7.94 (d, J=5.6 Hz 1H), 7.52-7.51 (d, J=4 Hz, 1H), 7.49 (s, 1H), 7.31 (s, 1H), 7.23-7.23 (d, J=5.6 Hz, 2H), 6.77-6.75 (d, J=8.4 Hz) 4.07 (s, 3H) 2.78-2.71 (m, 3H), 2.64-2.61 (t, J=14 Hz, 6H), 2.06-2.02 (t, J=16 Hz, 2H) 1.74-1.71 (t, J=11.2 Hz, 4H), 1.57 (s, 2H), 1.30 (s, 1H); LCMS: (M+H)⁺=471.49.

### Example 26: Preparation of N-(6-chloro-1-((3-chloro-4-fluorophenyl)amino)isoquinolin-7-yl)-4-(piperidin-1-yl)butanamide (I-26)

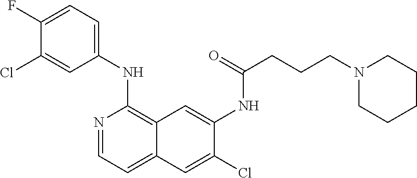 [SMILES: O=C(CCCN1CCCCC1)Nc1cc2c(Nc3ccc(F)c(Cl)c3)nccc2cc1Cl]

### Step 1: 4-chloro-3-nitro-N-(pivaloyloxy)benzamide

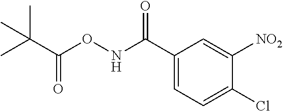 [SMILES: CC(C)(C)C(=O)ONC(=O)c1ccc(Cl)c([N+](=O)[O-])c1]

To a solution of 4-chloro-3-nitrobenzoic acid (1 g, 4.96 mmol) in DCM (10 mL), was added oxalyl chloride (4 mL, 5.449 mmol) and DMF (cat.) at 0° C. and the mixture was stirred at RT for 2 h. After 2 h, the mixture was concentrated. To a solution of O-pivaloylhydroxyl ammonium-trifluoromethane sulfonate (0.8 g, 6.770 mmol) and NaHCO₃ (0.83 g, 9.523 mmol) in EtOAc (10 ml) and water (10 mL), the above prepared acid chloride was added dropwise and the mixture was stirred at RT for 4 h. The mixture was quenched with saturated NHCO₃ solution (20 mL) and extracted with EtOAc (200 mL). The organic layer was dried over anhydrous Na₂SO₄ and concentrated under vacuum to afford the title compound (1.3 g, 87.1%) as an off white solid. The crude material was taken forward by pentane trituration to the next step. LCMS: (M+H)⁺=301.

### Step 2: 6-chloro-7-nitroisoquinolin-1(2H)-one

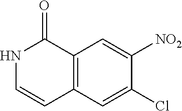 [SMILES: O=c1[nH]ccc2cc(Cl)c([N+](=O)[O-])cc12]

To a stirred solution of 4-chloro-3-nitro-N-(pivaloyloxy)benzamide (1 g, 3.325 mmol) in MeOH (10 mL), vinyl acetate (0.42 g, 4.87 mmol), cesium acetate (0.25 g, 1.323 mmol) and pentamethylcyclopentadienyl rhodium dichloride dimer (40 mg, 0.06 mmol) were added under nitrogen and the mixture was stirred at 70° C. for 16 h. The mixture was concentrated under vacuum to afford the crude product which was triturated with 1% MeOH in diethyl ether. The resulting was filtered and dried over Na₂SO₄ to afford the title compound (0.35 g, 46.9%) as brown solid. LCMS: (M+H)⁺=225.2.

### Step 3: 1,6-dichloro-7-nitroisoquinoline

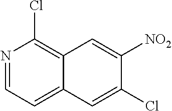 [SMILES: O=[N+]([O-])c1cc2c(Cl)nccc2cc1Cl]

6-Chloro-7-nitroisoquinolin-1(2H)-one (0.3 g, 1.335 mmol) was mixed with phosphorous oxychloride (6 mL) and the mixture was stirred at 100° C. for 1 h. The mixture was concentrated and the traces of POCl₃ was removed by stripping with toluene. The residue was diluted with ice cold water (50 mL) and extracted with DCM (2×20 mL), and back washed with saturated NaHCO₃ solution. The organic layer was concentrated and triturated with diethyl ether to give 1-chloro-6-methoxy-7-nitroisoquinoline (0.23 g, 1.05 mmol, 78.24%) as pale-yellow solid which was used as is in next step.

### Step 4: 6-chloro-N-(3-chloro-4-fluorophenyl)-7-nitroisoquinolin-1-amine

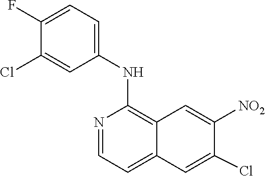 [SMILES: O=[N+]([O-])c1cc2c(Nc3ccc(F)c(Cl)c3)nccc2cc1Cl]

To a suspension of 1,6-dichloro-7-nitroisoquinoline (300 mg, 1.23 mmol) and 3-chloro-4-fluoroaniline (185 mg, 1.27 mmol) in 1,4-dioxane (5 mL), 4M hydrochloric acid in dioxane (0.6 mL, 2.46 mmol) was added and the mixture was stirred at 100° C. for 2 h. The mixture was diluted with diethyl ether and the resulting precipitate was filtered. The solid was rinsed with ether to give the title compound (300 mg, 69.0%) as a brown solid. LCMS: (M+H)⁺=354.2.

### Step 5: 6-chloro-N1-(3-chloro-4-fluorophenyl)isoquinoline-1,7-diamine

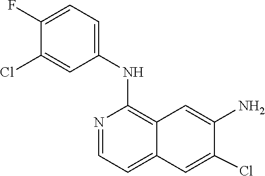 [SMILES: Nc1cc2c(Nc3ccc(F)c(Cl)c3)nccc2cc1Cl]

To a solution of 6-chloro-N-(3-chloro-4-fluorophenyl)-7-nitroisoquinolin-1-amine (320 mg, 0.908 mmol) in MeOH (12 mL), water (9 mL) and THF (12 mL), ammonium chloride (179 mg, 3.346 mmol) and iron powder (182 mg, 2.542 mmol) were added to the mixture. The mixture was stirred at 60° C. for 90 min and mixture was filtered, concentrated and extracted with EtOAc (2×20 mL). The organic layer was dried over Na₂SO₄, concentrated and the residue was triturated with ether to give the title compound (220 mg, 0.679 mmol, 75.2%) as a brown solid. LCMS [M+H]⁺=324.2.

### Step 6: N-(6-chloro-1-((3-chloro-4-fluorophenyl)amino)isoquinolin-7-yl)-4-(piperidin-1-yl)butanamide

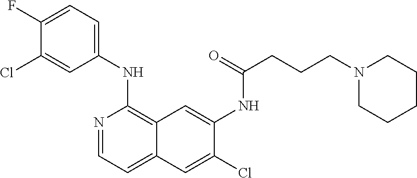 [SMILES: O=C(CCCN1CCCCC1)Nc1cc2c(Nc3ccc(F)c(Cl)c3)nccc2cc1Cl]

To a solution of 6-chloro-N¹-(3-chloro-4-fluorophenyl)isoquinoline-1,7-diamine (200 mg, 0.621 mmol) in DCM (2 mL), 4-(piperidin-1-yl)butanoic acid (240 mg, 1.401 mmol), DIPEA (1 mL, 8.61 mmol) and 50% T3P in EtOAc (0.8 mL, 2.79 mmol) were added at RT and the mixture was stirred at 60° C. for 16 h. The reaction was quenched with addition of MeOH and the mixture was concentrated under vacuum, purified using silica eluting with 10% MeOH in CH₂Cl₂. The final compound was triturated with ether to give the title compound (85 mg, 78.2%) as light brown solid. ¹H NMR (400 MHz, Methanol-d₄) δ 8.62 (s, 1H), 7.97-7.91 (m, 3H), 7.59-7.56 (m, 1H), 7.23-7.14 (m, 3H), 4.85 (s, 2H), 3.66 (d, J=15.1 Hz, 1H), 2.61-2.36 (m, 8H), 2.16 (s, 1H), 2.05-2.01 (m, 2H), 1.68 (s, 4H), 1.53 (s, 1H), 1.33 (d, J=19.2 Hz, 2H). LCMS [M+H]⁺=475.39.

### Example 27: Preparation of N-(1-((3,4-dichlorophenyl)amino)-6-methoxyisoquinolin-7-yl)-4-(piperidin-1-yl)butanamide (I-27)

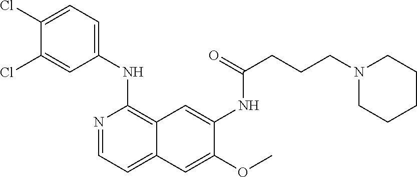 [SMILES: COc1cc2ccnc(Nc3ccc(Cl)c(Cl)c3)c2cc1NC(=O)CCCN1CCCCC1]

To a solution of N-(1-chloro-6-methoxyisoquinolin-7-yl)-4-(piperidin-1-yl)butanamide (150 mg, 0.414 mmol) in dioxane (2 mL) Cs₂CO₃ (269 mg, 0.829 mmol), 3,4-dichloroaniline (73 mg, 0.455 mmol) were added and mixture was degassed with N₂ for 10 min. To this, Xantphos (23 mg, 0.414 mmol) and Pd₂(dba)₃ (37 mg, 0.414 mmol) were added and mixture was stirred at 100° C. for 2 h. After completion of the reaction, mixture was concentrated and the residue was purified by silica gel chromatography eluting with 15-20% MeOH/DCM to afford the title compound (35 mg, 17.3%) as brown solid. ¹H NMR (400 MHz, MeOD-d₄): δ 8.81 (s, 1H), 7.90-7.92 (m, 2H), 7.55-7.53 (m, 1H), 7.43-7.40 (d, J=8.8 Hz, 1H), 7.30 (s, 1H), 7.19-7.18 (d, J=6 Hz, 1H), 4.08 (s, 3H), 2.93-2.86 (m, 5H), 2.67-2.64 (t, J=16.4 Hz, 2H), 1.80-1.74 (m, 4H), 1.51 (s, 2H). LCMS: (M+1)+=487.00.

### Example 28: Preparation of N-(1-((3-ethyl-4-fluorophenyl)amino)-6-methoxyisoquinolin-7-yl)-4-(piperidin-1-yl)butanamide (I-28)

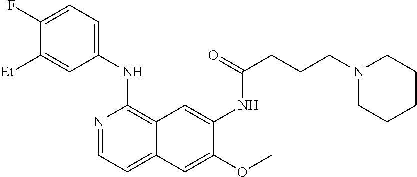 [SMILES: CCc1cc(Nc2nccc3cc(OC)c(NC(=O)CCCN4CCCCC4)cc23)ccc1F]

### Step 1: 3-ethyl-4-fluoroaniline

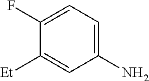

To a suspension of 10% Pd/C (50% moist, w/2, 0.125 g) in EtOAc (4 mL), 3-ethynyl-4-fluoroaniline (0.25 g, 0.184 mmol) was added and the mixture was stirred under hydrogen purging for 3 h. The mixture was filtered through a celite bed and washed with methanol. The organic layer was concentrated under vacuum to afford the product (150 mg, 0.01 mmol) as yellow oil. LCMS: (M+H)⁺=465.2.

### Step 2: N-(1-((3-ethyl-4-fluorophenyl)amino)-6-methoxyisoquinolin-7-yl)-4-(piperidin-1-yl)butanamide

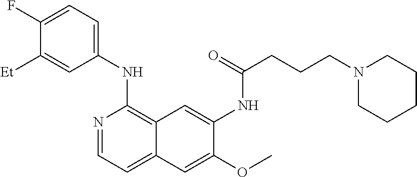 [SMILES: CCc1cc(Nc2nccc3cc(OC)c(NC(=O)CCCN4CCCCC4)cc23)ccc1F]

To a solution of N-(1-chloro-6-methoxyisoquinolin-7-yl)-4-(piperidin-1-yl) butanamide (150 mg, 0.414 mmol) in dioxane (2 mL) Cs₂CO₃ (269 mg, 0.829 mmol), 3-ethyl-4-fluoroaniline (63 mg, 0.455 mmol) were added and mixture was degassed with N₂ for 10 min. Xantphos (23 mg, 0.414 mmol) and Pd₂(dba)₃ (37 mg, 0.414 mmol) were added and mixture was stirred at 100° C. for 2 h. The mixture was concentrated, and the residue was purified by silica gel chromatography eluting with 5-20% MeOH/DCM to afford the title compound (30 mg, 15.6%) as off white solid. ¹H NMR (400 MHz, Methanol-d₄): δ 8.79 (s, 1H), 7.78-7.76 (d, J=6 Hz 1H), 7.45-7.35 (m, 2H), 7.27 (s, 1H), 7.09-7.07 (t, J=6 Hz, 1H), 6.99 (s, 1H), 4.06 (s, 3H), 3.37-3.31 (m, 1H), 2.88-2.83 (t, J=19.2 Hz, 1H), 2.72-2.62 (m, 4H), 2.08-2.03 (m, 2H), 1.77-1.74 (t, J=10.8, 4H), 1.59 (s, 1H), 1.30-1.25 (m, 3H); LCMS: (M+H)⁺=465.2.

### Example 29: Preparation of N-(1-((3-chloro-4-fluorophenyl)amino)-5-methoxyisoquinolin-7-yl)-4-(piperidin-1-yl)butanamide (I-29)

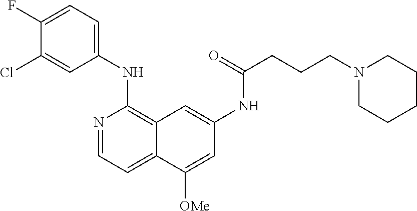 [SMILES: COc1cc(NC(=O)CCCN2CCCCC2)cc2c(Nc3ccc(F)c(Cl)c3)nccc12]

### Step 1: 3-methoxy-5-nitro-N-(pivaloyloxy)benzamide

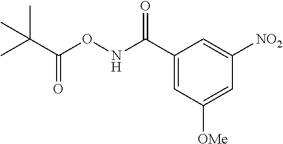 [SMILES: COc1cc(C(=O)NOC(=O)C(C)(C)C)cc([N+](=O)[O-])c1]

To a cooled solution of 3-methoxy-5-nitrobenzoic acid (2.1 g, 10.65 mmol) in DCM (20 mL), oxalyl chloride (1.2 mL, 8.51 mmol) and DMF (catalytic) were added at 0° C. and the mixture was stirred at room temperature for 3 h. The mixture was concentrated under N₂ atmosphere to afford 3-methoxy-5-nitrobenzoyl chloride. To a cooled solution of O-pivaloylhydroxylammonium trifluoromethanesulfonate (1.2 g, 10.08 mmol) and NaHCO₃ (1.7 g, 20.23 mmol) in EtOAc (10 mL) and water (10 mL), the 3-methoxy-5-nitrobenzoyl chloride solution in EtOAc (5 mL) was added at 0° C. and the mixture was stirred at room temperature for 4 h. The mixture was diluted with water (100 mL) and extracted in EtOAc (3×150 mL). The combined organic layers were dried over anhydrous Na₂SO₄ and concentrated under vacuum to afford the title compound (1.4 g, 46.6%) as an off-white solid. The crude material was taken forward to the next step. LCMS: (M+H)⁺=296.2.

### Step 2: 5-methoxy-7-nitroisoquinolin-1(2H)-one

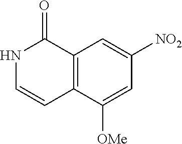 [SMILES: COc1cc([N+](=O)[O-])cc2c(=O)[nH]ccc12]

To a stirred solution of 3-methoxy-5-nitro-N-(pivaloyloxy)benzamide (1.0 g, 3.375 mmol) in MeOH (10 ml), was added vinyl acetate (0.47 g, 5.549 mmol), cesium acetate (0.26 g, 1.352 mmol) and pentamethylcyclopentadienyl rhodium dichloride dimer (40 mg, 0.060 mmol) under nitrogen. After addition, the mixture was stirred at 70° C. for 16 h. After completion of reaction, the mixture was concentrated under vacuum to afford the crude product which was triturated with 1% MeOH in diethyl ether. The solid was filtered and dried over Na₂SO₄ to afford the title compound (0.72 g, 97.0%) as brown solid. LCMS: (M+H)⁺=221.4.

### Step 3: 1-chloro-5-methoxy-7-nitroisoquinoline

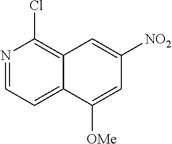 [SMILES: COc1cc([N+](=O)[O-])cc2c(Cl)nccc12]

A suspension of 5-methoxy-7-nitroisoquinolin-1(2H)-one (0.7 g, 3.179 mmol) in phosphorous oxychloride (14 mL) was stirred at 100° C. for 1 h. After completion of the reaction the mixture was concentrated. The residue was diluted with DCM (100 mL) and ice cooled water (100 mL), neutralized with solid NaHCO₃. The layers were separated, and the organic layer was dried over Na₂SO₄, concentrated and triturated with diethyl ether to give (0.68 g, 89.6%) as pale-yellow solid. LCMS: (M+H)⁺=239.2

### Step 4: 1-chloro-5-methoxyisoquinolin-7-amine

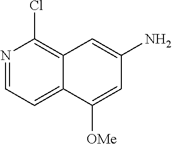 [SMILES: COc1cc(N)cc2c(Cl)nccc12]

To a solution of 1-chloro-5-methoxy-7-nitroisoquinoline (0.9 g, 2.588 mmol) in MeOH (34 mL), water (23 mL) and THF (13 mL) at 60° C., ammonium chloride (0.5 g, 9.301 mmol) and iron powder (0.52 g, 9.325 mmol) were added and the mixture was stirred at 60° C. for 90 minutes. The mixture was filtered, organic was evaporated and product was extracted with EtOAc (2×100 mL). The combined organic layers were dried over Na₂SO₄, concentrated and triturated with ether to give (0.73 g, 92.8%) as brown solid. LCMS: [M+H]⁺=209.0.

### Step 5: N-(6-chloro-1-((3-chloro-4-fluorophenyl)amino)isoquinolin-7-yl)-4-(piperidin-1-yl)butanamide

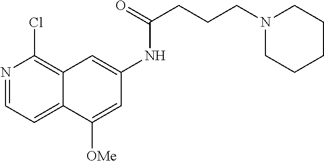 [SMILES: COc1cc(NC(=O)CCCN2CCCCC2)cc2c(Cl)nccc12]

To a solution of 1-chloro-5-methoxyisoquinolin-7-amine (0.3 g, 1.435 mmol) in DCM (3 mL), 4-(piperidin-1-yl)butanoic acid (0.37 g, 2.152 mmol), N,N-diisopropylethylamine (5 mL) and 50% T3P in EtOAc (2.5 mL, 11.5 mmol) were added and the mixture was stirred at 60° C. for 16 h. The mixture was concentrated and purified using silica and eluted in 10% MeOH in MDC to give the title compound (251 mg, 48.4%) as a light brown solid. LCMS: [M+H]⁺=362.

### Step 6: N-(1-((3-chloro-4-fluorophenyl)amino)-5-methoxyisoquinolin-7-yl)-4-(piperidin-1-yl)butanamide

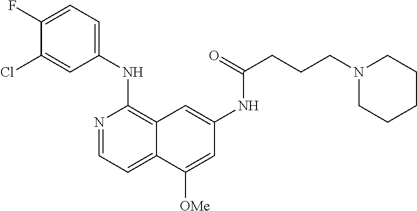 [SMILES: COc1cc(NC(=O)CCCN2CCCCC2)cc2c(Nc3ccc(F)c(Cl)c3)nccc12]

To a degassed solution of 1-chloro-5-methoxyisoquinolin-7-amine (0.15 g, 0.41 mmol), 3-chloro-4-fluoroaniline (90 mg, 0.61 mmol) and Cs₂CO₃ (0.53 g, 1.62 mmol) in 1,4 dioxane (2 mL), tris(dibenzylideneacetone)dipalladium (20 mg, 0.024 mmol) and (9,9-Dimethyl-9H-xanthene-4,5-diyl)bis(diphenylphosphane) (20 mg, 0.034 mmol) were added under N₂ atmosphere and the mixture was stirred at 110° C. for 1 h. The mixture was concentrated and purified using flash chromatography using neutral alumina. The product was eluted in 6% MeOH in DCM and triturated with n-pentane to afford title compound (33 mg, 18%) as a brown solid. ¹H NMR (400 MHz, Methanol-d₄) δ 8.08 (s, 1H), 7.86 (s, 1H), 7.47-7.38 (m, 2H), 7.21-7.14 (m, 2H), 4.01 (s, 3H), 2.56-2.45 (m, 8H), 2.03-1.94 (m, 2H), 1.66-1.64 (m, 5H), 1.32 (d, J=19.2 Hz, 1H); LCMS: [M+H]⁺=471.

### Example 30: Preparation of N-(1-((4-fluoro-3-isopropylphenyl)amino)-6-methoxyisoquinolin-7-yl)-4-(piperidin-1-yl)butanamide (I-30)

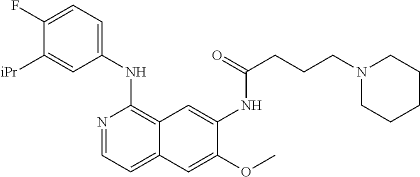 [SMILES: COc1cc2ccnc(Nc3ccc(F)c(C(C)C)c3)c2cc1NC(=O)CCCN1CCCCC1]

### Step 1: 1-fluoro-4-nitro-2-(prop-1-en-2-yl)benzene

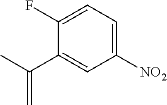

In a flame dried flask, KHMDS (21.6 mL, 20% in THF, 21.85 mmol) was added dropwise under N₂ over 5 min to a stirred suspension of Ph₃P*CH₃Br— (7.6 g, 21.85 mmol) at −78° C. Yellow color appeared as the addition proceeded. After 5 min at −78° C., the suspension was stirred at RT for 5 min and again cooled to −78° C. 1-(2-Fluoro-5-nitrophenyl) ethan-1-one (2 g, 10.9 mmol) was then added dropwise over five minutes. A deep red color appeared as the addition proceeded. The mixture was allowed to warm to RT and concentrated, and the residue was purified by silica gel chromatography eluting with 7% EtOAc in hexane to afford the product (300 mg, 1.66 mmol). ¹H NMR (400 MHz, Chloroform-d): δ 8.26 (d, J=3.2 Hz, 1H), 8.24-8.14 (m, 1H), 7.28-7.16 (m, 2H), 5.40-5.38 (t, J=8 Hz, 2H), 2.19-2.19 (d, J=0.8 Hz, 3H), 1.45 (s, 1H).

### Step 2: 3-ethyl-4-fluoroaniline

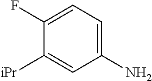 [SMILES: CC(C)c1cc(N)ccc1F]

To a suspension of 10% Pd/C (50% moist, w/2, 0.150 g) in EtOAc (40 mL), 1-fluoro-4-nitro-2-(prop-1-en-2-yl)benzene (0.3 g, 1.65 mmol) was added and the mixture was stirred with hydrogen purging for 3 h. After completion of the reaction, the mixture was filtered through a celite bed and washed with methanol. The organic layer was concentrated under vacuum to afford the product (110 mg, 0.718 mmol) as yellow oil. LCMS: (M+H)+=154.22.

### Step 3: N-(1-((4-fluoro-3-isopropylphenyl)amino)-6-methoxyisoquinolin-7-yl)-4-(piperidin-1-yl)butanamide

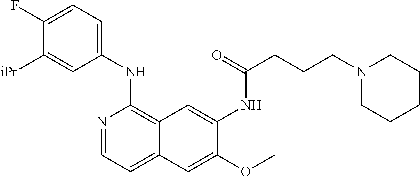 [SMILES: COc1cc2ccnc(Nc3ccc(F)c(C(C)C)c3)c2cc1NC(=O)CCCN1CCCCC1]

To a solution of N-(1-chloro-6-methoxyisoquinolin-7-yl)-4-(piperidin-1-yl) butanamide (100 mg, 0.276 mmol) in dioxane (2 mL) Cs₂CO₃ (270 mg, 0.823 mmol), 4-fluoro-3-isopropylaniline (46 mg, 0.304 mmol) were added and mixture was degassed with N₂ for 10 min. Xantphos (15 mg, 0.276 mmol) and Pd₂(dba)₃ (25 mg, 0.276 mmol) were added and mixture was stirred at 100° C. for 2 h. The mixture was evaporated, the residue was purified by silica gel chromatography eluting with 5-20% MeOH/DCM to provide the title compound (28 mg, 27.2%) as light-yellow solid. ¹H NMR (400 MHz, MeOD-d₄): δ 9.21 (s, 1H), 7.55 (s, 1H), 7.48-7.46 (t, J=9.2 Hz, 1H), 7.42 (s, 1H), 7.31-7.28 (m, 4H), 4.16 (s, 3H) 3.61-3.49 (m, 1H) 3.25-3.23 (m, 4H), 3.01-2.99 (t, J=11.6 Hz, 2H), 2.77-2.73 (t, J=13.6 Hz, 2H) 2.19-2.15 (t, J=16.4 Hz, 2H) 2.00 (s, 1H) 1.96 (s, 1H) 1.85-1.79 (t, J=25.6 Hz, 4H) 1.34-1.30 (t, J=16.8 Hz, 6H). LCMS: (M+H)⁺=479.00.

### Example 31: Preparation of N-(1-((3-chloro-4-fluorophenyl)amino)-6-methoxyisoquinolin-7-yl)-N-methyl-4-(piperidin-1-yl)butanamide (I-31)

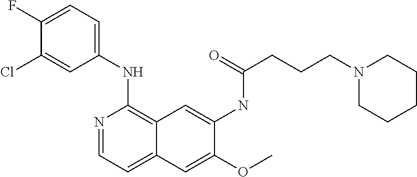 [SMILES: COc1cc2ccnc(Nc3ccc(F)c(Cl)c3)c2cc1NC(=O)CCCN1CCCCC1]

### Step 1: 4-((1-chloro-6-methoxyisoquinolin-7-yl)amino)-4-oxobutanoate

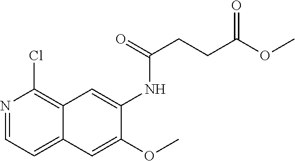 [SMILES: COC(=O)CCC(=O)Nc1cc2c(Cl)nccc2cc1OC]

To a stirred solution of 1-chloro-6-methoxyisoquinolin-7-amine (1 g, 4.79 mmol) and mono-methyl succinate (0.94 g, 7.19 mmol) in DCM (10 V), DIPEA (1.86 g, 14.42 mmol) and T3P (50% in EtOAc) (3.04 g, 9.58 mmol) were added and the mixture was stirred at 70° C. for 16 h. After completion of reaction, the mixture was quenched by water (200 mL) and extracted with EtOAc (3×200 mL) and organic layer was dried over anhydrous Na₂SO₄, concentrated under vacuum to afford the crude product which was purified using column chromatography eluting with 70% EtOAc in hexane to afford the title compound (0.8 g, 51.7%) as brown solid. LCMS: (M+H)⁺=322.75.

### Step 2: methyl 4-((1-chloro-6-methoxyisoquinolin-7-yl)(methyl)amino)-4-oxobutanoate

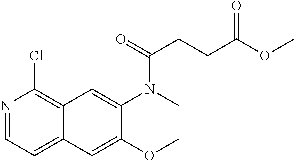 [SMILES: COC(=O)CCC(=O)N(C)c1cc2c(Cl)nccc2cc1OC]

To a stirred solution of methyl 4-((1-chloro-6-methoxyisoquinolin-7-yl)amino)-4-oxobutanoate (0.8 g, 2.48 mmol) and t-BuOK (0.55 g, 4.96 mmol) in DMSO (10 mL), Mel (1.05 g, 7.45 mmol) was added and the reaction mas was stirred at room temperature for 2 h. The mixture was quenched with water (150 mL) and extracted with DCM (2×200 mL). The combined organic layer was dried over anhydrous Na₂SO₄ and concentrated under vacuum to afford the crude product which was purified using column chromatography eluting with 60% EtOAc in Hexane to afford the title compound (0.6 g, 1.78 mmol, 71.9%) as a brown solid. LCMS: (M+H)⁺=336.77.

### Step 3: N-(1-chloro-6-methoxyisoquinolin-7-yl)-4-hydroxy-N-methylbutanamide

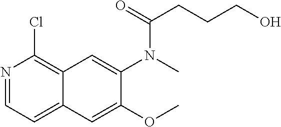 [SMILES: COc1cc2ccnc(Cl)c2cc1N(C)C(=O)CCCO]

To a stirred solution of methyl 4-((1-chloro-6-methoxyisoquinolin-7-yl)(methyl)amino)-4-oxobutanoate (0.6 g, 1.78 mmol) in MeOH (10 V), NaBH₄ (0.66 g, 17.8 mmol) was added at 50° C. The mixture was stirred at 50° C. for 24 h. After completion of reaction, the mixture was concentrated to afford the crude product which was purified using column chromatography eluting with 5% MeOH in DCM to afford the title compound (0.5 g, 71.9%) as light brown solid. LCMS: (M+H)⁺=308.76.

### Step 4: N-(1-chloro-6-methoxyisoquinolin-7-yl)-N-methyl-4-oxobutanamide

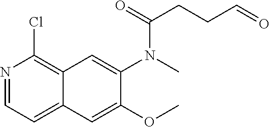 [SMILES: COc1cc2ccnc(Cl)c2cc1N(C)C(=O)CCC=O]

To a stirred solution of N-(1-chloro-6-methoxyisoquinolin-7-yl)-4-hydroxy-N-methylbutanamide (0.5 g, 1.62 mmol) in DMSO (10 V), IBX (0.68 g, 2.43 mmol) was added portion wise, and the mixture was stirred at room temperature for 24 h. After completion of reaction, the mixture was quenched with water (50 mL) and extracted with EtOAc (3×50 mL). The combined organic layers were dried over anhydrous Na₂SO₄ and concentrated under vacuum to afford the crude product which was purified using column chromatography, eluting with 4% MeOH in DCM to afford the title compound (0.4 g, 1.30 mmol, 80.5%). LCMS: (M+H)⁺=306.75.

### Step 5: N-(1-chloro-6-methoxyisoquinolin-7-yl)-N-methyl-4-(piperidin-1-yl)butanamide

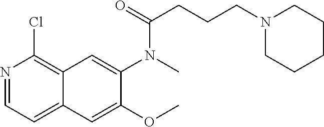 [SMILES: COc1cc2ccnc(Cl)c2cc1N(C)C(=O)CCCN1CCCCC1]

To a stirred solution of N-(1-chloro-6-methoxyisoquinolin-7-yl)-N-methyl-4-oxobutanamide (0.4 g, 1.32 mmol), piperidine (0.22 g, 2.6 mmol) in MeOH (10 V), STAB (0.41 g, 1.95 mmol) was added after 30 min at room temperature and the mixture was stirred at room temperature for 24 h. The mixture was quenched with water (20 mL) and extracted with EtOAc (3×20 mL) and organic layer was dried over anhydrous Na₂SO₄, concentrated under vacuum to afford the crude product which was purified using column chromatography, eluting with 10% MeOH in DCM to afford the title compound (0.25 g, 0.66 mmol, 51%) as an red brown solid. LCMS: (M+H)⁺=375.90.

### Step 6: N-(1-((3-chloro-4-fluorophenyl)amino)-6-methoxyisoquinolin-7-yl)-N-methyl-4-(piperidin-1-yl)butanamide

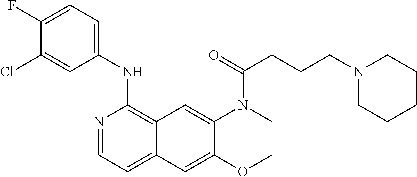 [SMILES: COc1cc2ccnc(Nc3ccc(F)c(Cl)c3)c2cc1N(C)C(=O)CCCN1CCCCC1]

To a degassed solution of N-(1-chloro-6-methoxyisoquinolin-7-yl)-N-methyl-4-(piperidin-1-yl)butanamide (0.15 g, 0.39 mmol), 3-methoxyaniline (63 mg, 0.42 mmol) and Cs₂CO₃ (0.25 g, 0.78 mmol) in dioxane (10 V), to this, Xantphos (22.5 mg, 0.039 mmol) and Pd₂(dba)₃ (35.71 mg, 0.039 mmol) were added under nitrogen atmosphere and mixture was stirred at 100° C. for 1 h. The mixture was quenched with water (10 mL) and extracted with EtOAc (2×20 mL). The combined organic layer was dried over anhydrous Na₂SO₄, concentrated under vacuum to afford the crude product which was purified using column chromatography eluting with 10-12% MeOH in DCM to afford the title compound (40 mg, 21.5%) as light brown solid. ¹H NMR (400 MHz, Methanol-d₄) δ 8.33 (s, 1H), 7.95-7.90 (m, 2H), 7.58-7.54 (m, 1H), 7.40 (s, 1H), 7.3-7.18 (m, 2H), 4.03 (s, 3H), 3.27 (s, 3H), 2.44 (bs, 4H), 2.35-2.30 (s, 2H), 2.24-2.16 (m, 1H), 2.10-2.03 (m, 1H), 1.82-1.74 (m, 2H), 1.58-1.52 (m, 4H), 1.43 (s, 2H); LCMS: (M+H)⁺=485.00.

### Example 32: Preparation of N-(1-((3-chloro-4-methylphenyl)amino)isoquinolin-7-yl)-4-(piperidin-1-yl)butanamide (I-32)

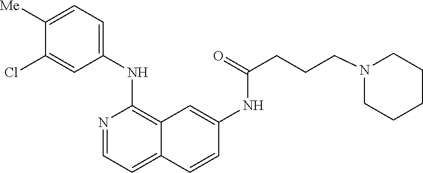 [SMILES: Cc1ccc(Nc2nccc3ccc(NC(=O)CCCN4CCCCC4)cc23)cc1Cl]

To a degassed solution of N-(1-chloroisoquinolin-7-yl)-4-(piperidin-1-yl)butanamide (150 mg, 0.452 mmol) in dioxane (3 mL) Cs₂CO₃ (294 mg, 0.904 mmol), 3-chloro-4-methylaniline (70.4 mg, 0.497 mmol) were added. Xantphos (26 mg, 0.0452 mmol) and Pd₂(dba)₃ (41.38 mg, 0.0452 mmol) were added and the mixture was stirred at 100° C. for 2 h. The mixture was concentrated, and the residue was purified by silica gel chromatography and eluted with 5-20% MeOH/DCM to get (40 mg, 20.3%) as brown solid. ¹H NMR (400 MHz, MeOD-d₄) δ 8.58 (s, 1H), 7.87 (s, 1H), 7.78-7.76 (d, J=8.8 Hz, 2H), 7.67-7.65 (d, J=8.4 Hz, 1H), 7.45-7.43 (d, J=7.2 Hz, 1H), 7.25-7.23 (d, J=8 Hz, 1H), 7.16-7.14 (d, J=4.8 Hz, 1H), 2.56-2.51 (m, 8H), 1.99 (s, 2H), 1.66 (s, 4H), 1.51 (s, 2H). LCMS: (M+H)⁺=436.98.

### Example 33: Preparation of N-(1-((4-fluoro-3-(trifluoromethyl)phenyl)amino)isoquinolin-7-yl)-4-(piperidin-1-yl)butanamide (I-33)

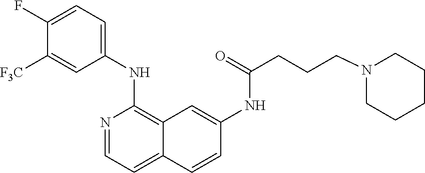 [SMILES: O=C(CCCN1CCCCC1)Nc1ccc2ccnc(Nc3ccc(F)c(C(F)(F)F)c3)c2c1]

To a degassed solution of N-(1-chloroisoquinolin-7-yl)-4-(piperidin-1-yl)butanamide (150 mg, 0.452 mmol) in dioxane (3 mL), Cs₂CO₃ (294 mg, 0.904 mmol), 4-fluoro-3-(trifluoromethyl)aniline (89 mg, 0.497 mmol) were added. Xantphos (26 mg, 0.0452 mmol) and Pd₂(dba)₃ (41.38 mg, 0.0452 mmol) were added and the mixture was stirred at 100° C. for 2 h. The mixture was concentrated and the residue was purified by silica gel chromatography and eluted with 12-20% MeOH/DCM to afford the title compound (50 mg, 23.3%) as brown solid. ¹H NMR (400 MHz, MeOD-d4) δ 8.65 (s, 1H), 8.08-8.07 (d, J=3.6 Hz, 1H), 7.97-7.91 (m, 2H), 7.81-7.79 (d, J=8.8 Hz, 1H), 7.67-7.65 (d, J=8.8 Hz, 1H), 7.32-7.27 (t, J=19. Hz, 1H), 7.21-7.19 (d, J=5.6 Hz, 1H), 2.53-2.48 (m, 8H), 2.03-1.92 (m, 2H), 1.67-1.64 (t, J=10.8 Hz, 4H), 1.51 (s, 2H); LCMS: (M+H)⁺=475.

### Example 34: Preparation of N-(1-((3,4-dichloro-2-fluorophenyl)amino)isoquinolin-7-yl)-4-(piperidin-1-yl)butanamide (I-34)

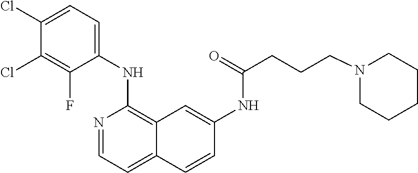 [SMILES: O=C(CCCN1CCCCC1)Nc1ccc2ccnc(Nc3ccc(Cl)c(Cl)c3F)c2c1]

To a degassed solution of N-(1-chloroisoquinolin-7-yl)-4-(piperidin-1-yl)butanamide (150 mg, 0.452 mmol) in dioxane (3 mL) was added Cs₂CO₃ (294 mg, 0.904 mmol), 3,4-dichloro-2-fluoroaniline (89 mg, 0.497 mmol), Xantphos (26 mg, 0.0452 mmol) and Pd₂(dba)₃ (41.38 mg, 0.0452 mmol) and the mixture was stirred at 100° C. for 2 h. The mixture was concentrated and the residue purified by silica gel chromatography eluting with 5-20% MeOH/DCM to afford the title compound (50 mg, 23.3%) as brown solid. ¹H NMR (400 MHz, MeOD-d₄) δ 8.62 (s, 1H), 7.87-7.80 (m, 2H), 7.74-7.68 (m, 2H), 7.38-7.36 (d, J=8.8 Hz, 1H), 7.23-7.23 (d, J=5.2 Hz, 1H), 2.52-2.46 (m, 8H), 2.02-1.94 (m, 2H), 1.66-1.63 (m, 4H), 1.50 (s, 2H). LCMS: (M+H)⁺=475.39.

### Example 35: Preparation of N-(1-((3,4-dichlorophenyl)amino)isoquinolin-7-yl)-4-(piperidin-1-yl)butanamide (I-35)

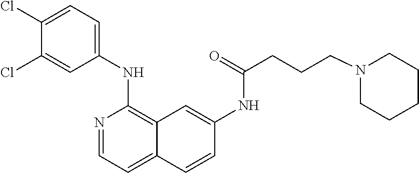 [SMILES: O=C(CCCN1CCCCC1)Nc1ccc2ccnc(Nc3ccc(Cl)c(Cl)c3)c2c1]

To a solution of N-(1-chloroisoquinolin-7-yl-4-(piperidin-1-yl) butanamide (100 mg, 0.276 mmol) in dioxane (2 mL), Cs₂CO₃ (179 mg, 0.552 mmol), 3,4-dichloroaniline (44 mg, 0.304 mmol) were added and reaction vessel was flushed with N₂ for 10 min. To the reaction, Xantphos (15 mg, 0.027 mmol) and Pd₂(dba)₃ (25 mg, 0.276 mmol) were added and mixture was stirred at 100° C. for 2 h. The mixture was concentrated and the residue was purified by silica gel chromatography, eluting with 5-20% MeOH/DCM, to afford the title compound (60 mg, 43.5%) as off white solid. ¹H NMR (400 MHz, MeOD-d₄): δ 8.61 (s, 1H), 8.02 (s, 1H), 7.95-7.94 (d, J=5.2 Hz, 1H), 7.81-7.79 (d, J=9.2 Hz, 1H), 7.67-7.56 (m, 2H), 7.45-7.43 (d, J=8 Hz, 1H), 7.23-7.21 (d, J=5.6 Hz, 1H), 2.52-2.50 (d, J=6.8 Hz, 8H), 2.00 (t, J=3.2 Hz, 2H), 1.65 (s, 4H), 1.45 (s, 2H), 1.30 (s, 1H). LCMS: (M+1)⁺=457.40.

### Example 36: N-(1-((3,5-dichloro-4-fluorophenyl)amino)isoquinolin-7-yl)-4-(piperidin-1-yl)butanamide Preparation of (1-36)

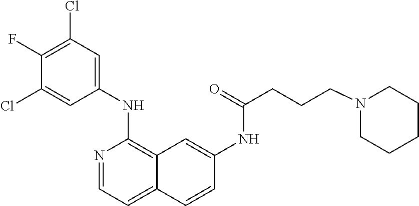 [SMILES: O=C(CCCN1CCCCC1)Nc1ccc2ccnc(Nc3cc(Cl)c(F)c(Cl)c3)c2c1]

To a solution of N-(1-chloroisoquinolin-7-yl)-4-(piperidin-1-yl)butanamide (100 mg, 0.276 mmol) in dioxane (2 mL), Cs₂CO₃ (179 mg, 0.552 mmol) and 3,5-dichloro-4-fluoroaniline (44 mg, 0.304 mmol) were added and reaction mass was degassed with N₂ for 10 min. To this mixture, Xantphos (15 mg, 0.027 mmol) and Pd₂(dba)₃ (25 mg, 0.276 mmol) were added and mixture was stirred at 100° C. for 2 h. The reaction mixture was concentrated, the residue was purified by silica gel chromatography eluting with 5-15% MeOH/DCM to afford the title compound (25 mg, 49.0%) as off white solid. ¹H NMR (400 MHz, MeOD-d₄): δ 8.62 (s, 1H), 7.96-7.95 (d, J=5.6 Hz 1H), 7.87-7.86 (d, J=6 Hz, 2H), 7.81-7.79 (d, J=8.8 Hz, 1H), 7.66-7.64 (d, J=8 Hz, 1H), 7.23-7.22 (d, J=5.6 Hz, 1H), 2.57-2.50 (m, 8H), 2.04-1.98 (m, 2H), 1.68-1.65 (t, J=5.6 Hz, 4H), 1.52 (s, 2H), LCMS: (M+1)⁺=475.39.

### Example 37: Preparation of N-(1-((3,4-dichloro-5-fluorophenyl)amino)isoquinolin-7-yl)-4-(piperidin-1-yl)butanamide (I-37)

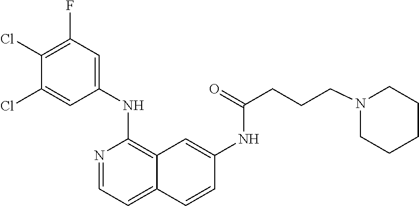 [SMILES: O=C(CCCN1CCCCC1)Nc1ccc2ccnc(Nc3cc(F)c(Cl)c(Cl)c3)c2c1]

To a solution of N-(1-chloroisoquinolin-7-yl)-4-(piperidin-1-yl)butanamide (0.1 g, 0.30 mmol) in 1,4 dioxane (1 mL), 3,4-dichloro-5-fluoroaniline (0.06 g, 0.33 mmol) and Cs₂CO₃ (0.196 g, 0.60 mmol) were added and the reaction mass was flushed with N₂ for 20 min. Tris(dibenzylideneacetone)dipalladium (0.028 g, 0.03 mmol) and (9,9-Dimethyl-9H-xanthene-4,5-diyl)bis(diphenylphosphane) (0.018 g, 0.03 mmol) were added and the reaction mass was stirred at 100° C. for 2 h. The mixture was diluted with water (10 mL) and extracted with EtOAc (3×10 mL). The combined organic layers were dried over anhydrous Na₂SO₄, concentrated under vacuum. The resulting crude product was purified by column chromatography, eluting with 20% MeOH in DCM to afford the title compound (0.07 g, 48.9%) as yellow solid. ¹H NMR (400 MHz, Methanol-d₄) δ 8.63 (s, 1H), 8.00-8.02 (d, J=5.6 Hz, 1H), 7.80-7.82 (d, J=9.6 Hz, 3H), 7.65 (m, 1H), 7.25-7.27 (d, J=5.6 Hz, 1H), 2.63 (m, 6H), 2.55 (t, 2H), 2.01 (t, 2H), 1.67 (m, 4H), 1.54-1.55 (d, J=4.4 Hz, 2H), 0.91 (s, 1H); LCMS [M+1]⁺=475.1.

### Example 38: Preparation of N-(1-((4,5-dichloro-2-fluorophenyl)amino)isoquinolin-7-yl)-4-(piperidin-1-yl)butanamide (I-38)

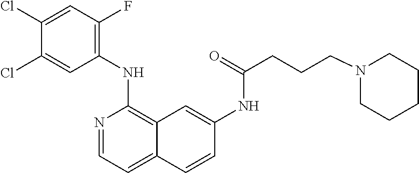 [SMILES: O=C(CCCN1CCCCC1)Nc1ccc2ccnc(Nc3cc(Cl)c(Cl)cc3F)c2c1]

To a solution of N-(1-chloroisoquinolin-7-yl)-4-(piperidin-1-yl) butanamide (100 mg, 0.276 mmol) in dioxane (2 mL), Cs₂CO₃ (179 mg, 0.552 mmol), 4,5-dichloro-2-fluoroaniline (44 mg, 0.304 mmol) were added and reaction mass was degassed with N₂ for 10 min. To this mixture Xantphos (15 mg, 0.027 mmol) and Pd₂(dba)₃ (25 mg, 0.276 mmol) were added and reaction was stirred at 100° C. for 2 h. The mixture was concentrated, and the residue was purified by silica gel chromatography eluting with 10-20% MeOH/DCM to afford the title compound (50 mg, 34.9%) as off white solid. ¹H NMR (400 MHz, MeOD-d₄): δ 8.61 (s, 1H), 8.23-8.20 (d, J=7.2 Hz, 1H), 7.94-7.93 (d, J=1.6 Hz, 1H), 7.84-7.82 (d, J=8.8 Hz, 1H), 7.77-7.75 (d, J=8.4 Hz, 1H), 7.47-7.45 (d, J=10.4 Hz, 1H), 7.26-7.25 (d, J=4 Hz, 1H), 2.81-2.55 (m, 8H), 2.08-2.03 (m, 2H), 1.74-1.73 (d, J=5.2 Hz, 4H), 1.59 (s, 2H). LCMS: (M+1)⁺=475.39.

### Example 39: Preparation of N-(1-((3-chloro-2-fluorophenyl)amino)isoquinolin-7-yl)-4-(piperidin-1-yl)butanamide (I-39)

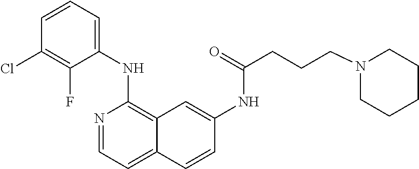 [SMILES: O=C(CCCN1CCCCC1)Nc1ccc2ccnc(Nc3cccc(Cl)c3F)c2c1]

To a solution of N-(1-chloroisoquinolin-7-yl)-4-(piperidin-1-yl)butanamide (100 mg, 0.276 mmol) in dioxane (2 mL), Cs₂CO₃ (179 mg, 0.552 mmol) and 3-chloro-2-fluoroaniline (44 mg, 0.304 mmol) were added and reaction mass was degassed with N₂ for 10 min. To this mixture, Xantphos (15 mg, 0.027 mmol) and Pd₂(dba)₃ (25 mg, 0.276 mmol) were added and reaction was stirred at 100° C. for 2 h. The mixture was concentrated and the residue was purified by silica gel chromatography eluting with 15-20% MeOH/DCM to afford the title compound (45 mg, 33.9%) as off white solid. ¹H NMR (400 MHz, MeOD-d₄): δ 8.62 (s, 1H), 7.86-7.80 (m, 2H), 7.75-7.73 (d, J=8.8 Hz 1H), 7.68-7.67 (d, J=7.2 Hz, 2H), 7.30-7.15 (m, 3H), 2.54-2.49 (m, 8H), 2.03-1.96 (m, 2H), 1.67-1.65 (t, J=10.8 Hz, 4H), 1.51 (s, 2H); LCMS: (M+1)⁺=441.0.

### Example 40: Preparation of N-(1-((3-chloro-2,4-difluorophenyl)amino)isoquinolin-7-yl)-4-(piperidin-1-yl)butanamide (I-40)

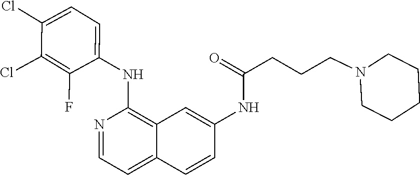 [SMILES: O=C(CCCN1CCCCC1)Nc1ccc2ccnc(Nc3ccc(Cl)c(Cl)c3F)c2c1]

To a solution of N-(1-chloroisoquinolin-7-yl)-4-(piperidin-1-yl) butanamide (100 mg, 0.276 mmol) in dioxane (2 mL), Cs₂CO₃ (179 mg, 0.552 mmol), 3,4-dichloroaniline (44 mg, 0.304 mmol) were added and mixture was degassed with N₂ for 10 min. To this mixture, Xantphos (15 mg, 0.027 mmol) and Pd₂(dba)₃ (25 mg, 0.276 mmol) were added and reaction was stirred at 100° C. for 2 h. The mixture was concentrated and the residue was purified by silica gel chromatography eluting with 5-20% MeOH/DCM to afford the title compound (70 mg, 50.6%) as off white solid. ¹H NMR (400 MHz, MeOD-d₄): δ 8.62 (s, 1H), 7.81-7.79 (d, J=7.2 Hz, 2H), 7.72-7.70 (d, J=8.4 Hz, 1H), 7.58-7.57 (d, J=5.6 Hz, 1H), 7.17-7.13 (t, J=8.8 Hz, 2H), 2.52-2.46 (m, 8H), 2.02-1.94 (m, 2H), 1.66-1.67 (t, J=6 Hz, 4H), 1.50 (s, 2H). LCMS: (M+1)⁺=458.94.

### Example 41: Preparation of N-(1-((3-cyanophenyl)amino)isoquinolin-7-yl)-4-(piperidin-1-yl)butanamide (I-41)

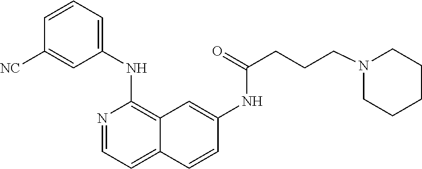 [SMILES: N#Cc1cccc(Nc2nccc3ccc(NC(=O)CCCN4CCCCC4)cc23)c1]

To a solution of N-(1-chloroisoquinolin-7-yl)-4-(piperidin-1-yl) butanamide (100 mg, 0.276 mmol) in dioxane (2 mL), Cs₂CO₃ (179 mg, 0.552 mmol) and 3-aminobenzonitrile (44 mg, 0.304 mmol) were added and reaction flask was flushed with N₂ for 10 min. Xantphos (15 mg, 0.027 mmol) and Pd₂(dba)₃ (25 mg, 0.276 mmol) were added and reaction was stirred at 100° C. for 2 h. The mixture was concentrated and the residue was purified by silica gel chromatography, eluting with 5-20% MeOH/DCM to afford the title compound (60 mg, 48.2%) as off white solid. ¹H NMR (400 MHz, MeOD-d₄): δ 8.65 (s, 1H), 8.20 (s, 1H), 7.98-7.94 (t, J=6 Hz, 2H), 7.83-7.81 (d, J=8.8 Hz, 1H), 7.70-7.67 (d, J=8.8 Hz, 1H), 7.52-7.48 (t, J=7.2 Hz, 1H) 7.36-7.24 (m, 2H), 2.60-2.51 (m, 8H), 2.03-1.92 (m, 2H), 1.70-1.67 (t, J=5.2 Hz, 4H), 1.45 (s, 2H). LCMS: (M+1)⁺=414.52.

### Example 42: Preparation of N-(1-((3-chloro-4-fluorophenyl)amino)-6-methoxyisoquinolin-7-yl)-3-(pyrrolidin-1-yl)propenamide (I-42)

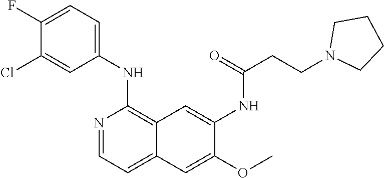 [SMILES: COc1cc2ccnc(Nc3ccc(F)c(Cl)c3)c2cc1NC(=O)CCN1CCCC1]

To a solution of N¹-(3-chloro-4-fluorophenyl)-6-methoxyisoquinoline-1,7-diamine (0.1 g, 0.314 mmol) in THF (5 mL), 3-bromopropanoyl chloride (0.064 g, 0.376 mmol) and DIPEA (0.085 g, 0.628 mmol) were added and the reaction was stirred at 0° C. for 2 h. After 2 h, NaI (0.05 g, 0.314 mmol) and pyrolidine (0.25 g, 3.147 mmol) were added and the mixture was stirred at room temperature for 16 h. The mixture was diluted with brine (20 mL) and extracted with EtOAc (3×10 mL). The combined organic layer was dried over anhydrous Na₂SO₄, and concentrated under vacuum. The resulting crude product was purified using basic alumina eluting with 0.8% MeOH in DCM to afford the title compound (0.035 g, 25.1%) as light brown solid. ¹H NMR (400 MHz, Methanol-d4) δ 8.90 (s, 1H), 7.85-7.81 (m, 2H), 7.52-7.49 (m, 1H), 7.28 (s, 1H), 7.21-7.17 (t, J=18 Hz, 1H), 7.14-7.13 (d, J=6 Hz, 1H), 4.06 (s, 3H), 2.94 (s, 2H), 2.72 (s, 6H), 1.93 (s, 4H). LCMS: (M+H)⁺=442.92.

### Example 43: Preparation of N-(1-((3-chloro-4-fluorophenyl)amino)-6-methoxyisoquinolin-7-yl)-4-(3,3-difluoropyrrolidin-1-yl)butanamide (I-43)

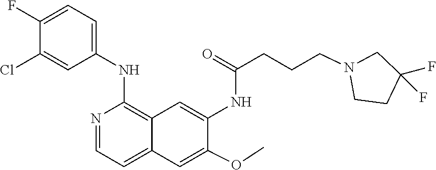 [SMILES: COc1cc2ccnc(Nc3ccc(F)c(Cl)c3)c2cc1NC(=O)CCCN1CCC(F)(F)C1]

### Step 1: methyl 4-((1-((3-chloro-4-fluorophenyl)amino)-6-methoxyisoquinolin-7-yl)amino)-4-oxobutanoate

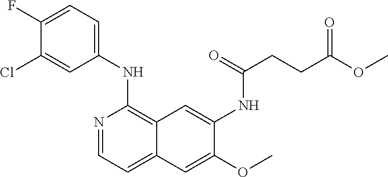 [SMILES: COC(=O)CCC(=O)Nc1cc2c(Nc3ccc(F)c(Cl)c3)nccc2cc1OC]

To a stirred solution of N¹-(3-chloro-4-fluorophenyl)-6-methoxyisoquinoline-1,7-diamine (0.9 g, 2.83 mmol) and mono-methyl succinate (0.56 g, 4.24 mmol) in DCM (5.6 mL), DIPEA (5.6 mL) and T3P (50% in EtOAc, 2.7 g, 8.49 mmol) were added and the reaction was stirred at 70° C. for 16 h. The mixture was quenched with water (100 mL) and extracted with EtOAc (3×100 mL). The combined organic layers were dried over anhydrous Na₂SO₄, concentrated under vacuum to get crude. The crude was purified using column chromatography and the product was eluted in 90% EtOAc in hexane to afford the title compound (0.7 g, 57.2%) as brown solid. LCMS: (M+H)⁺=432.09.

### Step 2: N-(1-((3-chloro-4-fluorophenyl)amino)-6-methoxyisoquinolin-7-yl)-4-hydroxybutanamide

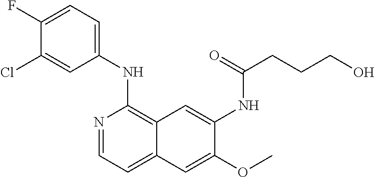 [SMILES: COc1cc2ccnc(Nc3ccc(F)c(Cl)c3)c2cc1NC(=O)CCCO]

To a stirred solution of methyl 4-((1-((3-chloro-4-fluorophenyl)amino)-6-methoxyisoquinolin-7-yl)amino)-4-oxobutanoate (0.7 g, 1.62 mmol) in MeOH (7 mL), NaBH₄ (0.61 g, 16.2 mmol) was added portion wise at 50° C. and the reaction was stirred at 50° C. for 24 h. The mixture was concentrated and the crude product was purified by column chromatography eluting with 5-6% MeOH in DCM to afford the title compound (0.6 g, 91.7%) as an off white solid. LCMS: (M+H)⁺=404.18.

### Step 3: Synthesis of N-(1-((3-chloro-4-fluorophenyl)amino)-6-methoxyisoquinolin-7-yl)-4-oxobutanamide

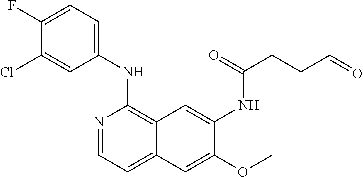 [SMILES: COc1cc2ccnc(Nc3ccc(F)c(Cl)c3)c2cc1NC(=O)CCC=O]

To a stirred solution of N-(1-((3-chloro-4-fluorophenyl)amino)-6-methoxyisoquinolin-7-yl)-4-hydroxybutanamide (0.6 g, 1.49 mmol) in DMSO (6 mL), IBX (0.42 g, 2.98 mmol) was added portion wise and the reaction was stirred at room temperature for 24 h. The mixture was diluted with water (50 mL) and extracted with EtOAc (3×50 mL). The combined organic layers were dried over anhydrous Na₂SO₄ and concentrated under vacuum. The resulting crude product was purified using column chromatography eluting with 4-5% MeOH in DCM to afford the title compound (0.45 g, 75.4%) as light brown solid. LCMS: (M+H)⁺=402.26

### Step 4: N-(1-((3-chloro-4-fluorophenyl)amino)-6-methoxyisoquinolin-7-yl)-4-(3,3-difluoropyrrolidin-1-yl)butanamide

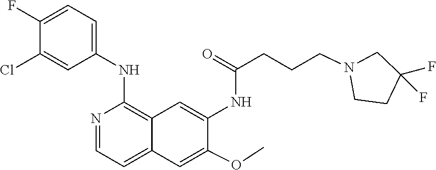 [SMILES: COc1cc2ccnc(Nc3ccc(F)c(Cl)c3)c2cc1NC(=O)CCCN1CCC(F)(F)C1]

To a stirred solution of N-(1-((3-chloro-4-fluorophenyl)amino)-6-methoxyisoquinolin-7-yl)-4-oxobutanamide (0.15 g, 0.37 mmol) and 3,3-difluoropyrrolidine hydrochloride (0.26 g, 1.87 mmol) in MeOH (3 mL), NaCNBH₃ (0.069 g, 1.12 mmol) was added after 30 min at room temperature and the reaction was stirred at 35° C. for 24 h. The mixture was diluted with water (20 mL) and extracted with EtOAc (3×20 mL). The combined organic layer was dried over anhydrous Na₂SO₄ and concentrated under vacuum. The resulting crude product was purified using column chromatography eluting with 8-10% MeOH in DCM to afford the title compound (0.018 g, 9.7%) as an off white solid. ¹H NMR (400 MHz, Methanol-d₄) δ 8.93 (s, 1H), 7.92-7.82 (m, 2H), 7.62-7.53 (m, 1H), 7.39 (s, 1H), 7.36-7.13 (m, 2H), 4.15 (s, 3H), 3.04 (t, J=13.3 Hz, 2H), 2.88 (t, J=7.0 Hz, 3H), 2.42-2.28 (m, 3H), 2.08-1.97 (m, 2H), 1.48 (s, 3H), 1.37 (s, 1H). LCMS: (M+H)⁺=493.

### Example 44: Preparation of N-(1-((3,5-dichloro-2-fluorophenyl)amino)isoquinolin-7-yl)-4-(piperidin-1-yl)butanamide (I-44)

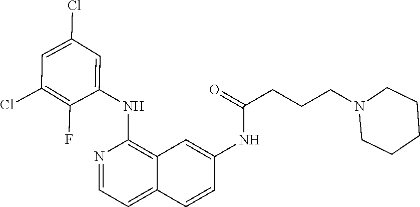 [SMILES: O=C(CCCN1CCCCC1)Nc1ccc2ccnc(Nc3cc(Cl)cc(Cl)c3F)c2c1]

To a degassed solution of N-(1-chloroisoquinolin-7-yl)-4-(piperidin-1-yl)butanamide (0.1 g, 0.30 mmol) in 1,4 dioxane (1 mL), 3,5-dichloro-2-fluoroaniline (0.06 g, 0.33 mmol), Cs₂CO₃ (0.196 g, 0.60 mmol), tris(dibenzylideneacetone)dipalladium (0.028 g, 0.03 mmol) and (9,9-dimethyl-9H-xanthene-4,5-diyl)bis(diphenylphosphane) (0.018 g, 0.03 mmol) were added and the reaction was stirred at 100° C. for 2 h. The mixture was diluted with water (10 mL) and extracted with EtOAc (3×10 mL). The combined organic layers were dried over anhydrous Na₂SO₄, concentrated under vacuum to get the crude. The crude was purified by column chromatography and product was eluted in 20% MeOH in DCM to afford the title compound (0.070 g, 48.9%) as an off white solid. ¹H NMR (400 MHz, Methanol-d₄) δ 8.62 (s, 1H), 7.94 (s, 2H), 7.77 (dd, 2H), 7.23 (m, 2H), 2.71 (m, 5H), 2.58 (m, 2H), 2.02 (m, 2H), 1.73 (m, 4H), 1.57-1.58 (d, J=4 Hz, 2H), 1.28-1.30 (d, J=8 Hz, 1H). LCMS: [M+1]⁺=475.10.

### Example 45. Preparation of N-(1-((4-chloro-2,3-difluorophenyl)amino)isoquinolin-7-yl)-4-(piperidin-1-yl)butanamide (I-45)

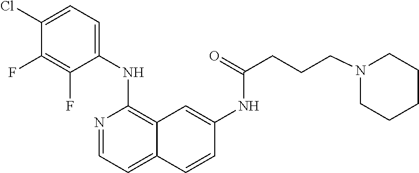 [SMILES: O=C(CCCN1CCCCC1)Nc1ccc2ccnc(Nc3ccc(Cl)c(F)c3F)c2c1]

To a solution of N-(1-chloroisoquinolin-7-yl)-4-(piperidin-1-yl) butanamide (100 mg, 0.276 mmol) in dioxane (2 mL), Cs₂CO₃ (179 mg, 0.552 mmol) and 4-chloro-2,3-difluoroaniline (44 mg, 0.304 mmol) were added and reaction mass was degassed with N₂ for 10 min. To this mixture, Xantphos (15 mg, 0.027 mmol) and Pd₂(dba)₃ (25 mg, 0.276 mmol) were added and reaction mass was stirred at 100° C. for 2 h. After completion of the reaction, the mixture was concentrated and the residue was purified by silica gel chromatography eluting with 10-20% MeOH/DCM to afford the title compound (60 mg, 50.6%) as off white solid. ¹H NMR (400 MHz, MeOD-d₄): δ 8.63 (s, 1H), 7.88-7.82 (m, 2H), 7.75-7.73 (d, J=8 Hz, 1H), 7.53 (s, 1H), 7.30-7.23 (m, 2H), 2.73-2.53 (m, 8H), 2.07-2.01 (m, 2H), 1.73-1.70 (t, J=12 Hz, 4H), 1.56 (s, 2H) 1.34-1.31 (d, J=12 Hz, 1H). LCMS: (M+1)⁺=459.0.

### Example 46. Preparation of N-(1-((3-chloro-4-fluorophenyl)amino)-6-ethoxyisoquinolin-7-yl)-4-(piperidin-1-yl)butanamide (I-46)

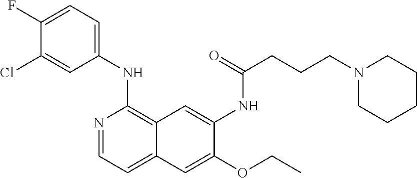 [SMILES: CCOc1cc2ccnc(Nc3ccc(F)c(Cl)c3)c2cc1NC(=O)CCCN1CCCCC1]

### Step 1: 6-fluoro-3,4-dihydroisoquinolin-1(2H)-one

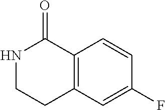 [SMILES: O=C1NCCc2cc(F)ccc21]

To a cooled solution of 5-fluoro-2,3-dihydro-1H-inden-1-one (10 g, 15.02 mmol) in CH₂Cl₂ (100 mL) at 0° C., methane sulfonic acid (43.62 mL, 150.2 mmol) and sodium azide (8.66 g, 30.03 mmol) were added portion wise. The reaction mass was stirred at 0° C. for 2 h. After 2 h, 20% aq. NaOH solution (4 V) was added and the reaction was stirred at RT for 30 min. The mixture was diluted with water (1000 mL) and extracted with CH₂Cl₂ (3×500 mL). The combined organic layers were dried over Na₂SO₄ and concentrated. The resulting crude product was purified using column chromatography eluting with 50% EtOAC:Hexane to afford the title compound (6.5 g, 59.0%) as an off white solid. LCMS: (M+H)⁺=166.0.

### Step 2. 6-fluoro-7-nitro-3,4-dihydroisoquinolin-1(2H)-one

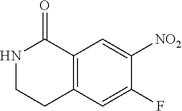 [SMILES: O=C1NCCc2cc(F)c([N+](=O)[O-])cc21]

To a cooled solution of 6-fluoro-3,4-dihydroisoquinolin-1(2H)-one (6.5 g, 39.149 mmol) in sulfuric acid (65 mL) potassium nitrate was added portion wise at 0° C. The reaction mass as stirred at same temperature for 2 h. After completion of reaction, reaction mass was poured in ice water. The resulting precipitate was collected by suction filtration to afford the product (8 g, 96.7%) as a yellow solid. LCMS: (M+H)⁺=211.1.

### Step 3. Synthesis of 6-fluoro-7-nitroisoquinolin-1(2H)-one

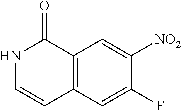 [SMILES: O=c1[nH]ccc2cc(F)c([N+](=O)[O-])cc12]

To a cooled solution of 6-fluoro-7-nitro-3,4-dihydroisoquinolin-1(2H)-one (8 g, 38.06 mmol) in 1,2-dichloroethane (160 mL) at 0° C., manganese(IV) oxide (65.6 g, 761.32 mmol) was added portion wise and the reaction mass was stirred at 100° C. for 48 h. The mixture was concentrated and the resulting crude product was purified by column chromatography eluting with 20% MeOH:CH₂Cl₂ to afford the title compound (6.7 g, 84.6%) as yellow solid. LCMS: (M+H)⁺=209.4.

### Step 4: Synthesis of 1-chloro-6-fluoro-7-nitroisoquinoline

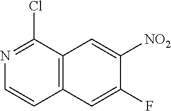 [SMILES: O=[N+]([O-])c1cc2c(Cl)nccc2cc1F]

A suspension of 6-fluoro-7-nitroisoquinolin-1(2H)-one (6.7 g, 32.18 mmol) in phosphoryl chloride (70 mL) was stirred at 100° C. for 16 h. The phosphoryl chloride was removed by distillation and mixture was dissolved in CH₂Cl₂ (500 mL) and quenched by the addition of saturated sodium bicarbonate solution. The organic layer was separated and dried over Na₂SO₄ and concentrated to afford the product (5.5 g, 75.9%) as yellow solid. LCMS: (M+H)⁺=227.06.

### Step 5: N-(3-chloro-4-fluorophenyl)-6-fluoro-7-nitroisoquinolin-1-amine

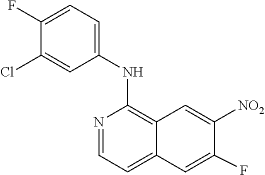 [SMILES: O=[N+]([O-])c1cc2c(Nc3ccc(F)c(Cl)c3)nccc2cc1F]

To a solution of 2-chlororo,3-floroaniline (2.8 g, 19.42 mmol) in 1,4-dioxane (60 mL) at 0° C., 4M hydrochloric acid in dioxane (55 mL) was added slowly and the mixture was stirred at 0° C. for 20 min. To this mixture, 1-chloro-6-fluoro-7-nitroisoquinoline (5.5 g, 24.27 mmol) was added and the mixture was stirred at 60° C. for 16 h. The mixture was diluted with water (100 mL) and extracted with EtOAc (2×100 mL). The organic layer was dried over Na₂SO₄ and concentrated. The resulting crude product was purified using column chromatography eluting with 10% EtOAC:hexane to afford the title compound (4.3 g, 52.7%) as reddish solid. ¹H NMR (400 MHz, DMSO-d₆): δ 9.87 (s, 1H), 9.54 (d, J=8 Hz, 1H), 8.20 (d, J=5.6 Hz, 1H), 8.16 (d, J=4.4 Hz, 1H), 7.99 (d, J=12 Hz, 1H), 7.83 (bs, 1H), 7.42 (t, J=8.8 Hz, 1H), 7.28 (J=7.6 Hz, 1H). LCMS: [M+1]⁺=336.

### Step 6: N-(3-chloro-4-fluorophenyl)-6-ethoxy-7-nitroisoquinolin-1-amine

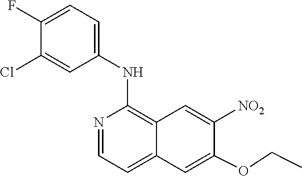 [SMILES: CCOc1cc2ccnc(Nc3ccc(F)c(Cl)c3)c2cc1[N+](=O)[O-]]

To a cooled solution of ethanol (0.122 g, 2.681 mmol) in THF (6 mL) at 0° C., potassium tert-butoxide (0.3 g, 2.68 mmol) was added and the mixture was stirred at 30° C. for 15 min. To this mixture, N-(3-chloro-4-fluorophenyl)-6-fluoro-7-nitroisoquinolin-1-amine (0.6 g, 1.78 mmol) was added and the reaction mass was stirred at 50° C. for 16 h. The mixture was diluted with water (50 mL) and resulting precipitate was collected by filtration and dried to afford the product (0.6 g, 92.9%) as yellow solid. LCMS: (M+H)⁺=362.1.

### Step 7: N1-(3-chloro-4-fluorophenyl)-6-ethoxyisoquinoline-1,7-diamine

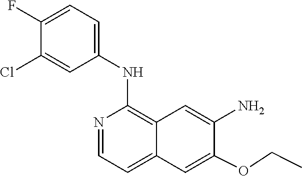 [SMILES: CCOc1cc2ccnc(Nc3ccc(F)c(Cl)c3)c2cc1N]

To a stirred solution of N-(3-chloro-4-fluorophenyl)-6-ethoxy-7-nitroisoquinolin-1-amine (0.6 g, 1.66 mmol) in methanol (20 mL), water (10 mL) and THF (7 mL), iron (0.368 g, 6.64 mmol) and ammonium chloride (0.355 g, 6.64 mmol) were added. The reaction was stirred at 100° C. overnight. The mixture was filtered using a celite bed, and the filtrate was concentrated, diluted with water (50 mL) and extracted with EtOAc (3×30 mL). The organic layer was washed with brine solution, dried over Na₂SO₄ and concentrated to afford the product (0.17 g, 30.9%) as brown color solid. LCMS: [M+H]⁺=332.20.

### Step 8: N-(1-((3-chloro-4-fluorophenyl)amino)-6-ethoxyisoquinolin-7-yl)-4-(piperidin-1-yl)butanamide

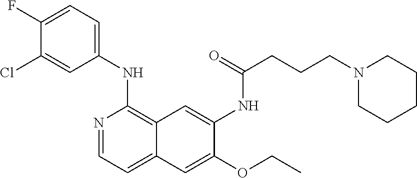 [SMILES: CCOc1cc2ccnc(Nc3ccc(F)c(Cl)c3)c2cc1NC(=O)CCCN1CCCCC1]

To a cooled solution of N1-(3-chloro-4-fluorophenyl)-6-ethoxyisoquinoline-1,7-diamine (0.13 g, 0.392 mmol) in THF (1.2 mL) at 0° C., DIPEA (0.13 mL, 0.78 mmol) and 4-bromobutyryl chloride (0.078 g, 0.47 mmol) were added and the reaction was stirred at 0° C. for 2 h. To this mixture, sodium iodide (0.065 g, 0.393 mmol) and piperidine (0.35 mL, 0.392 mmol) were added and the reaction was stirred at room temperature overnight. The mixture was diluted with water (30 mL) and extracted with EtOAc (3×20 mL). The combined organic layers were dried over Na₂SO₄ and concentrated. The resulting crude product was purified using column chromatography and the product was eluted in 10% MeOH:DCM to afford the title compound (0.093 g, 48.9%) as an off white solid. ¹H NMR (400 MHz, MeOD-d₄): δ 8.77 (s, 1H), 7.85 (d, J=8 Hz, 1H), 7.81 (d, J=4 Hz, 1H), 7.50 (d, J=8 Hz, 1H), 7.27 (s, 1H), 7.19 (t, J=8 Hz, 1H), 7.13 (d, J=8 Hz, 1H), 4.32 (m, J=8 Hz, 2H), 2.99 (m, 5H), 2.68 (t, J=8 Hz, 2H), 2.10 (s, 2H), 1.80 (s, 3H), 1.63 (s, 2H), 1.56 (t, J=4 Hz, 3H), 1.33 (m, 2H), 0.92 (t, J=8 Hz, 1H). LCMS: (M+H)⁺=485.0.

### Example 47: Preparation of (R)—N-(1-((3-chloro-4-fluorophenyl)amino)-6-methoxyisoquinolin-7-yl)-4-(3-methoxypyrrolidin-1-yl)butanamide ((R) 1-47)

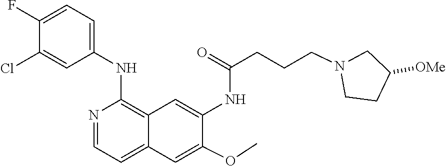 [SMILES: COc1cc2ccnc(Nc3ccc(F)c(Cl)c3)c2cc1NC(=O)CCCN1CC[C@@H](OC)C1]

To a stirred solution of N-(1-((3-chloro-4-fluorophenyl)amino)-6-methoxyisoquinolin-7-yl)-4-oxobutanamide (0.1 g, 0.24 mmol) and (R)-3-methoxypyrrolidine hydrochloride (0.34 g, 2.4 mmol) in MeOH (2 mL), NaCNBH₃ (0.046 g, 0.747 mmol) was added after 30 min at room temperature and the reaction was stirred at 35° C. for 24 h. The mixture was diluted with water (20 mL) and extracted with EtOAc (3×20 mL). The combine organic layers were dried over anhydrous Na₂SO₄ and concentrated under vacuum. The resulting crude product was purified using column chromatography eluting with 8-10% MeOH in DCM to afford the title compound (0.068 g, 56.1%) as light-yellow solid. ¹H NMR (400 MHz, Methanol-d₄) δ 8.80 (s, 1H), 7.82 (dd, J=17.6, 6.6 Hz, 2H), 7.50 (d, J=8.6 Hz, 1H), 7.28 (s, 1H), 7.19 (t, J=9.0 Hz, 2H), 4.06 (s, 5H), 3.22 (d, J=11.9 Hz, 2H), 3.08 (dd, J=11.7, 5.0 Hz, 1H), 2.99 (t, J=8.0 Hz, 3H), 2.66 (t, J=7.0 Hz, 2H), 2.19 (dd, J=14.2, 6.9 Hz, 1H), 2.10-2.02 (m, 4H), 1.30 (s, 1H). LCMS: (M+H)⁺=487.1.

### Example 48: Preparation of (S)—N-(1-((3-chloro-4-fluorophenyl)amino)-6-methoxyisoquinolin-7-yl)-4-(3-methoxypyrrolidin-1-yl)butanamide ((S) 1-47)

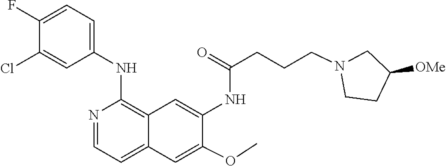 [SMILES: COc1cc2ccnc(Nc3ccc(F)c(Cl)c3)c2cc1NC(=O)CCCN1CC[C@H](OC)C1]

To a stirred solution of N-(1-((3-chloro-4-fluorophenyl)amino)-6-methoxyisoquinolin-7-yl)-4-oxobutanamide (0.1 g, 0.24 mmol) and (S)-3-methoxypyrrolidine hydrochloride (0.34 g, 2.4 mmol) in MeOH (2 mL), NaCNBH₃ (0.046 g, 0.747 mmol) was added after 30 min at room temperature and the reaction was stirred at 35° C. for 24 h. The mixture was diluted with water (20 mL) and extracted with EtOAc (3×20 mL). The combine organic layers were dried over anhydrous Na₂SO₄ and concentrated. The resulting crude product was purified using column chromatography eluting with 5-6% MeOH in DCM to afford the title compound (0.032 g, 26.4%) as light-yellow solid. ¹H NMR (400 MHz, Methanol-d₄) δ 8.79 (s, 1H), 7.84 (s, 2H), 7.50 (m, 1H), 7.28 (s, 1H), 7.24-7.11 (m, 2H), 4.06 (d, J=3.7 Hz, 4H), 2.90 (s, 3H), 2.74 (s, 3H), 2.61 (s, 2H), 2.13 (s, 1H), 2.01 (s, 3H), 1.94 (s, 1H), 1.30 (s, 2H). LCMS: (M+H)⁺=487.2.

### Example 49: (R)—N-(1-((3-chloro-4-fluorophenyl)amino)-6-methoxyisoquinolin-7-yl)-4-(3-fluoropyrrolidin-1-yl)butanamide ((R) 1-48)

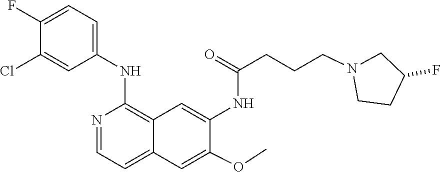 [SMILES: COc1cc2ccnc(Nc3ccc(F)c(Cl)c3)c2cc1NC(=O)CCCN1CC[C@@H](F)C1]

To a stirred solution of N-(1-((3-chloro-4-fluorophenyl)amino)-6-methoxyisoquinolin-7-yl)-4-oxobutanamide (0.1 g, 0.24 mmol) and (R)-3-fluoropyrrolidine hydrochloride (0.31 g, 2.4 mmol) in MeOH (2 mL), NaCNBH₃ (0.046 g, 0.747 mmol) was added after 30 min at room temperature and the reaction was stirred at 35° C. for 24 h. The mixture was diluted with water (20 mL) and extracted with EtOAc (3×20 mL). The combined organic layers were dried over anhydrous Na₂SO₄ and concentrated under vacuum. The resulting crude product was purified using column chromatography eluting with 4% MeOH in DCM to afford the title compound (0.065 g, 55.0%) as light-yellow solid. ¹H NMR (400 MHz, Methanol-d₄) δ 8.79 (s, 1H), 7.87-7.79 (m, 2H), 7.54-7.46 (m, 1H), 7.28 (s, 1H), 7.20 (d, J=9.0 Hz, 1H), 7.16-7.11 (m, 1H), 5.27-5.11 (m, 1H), 4.06 (s, 3H), 3.00 (dt, J=23.6, 12.1 Hz, 2H), 2.62 (dq, J=14.4, 7.7 Hz, 5H), 2.47 (q, J=8.0 Hz, 1H), 2.26-1.94 (m, 4H). LCMS: (M+H)⁺=475.35.

### Example 50: (S)—N-(1-((3-chloro-4-fluorophenyl)amino)-6-methoxyisoquinolin-7-yl)-4-(3-fluoropyrrolidin-1-yl)butanamide ((S) I-48)

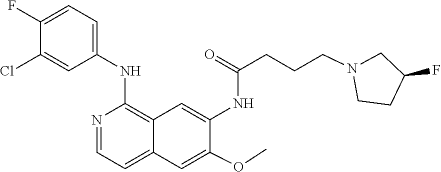 [SMILES: COc1cc2ccnc(Nc3ccc(F)c(Cl)c3)c2cc1NC(=O)CCCN1CC[C@H](F)C1]

To a stirred solution of N-(1-((3-chloro-4-fluorophenyl)amino)-6-methoxyisoquinolin-7-yl)-4-oxobutanamide (0.1 g, 0.24 mmol) and (S)-3-fluoropyrrolidine hydrochloride (0.31 g, 2.4 mmol) in MeOH (2 mL), NaCNBH₃ (0.046 g, 0.747 mmol) was added after 30 min at room temperature and the reaction was stirred at 35° C. for 24 h. The mixture was diluted with water (20 mL) and extracted with EtOAc (3×20 mL), The combine organic layers were dried over anhydrous Na₂SO₄ and concentrated under vacuum. The resulting crude product was purified by column chromatography eluting with 3-4% MeOH in DCM to afford the title compound (0.064 g, 54.2%) as light yellow solid. ¹H NMR (400 MHz, Methanol-d₄) δ 8.79 (s, 1H), 7.84-7.80 (m, 2H), 7.51-7.47 (m, 1H), 7.27 (s, 1H), 7.20-7.12 (m, 2H), 5.28-5.11 (m, 1H), 4.05 (s, 3H), 2.97 (t, J=11.8 Hz, 2H), 2.74 (dd, J=11.9, 5.0 Hz, 1H), 2.63 (dq, J=21.6, 6.5 Hz, 4H), 2.47 (q, J=8.0 Hz, 1H), 2.22 (ddd, J=27.6, 14.0, 7.6 Hz, 1H), 2.13-2.02 (m, 1H), 1.98 (m, 2H), 1.30 (s, 1H). LCMS: (M+H)⁺=475.37.

Using similar methods as described above Compounds 1-49 to 1-53 are prepared.

### B. Biology

### Example 51

i. Method Details

### Protein Purification and Site-Specific Fluorophore Labeling

BAX and BCL-X_{L} were expressed with a C-terminal intein-chitin binding domain used for purification according to the manufacturers recommended procedure (NEB) (Kale et al., 2014; Pogmore et al., 2016). BID and BIM contain an N-terminal histidine tag for nickel column purification (Chi et al., 2020) used both for routine purification and to remove free dye in solution after site-specific labeling of the protein.

Fluorescence-based assays using purified proteins were conducted with single-cysteine mutants to enable site-specific labeling. WT BAX contains two endogenous cysteines (C62 and C126). C62A BAX was used to label C126. Like BAX, BID contains two endogenous cysteines (C30 and C126). Therefore, C30A BID was used to label C126. For compound screens, several batches of BAX were purified and mixed together to obtain more consistent data across a large number of assays.

### Expression of Recombinant Proteins for Purification

For standard protein purification: BAX, BID, BIM and cBAK proteins were expressed in Escherichia coli BL21-Al cells, BCL-X_{L} was expressed in DH5α cells. Proteins were purified as previously described (Moldoveanu et al., 2006; Pogmore et al., 2016; Liu et al., 2019; Bogner et al., 2020). For each purification bacteria were transformed with the requisite expression plasmid and single colonies were picked from agar plates containing 100 μg/mL ampicillin and used to inoculate a 100 mL overnight starter culture of Luria-Bertani (LB) broth supplemented with 100 μg/mL ampicillin. The following day, 50 mL of the overnight culture was used to inoculate two to four 1.5 L flasks of LB supplemented with 100 μg/mL ampicillin. Flasks were then incubated in a shaking incubator at 200 rpm until the OD600 reached 0.7-0.8. 3 g of L-arabinose (for BL21-Al cells) or 1 mM IPTG (final concentration for DH5a cells) were added to induce protein expression. Incubator temperature was then lowered to 20° C. for 24 hours. Bacteria were harvested by centrifugation and the resulting bacterial pellet was stored at −20° C.

For ¹⁵N protein expression: WT BAX was expressed in Escherichia coli BL21-Al cells with slight differences. Instead of picking a single colony, multiple colonies were chosen from ampicillin containing LB-Agar plates and the clone expressing the highest amount of BAX as determined by immunoblotting was stored as a 25% glycerol stock for subsequent high-yield purifications. Overnight cultures (100 mL) were used to inoculate four 1.5 L flasks of minimal media supplemented with 100 μg/mL ampicillin as described (Grant, Marshall and Ikura, 2019). Once the culture reached an OD600 of 0.8, 3 g of arabinose was added to each flask, the incubator temperature was lowered to 20° C., and the cultures were left to incubate for 24 hours. The bacteria were harvested as above, and the resulting bacterial pellet was stored at −20° C. until used for purification.

### Purification of BAX and ¹⁵N-BAX

Standard BAX purification: The bacterial pellet containing full-length BAX fused to a chitin binding domain and a modified self-cleaving intein was thawed at room temperature and resuspended in 50 mL of BAX lysis buffer (10 mM HEPES pH 7, 100 mM NaCl, 0.2% CHAPS, 1 mg DNase, 2 mM PMSF and 2× protease inhibitor cocktail). The resulting solution was drawn through an 18-gauge needle before cell lysis by French press. The lysed bacteria solution was centrifuged at 20 000×g for 30 minutes at 4° C. The pellet was discarded and 1 mL of chitin resin (50% slurry in ethanol) per large 1.5 L flask was added to the supernatant fraction (approximately 45-50 mL total). The supernatant was then incubated at 4° C. in a rotator for 2 hours, then the solution was passed into 15 mL BioRad gravity column to separate the chitin beads from the supernatant. The supernatant was collected and passed over the column twice. The column was then washed with 50 mL BAX wash buffer (10 mM HEPES pH 7, 500 mM NaCl, 0.5% CHAPS). Once the BAX wash buffer has passed through the column, 5 mL of BAX wash buffer supplemented with 300 mM hydroxylamine (pH adjusted back to pH 7 after adding hydroxylamine) was added to the column and allowed to flow through until 1-2 mL of buffer was seen above the chitin bead bed, at which point the column was capped. The column was then incubated for 24 hours at 4° C. to provide time for self-cleavage by the intein to release the protein. Six, 0.5 mL fractions were collected from the column and tested for protein concentration by Bradford assay. Typically, the first three to four fractions that contained the most protein were pooled and thrice passed over a 0.25 mL bed volume of DEAE-Sepharose column equilibrated with BAX wash buffer. The flow through was then dialyzed (12-14 kDa pore size) at 4° C. in 1 L of BAX dialysis buffer (10 mM HEPES pH 7, 200 mM, NaCl, 10% Glycerol). The buffer was replaced twice at 3-4 hour intervals and then dialysis continued in a new 1 L of buffer overnight and collected the next day. The concentration of the purified BAX recovered from the dialysis tubing, was determined by absorbance at 280 nm, and was either used for site-specific labeling with a fluorophore (BAX C62A, 126C) or aliquoted, flash frozen in liquid nitrogen, and stored at −80° C. for future use.

¹⁵N BAX purification: The same protocol seen above was used to purify ¹⁵N BAX from the resulting bacterial pellet with additional steps and the omission of dialysis. The elution fractions from the DEAE column were further purified by gel filtration using a manually packed, G25 fine column equilibrated with BAX phosphate buffer (50 mM potassium phosphate, 50 mM NaCl solution at pH 6) on a BioRAD NGC liquid chromatography system. Fractions containing BAX monomer were pooled and concentrated using a 15 kDa cut-off Centricon spin concentrator (Millipore) and stored at 4° C. for no longer than 24 h prior to NMR experiments. No difference in the activity of BAX was observed with these additional steps but aggregation during NMR was reduced.

Purification of cBAK

The purification of calpain cleaved BAK lacking the C-terminal 24 amino acids (cBAK) was described previously (Moldoveanu et al., 2006). Briefly, residues 1-186 of BAK with an N-terminal FLAG tag and C-terminal calpain recognition site and 6 histidine tag. The bacterial pellet was lysed, centrifuged, and the supernatant containing soluble cBAK was applied to a gravity flow nickel NTA agarose column before further purification using gel filtration chromatography and subsequent anion exchange using HPLC. In a smaller scale to identify the optimal cleaving conditions, the purified protein was subjected to limited proteolysis by calpain in-vitro. Cleaved protein was identified by the appearance of a lower band on SDS-PAGE gel. Samples for SDS-Page were taken at 10-minute intervals to find the optimal time (1 hour) for the calpain reaction to cleave cBAK from the calpain recognition site and histidine tag. The cleaved fractions are then separated by anion exchange. The fractions containing cBAK were pooled, flash-frozen using liquid nitrogen, and stored at −80° C.

Purification of cBID

In cells, BID is activated by cleavage by caspase 8. Therefore, after purification of BID from bacterial lysates it was cleaved by adding His tagged caspase 8. Cleaved BID (cBID) contains two fragments (p7 and p15) which spontaneously dissociate upon interaction with membranes. Truncated BID (tBID also called p15) that activates BAX and BAK remains membrane bound while the p7 fragment is released into solution. Thus, when cBID is added to biochemical reactions tBID accumulates on any added membranes.

To purify cBID, bacterial pellets of cells containing full-length BID were thawed at room temperature and resuspended in 30 mL BID lysis buffer (10 mM HEPES pH 7, 100 mM NaCl, 10 mM imidazole, 1 mM phenyl methyl sulphonyl fluoride (PMSF), and 1 mg bovine DNase I. Cells were lysed using a French press, the lysate was clarified by centrifugation at 20000×g for 30 minutes and the pellet discarded. The supernatant containing BID was applied to a 1 mL bed volume of Nickel NTA agarose beads equilibrated in a gravity flow column with BID lysis buffer. To maximize binding the cleared lysate was passed over the beads 3 times. The beads were then washed with 10 mL BID wash buffer (10 mM HEPES pH 7, 300 mM NaCl, 10 mM imidazole, 1% CHAPS, and 10% glycerol). 1 mL fractions were collected using BID elution buffer (10 mM HEPES, 100 mM NaCl, 0.1% CHAPS, 200 mM imidazole, and 10% glycerol). The eluted protein was detected by spectrophotometry of small aliquots of each fraction after adding Bradford reagent and protein containing fractions were pooled for site specific labeling and/or activation by cleavage. Purified BID or fluorophore labelled BID was incubated in BID cleavage buffer (50 mM HEPES pH 7, 100 mM NaCl, 0.1% CHAPS, 10% glycerol, 1 mM EDTA, and 10 mM DTT) and 500 units of caspase 8 (1 unit/uL cleavage reaction) for approximately two days on a rotator at room temperature. Cleavage efficiency was determined visually after SDS polyacrylamide gel electrophoresis and Coomassie Blue staining. The resulting solution of cBID was dialyzed (12-14 kDa pore size) for 4 hours in 1 L of BID dialysis buffer (10 mM HEPES pH 7, 100 mM NaCl, 0.1 mM EDTA, and 10% glycerol) that was then exchanged and dialysis continued for another 4 hours. The sample was then added to a third liter of dialysis buffer and left overnight. Labeled or unlabeled cBID was then aliquoted, flash frozen with liquid nitrogen, and stored at −80° C.

### Purification of BIM

To purify BIM, bacterial pellet containing full-length BIML was thawed at room temperature and resuspended in 30 mL BIM lysis buffer (20 mM Tris-HCl pH 8, 20 mM NaCl, 1% CHAPS, 20% Glycerol, 5 mM Imidazole, 0.5 mM DTT, 1 mM PMSF, and 1 mg bovine DNase I. Cells were lysed using a French press, the lysate was centrifuged at 20 000×g for 30 minutes, the pellet discarded, and the supernatant containing BIM was mixed with 2 mL of nickel NTA agarose beads (50% slurry in ethanol) and left to incubate on a rotator at 4° C. for 2 hours. The solution was then poured into a 10 mL BioRad gravity flow column and the flow through was passed over the beads twice. 50 mL of BIM wash buffer (20 mM Tris pH 8, 50 mM NaCl, 0.5% CHAPS, 20% glycerol, and 10 mM imidazole) was then applied to the column. BIM elution buffer (20 mM HEPES pH 7, 10 mM NaCl, 0.3% CHAPS, 20% glycerol, and 300 mM imidazole) was used to elute 10 mL from the column. The eluate was then applied to a smaller column with a 400 uL bed volume of high-performance phenyl Sepharose (HPPS) beads. The beads were then washed with 5 mL BIM HPPS wash buffer (20 mM HEPES pH 7, 100 mM NaCl, 0.3% CHAPS, and 20% glycerol). Three 1 mL elution fractions containing BIM were collected using BIM HPPS elution buffer (20 mM HEPES pH 7, 0.3% CHAPS, and 20% glycerol. The presence of eluted protein was detected by Bradford reagent and BIM containing fractions were pooled. To remove CHAPS the pooled fractions were dialyzed (12-14 kDa pore size) in 1 L volumes against a total of 3 L of BIM dialysis buffer (20 mM HEPES pH7, 1 mM NaCl, 20% glycerol). Similar to other proteins, each dialysis step used 1 liter and refreshed after 4 hours, with the third liter left overnight at 4° C. The final sample containing BIM was removed from dialysis tubing, aliquoted, flash frozen with liquid nitrogen, and stored at −80° C.

### Purification of BCL-X_{L}

To purify BCL-X_{L}, the bacterial pellet containing full-length BCL-X_{L} was thawed at room temperature and resuspended in 50 mL of BCL-X_{L} lysis buffer (20 mM Tris-HCl pH 8, 500 mM NaCl, 1% CHAPS, 0.5 mM EDTA, 1 mg bovine DNase I, and 2 mM PMSF). The resulting solution was drawn through an 18-gauge needle before cell lysis using a French press. The lysed bacteria solution was centrifuged at 20 000×g for 30 minutes at 4° C. The pellet was discarded and 1 mL of chitin resin (50% slurry in ethanol) per large 1.5 L flask was added to the supernatant fraction (approximately 45-50 mL total supernatant). The supernatant was then incubated at 4° C. in a rotator for 2 hours, then the solution was poured into a 10 mL BioRad gravity column. The flow through was collected and passed over the column twice. The column was then washed with 50 mL BCL-X_{L} wash buffer (20 mM Tris-HCl pH 8, 200 mM NaCl, 0.2% CHAPS, 20% glycerol, and 1 mM PMSF). Once the wash buffer has passed through the column, 5 mL of BCL-X_{L} wash buffer supplemented with 300 mM hydroxylamine (pH adjusted back to pH 8 after adding hydroxylamine with concentrated NaOH) was added to the column and capped when 1-2 mL of buffer can be seen above the chitin bead bed. The column was then incubated for 24 hours at 4° C. so the intein-chitin binding domain is cleaved from the protein. Six, 0.5 mL fractions were collected from the column and tested for concentration by Bradford assay. Typically, the first three to four fractions contain the most protein, and are pooled, passed over a 0.3 mL bed volume of high-performance phenyl Sepharose (HPPS) beads in a small column. The column was then washed with 5 mL of BCL-X_{L} wash buffer. BCL-X_{L} was eluted from the HPPS beads using BCL-XL elution buffer (Tris-HCl pH 8, 0.2% CHAPS, and 20% glycerol) by collecting three 1 mL fractions. Fractions containing the most protein (determined by Bradford reagent) were pooled for dialysis. The pooled fractions were then dialyzed at 4° C. in 1 L of BCL-X_{L} dialysis buffer (Tris-HCl pH 8, and 20% glycerol), refreshing the buffer every 3-4 hours, twice, and leaving the final liter over night to be collected the next day. The purified BCL-X_{L} is then taken from the dialysis tubing, the concentration of BCL-X_{L} determined by absorbance at 280 nm, and immediately used for fluorescent site-specific labeling or aliquoted, flash frozen in liquid nitrogen, and stored at −80° C. for future use.

### Protein Site-Specific Labeling

BAX and cBID were labeled using a 15× molar excess of Alexa647-C2-Maleimide and Alexa568-C5-Maleimide dissolved in DMSO, respectively, using single cysteine mutants noted above. Proteins were labeled in their respective dialysis buffers supplemented with 0.5% CHAPS for 4-5 hours, rotating at room temperature. The concentration of DMSO should not exceed 10% as the efficiency of labeling will be affected. To remove excess dye from the labeled protein, BID can be re-applied to the nickel NTA agarose column, undergoing the same steps as noted above, while BAX can be passed over a G-25 fine desalting column and eluted using BAX dialysis buffer. Given the Alexa dyes are intense in color, the labeled protein can be seen on the beads which aids in the collection of labeled, protein-containing fractions. The efficiency of the labelling reaction was measured by dividing the concentration of the fluorophore (measured by absorbance at the absorbance maximum for the dye) by the concentration of the protein measured by the absorbance at 280 nm. Labeled proteins were only used if the labelling efficiency was at least 80%.

### Liposome Preparation

The preparation of liposomes and dye-release assays were as previously described (Kale et al., 2014). The composition of liposomes, in molar %, to make a 1 mg/mL lipid film are as follows: 48% phosphatidylcholine, 28% phosphatidylethanolamine, 10% phosphatidylinositol, 10% dioleoyl phosphatidylserine, and 4% tetraoleoyl cardiolipin. Each lipid (dissolved in chloroform) was mixed in a glass test tube. Using a steady stream of nitrogen pointed inside the test tube, the tube is rotated to create a film of lipids on the glass. The tube is filled with nitrogen and covered with parafilm. The dried lipid film is then stored at −20° C. until needed. The lipid film is rehydrated with 1 mL of assay buffer (10 mM HEPES pH 7.2, 200 mM KCl, and 5 mM MgCl₂) and vortexed for 10 seconds. The solution is frozen and thawed 10 times by swirling the test tube in liquid nitrogen, then warm water. The solution of resuspended lipids was then made into a uniform solution of liposomes by extrusion through a 100 nM pore size membrane 11 times.

For ANTS/DPX encapsulated liposomes: 5.3 mg of ANTS and 18.1 mg of DPX were added to the dry lipid film in addition to 1 mL of assay buffer, and then processed as described above.

For Tb:DPA encapsulated liposomes: 1 mL of assay buffer supplemented with 2 mM Tb (III) chloride and 6 mM DPA was added to a dry lipid film, and then processed as described above.

### Liposome Permeabilization and Dye Release Assays

The choice of ANTS/DPX or Terbium:DPA liposomes for dye release assays was dictated by the properties of those compounds being assayed for BAX inhibitory activity, that affected the fluorescence of ANTS or Terbium. Compounds that had fluorescence that overlapped that of either of the encapsulated dyes or that changed the fluorescence of dye-encapsulated liposomes in the absence of added proteins were assayed with the other fluorophore to ensure that the compounds did not simply partition into, or permeabilize the liposomes.

For ANTS/DPX dye release assays: ANTS (fluorophore; excitation 355 nm, emission 520 nm) and DPX (collisional quencher) are encapsulated in reconstituted liposomes. When the membrane is permeabilized, ANTS and DPX diffuse to the outside of the liposomes, decreasing the effective concentration DPX and therefore quenching by ANTS is decreased and ANTS fluorescence increases. ANTS/DPX liposomes (8 uL of mg/mL liposome preparation per 100 μL of reaction) were added to individual wells of multi-well plates (100 μL final volume for 96 well plates or (30 μL total volume for 384 well plates) in assay buffer (10 mM HEPES pH 7.2, 200 mM KCl, and 5 mM MgCl₂). The background fluorescence of the liposomes and the compound dissolved in DMSO was measured for 10 minutes to ensure fluorescent signal stabilized (F₀). Unless explicitly stated, 20 nM cBID and 100 nM BAX were added sequentially to each well and mixed thoroughly. The fluorescence intensity of ANTS was measured every minute for 2-3 hours until the signal stabilizes at a plateau (F). Finally, 2 μL of 10% Triton X-100 was added to each well of a 96 well plate (1 μL/well for 384 well plates) and mixed, taking care not to form bubbles. The plate was then read a third time to assess the fluorescence of ANTS upon full dissociation of liposomes in each well (F₁₀₀). The percentage of dye release from the liposomes due to the action of cBID and BAX is calculated as:

$$\% Dye Release = ( \frac{F - {F}_{0}}{{F}_{100} - {F}_{0}} ) * 100$$

For Terbium:DPA dye release assays: The association of a Tb:DPA results in a fluorescent complex (excitation 280 nm, emission 490 nm) when encapsulated in liposomes. When the liposomes are permeabilized, Tb:DPA diffuse into solution where EDTA chelates the Tb, decreasing fluorescence. Tb:DPA liposomes (8 uL of 0.5 mg/mL lipid preparation per 100 uL reaction) were added to each well of a 96 well (100 μL final volume) or 384 well (30 μL total volume) containing EDTA assay buffer (10 mM HEPES pH 7.2, 200 mM KCl, 5 mM MgCl₂, and 5 mM EDTA). The background fluorescence of the liposomes in the presence of compound (if used) dissolved in DMSO was measured for 10 minutes until the signal remains stable (F₀). Unless explicitly stated, 20 nM cBID and 100 nM BAX were added sequentially to each well and mixed thoroughly. The fluorescence intensity of ANTS was measured kinetically every minute for 2-3 hours until the signal stabilizes at a plateau (F). Finally, 5 μL of 20% CHAPS was added to each well of a 96 well plate or 2 μL for 384 well plate and mixed, taking care not to form an excess of bubbles. The plate was then read a third time to assess the fluorescence of Tb:DPA upon full dissociation of liposomes in each well (F₁₀₀). The percentage of dye release from the liposomes due to the action of cBID and BAX is calculated as:

$$\% Dye Release = ( 1 - ( \frac{F - {F}_{100}}{{F}_{0} - {F}_{100}} ) ) * 100$$

### Measuring BAX Binding to Liposomes and Mitochondria

Compounds were incubated with membranes for 10 minutes at 37° C. before adding purified proteins. Alexa 568, 126C BAX was added and mixed into solution before adding 20 nM BIDmt1 (M97A, D98A; BID mutant that cannot be inhibited by BCL-X_{L}), where specified. All reactions have equivalent DMSO.

For ANTS liposomes, 200 μg/mL lipids in a total reaction volume of 300 uL were incubated at 37° C. for 1 hour before subjecting the mixture to size-exclusion chromatography using a 2.5 mL CL-2B column. Twelve, 200 uL fractions were collected for each reaction and 100 uL were transferred to a 96 well-plate (corning 3881). The fluorescence of ANTS (ex: 355 nm, em: 520 nm) and Alexa 568 (ex: 578 nm, em: 603 nm) were measured on a TCAN M1000, adjusting the gain such that the fluorescence intensity values between the two dyes was similar.

For isolated BAK^{-/-} mitochondria, 1 mg/mL of protein (measured by Bradford assay) in a total reaction volume of 100 uL were incubated at 37° C. for 1 hour before being spun down to pellet the mitochondria. The supernatant was transferred to a different well and the pellet resuspended in 100 μL of assay buffer. The florescence of Alexa 568 BAX was measured in the supernatant and pellet fractions and the percentage of BAX at the membrane was calculated. The nM amount of Alexa 568, 126C BAX was then calculated by multiplying the amount of BAX used in the assay (100 nM or 200 nM, as specified) by the fraction bound to the membrane. To compare BIDmt1 bound BAX with and without BAX inhibitor (dacomitinib or I-1), the fraction of fluorescence intensity of Alexa568-BAX was used to identify the nM amount of BAX bound to the membrane. Each drug condition represents the amount of BAX at the membrane compared to no compound present.

### Mitochondria Isolation and Permeabilization Assays

Animal breeding and handling was performed in accordance with local regulations after approval by the Animal Care Committee at Sunnybrook Research Institute, Toronto. The isolation of mouse-liver mitochondria, and use with Bcl-2 family proteins to measure activity and protein-protein interactions was previously described in detail (Pogmore et al., 2016). In mouse liver, BAX is either cytoplasmic or loosely bound to mitochondria. Therefore, BAX is not present in isolated mitochondria. For this reason, when mitochondria are isolated from liver from BAK−/− mice they contain neither BAX or BAK. In brief, BAK−/− transgenic mice (The Jackson Laboratory, stock number 004183) were sacrificed and the liver tissue collected. 4-5 mouse livers were washed in AT buffer (300 mM Trehalose, 10 mM HEPES-KOH pH 7.7, 10 mM KCl, 1 mM EGTA, 1 mM EDTA, and 0.1% BSA) until erythrocytes were no longer visually apparent. The washed tissue was minced using sharp surgical scissors in 5 mL of ice cold AT buffer per liver used. Minced tissue was homogenized using four strokes of a motor driven Potter-Elvehjem homogenizer. The homogenate was centrifuged for 10 minutes at 600×g, the supernatant collected and centrifuged again for 15 minutes at 3500×g. The supernatant was then collected taking care to avoid the floating adipose layer. A heavy membrane fraction containing mitochondria was obtained by centrifuging at 5500×g for 10 minutes. The pellet was then resuspended in the desired amount of AT buffer, aliquoted for single-use, flash frozen using liquid nitrogen, and stored at −80° C. Prior to use in assays mitochondria are thawed quickly and washed once with AT-KCl buffer (300 mM Trehalose, 10 mM HEPES-KOH pH 7.7, 80 mM KCl, 1 mM EGTA, 1 mM EDTA, and 0.1% BSA) and pelleted by centrifuging the solution at 10 000×g. The supernatant was discarded, and the remaining pellet is resuspended in regeneration buffer (300 mM Trehalose, 10 mM HEPES-KOH pH 7.7, 80 mM KCl, 1 mM EGTA, 1 mM EDTA, 0.1% BSA, 5 mM succinate, 2 mM ATP, 10 μM phosphocreatine, and 10 μg/mL creatine kinase) at a protein concentration of 1 mg/mL, (determined by Bradford assay) for immediate use as the source of membranes for FRET measurements.

Mitochondria from BAX^{-/-}/BAK^{-/-} or BAX^{-/-} baby mouse kidney (BMK) cells stably expressing a fusion protein composed of the import peptide of SMAC and the fluorescent protein, mCherry were isolated and used as previously described (Niu et al., 2017). SMAC-mCherry BMK cells were maintained in DMEM containing 10% FBS supplemented with 3 μg/mL Blasticidin. Cells were scraped from tissue culture dishes, placed on ice, collected with PBS, and centrifuged at 500×g for 4 minutes to pellet the cells. The supernatant was removed and the cell pellet was resuspended in lysis buffer (20 mM HEPES pH 7, 250 mM sucrose, 150 mM KCl, 1 mM EDTA, and 2× protease inhibitor cocktail) and the cells were lysed by nitrogen cavitation at 250 psi for 10 minutes with the containment chamber surrounded by ice. Cell debris were removed by centrifugation at 2000×g for 4 minutes at 4° C. The supernatant was retained and centrifuged at 13 000×g for 10 minutes to pellet heavy membranes containing mitochondria. The pellet was then diluted in lysis buffer to 0.2 mg/ml protein concentration (determined by Bradford assay) for immediate use in permeabilization experiments.

For BAX^{-/-}/BAK^{-/-}BMK SMAC-mCherry mitochondria, 2 nM cBID and 20 nM BAX was used to fully permeabilize the mitochondria. After a 30-minute incubation at 37° C., the 50 μL reactions were centrifuged to pellet the mitochondria to separate the supernatant. The pellet was resuspended in an equal amount of lysis buffer. The percentage of SMAC-mCherry release was determined by fluorometric analysis of the supernatant and pellet fractions by measuring the fluorescence intensity (F) of mCherry (excitation 580, emission 610 in a Tecan M1000).

### Förster Resonance Energy Transfer

A detailed method for the FRET assays with isolated mitochondria was previously described (Pogmore et al., 2016). Briefly, compounds were dissolved in DMSO, incubated with 1 mg/mL mitochondria and 5 nM Alexa568-cBID (FRET donor) at 37° C. before titrating the labeled Alexa647-BAX (FRET acceptor) or unlabeled BAX (to account for donor fluorescence changes due to chemical environment changes due to protein binding). A measurement was taken every minute for 1-2 hours, until the fluorescence intensity of the donor fluorophore reached a plateau. The FRET efficiency (E) was calculated by subtracting the quotient of the fluorescence intensity of the donor labeled protein in the presence of the labeled acceptor protein (F_{DA}) by the fluorescence intensity of the donor fluorophore in the presence of the unlabeled acceptor protein (F_{D}). Since the background for the Alexa dyes in the presence of mitochondria are negligible, the equation to calculate E is as follows:

$$( 1 - ( \frac{{F}_{DA}}{{F}_{D}} ) ) * 100$$

### Kinase Assays

A peptide containing the kinase domain of EGFR (amino acids 695-1210) was used to assess the inhibitory potency of the inhibitors. The ADP-Glo kinase assay (Promega V9101), and the EGFR kinase enzyme (Promega V3831) were used according to the supplier's instructions. Briefly, 26 μL reactions were assembled in wells of a 384 well plate as follows: 5 μL of the kinase reaction, 5 μL of the ADP depletion reagent, and 10 μL of the luciferase reagent. For the kinase reaction: 1 μL of the inhibitor (dissolved in DMSO) was mixed with 2 μL EGFR peptide (0.1 mg/μL), 1 μL of a poly Glu₄Tyr₁ peptide (0.2 μg/μL), 1 μL reaction buffer, and 1 μL of ATP (final concentration of 5 μM). The kinase reaction was incubated at room temperature for 1 hour. 5 μL of the ATP depletion/kinase terminating reagent was mixed into each well and incubated at room temperature for 40 minutes. 10 μL of the kinase detection reagent containing luciferase was mixed into each well and incubated at room temperature for 30 minutes for bioluminescent detection of ADP produced from the kinase reaction in step 1. Bioluminescent signal was detected in a Tecan Safire² with a black plug covering the excitation filter, and an empty emission filter. The integration time was increased to 0.5 μs to detect enough luminescence per reading.

For the kinase panel, % inhibition was measured using standardized assay conditions at the KinaseProfiler service (Eurofins Pharma Discovery Services). Compounds were sent for testing as dry powder.

### Micro-Scale Thermophoresis

MST measurements were taken with a Monolith NT.115 with LED power of 60%, and IR laser time of 25s (final off time 5s), and IR laser power at 50%, and the machine temperature set to 25° C. Each reaction only differed in the concentration of compound. Each reaction contained 100 nM Alexa647 labeled BAX, 0.5% DMSO and phosphate buffer (100 mM KCl and 150 mM NaCl) at pH 7.3. Reaction tubes were sonicated for ˜5s to dissolve the compounds at higher concentrations before being transferred to capillary tubes. No difference in activity was observed with sonicated BAX in liposome dye release experiments similar to other studies (Garner et al., 2019).

### NMR Spectroscopy and Chemical Shift Perturbations

All experiments were performed using an independent sample for each measurement and were DMSO matched to account for environment changes due to the solvent. ¹⁵N-labeled BAX was prepared as described above in 50 mM potassium phosphate pH 6, 50 mM NaCl, and 10% D₂O. Samples were mixed before loading 40 μL in a 1.7 mm sample tube. At the concentrations used here no change in pH was observed from the addition of dacomitinib to samples containing BAX. NMR experiments used full-length WT BAX at a concentration of 200 μM. Experiments were conducted on a 600 MHz Bruker Avance magnet at 25° C. Chemical shift perturbations (CSP) were calculated in CCPNMRv3 using an automated analysis, where all peaks were matched to their extrema and calculated using the equation:

$$\sqrt{{( Δ {δ}_{Hi} )}^{2} + {α ( Δ {δ}_{Ni} )}^{2}}$$

In the CSP equation above, α denotes the relative weighting of chemical shift changes of the ¹⁵N relative to ¹H nuclei. Δδ_{HX} and Δδ_{Nx} are the observed changes of the proton and nitrogen chemical shifts for residue x, respectively. As a note, all amino acid positions on the HSQC were manually inspected to use the extrema of the nearest identified peak. The absence of a bar in the CSP graph indicates no chemical shift, the presence of a proline, or a missing residue peak not used for analysis. Significance threshold was calculated as the average of chemical shifts across all residues plus the standard deviation. For residue significance representation, shifts over the significance threshold are light grey whilst shifts over 1.5 times the significance threshold are dark grey.

### Cell Culture

Baby Mouse Kidney (BMK) cells were from (Mathew et al., 2008). BMK double knockout, SMAC-mCherry, and BAX^{-/-} cell lines were generated in house as described (Niu et al., 2017). BMK lines were grown in DMEM supplemented with 10% Fetal Bovine Serum and 1×MEM non-essential amino acids in a humidified incubator at 37° C. and 5% CO2.

For cell death assays with actinomycin D, 3000 WT or DKO BMK cells were cultured in a 384 well plate and incubated overnight. The next morning, cells were treated with actinomycin D for 2 hours before the addition of BAX inhibitor. Actinomycin D and BAX inhibitor (where relevant) were then incubated for 24 hours before staining with Hoechst, AnnexinV-Alexa488, and TMRE for 30 minutes. Cells were imaged by confocal microscopy (Opera Phenix—PerkinElmer) and subsequently analyzed with Harmony (PerkinElmer) software. Cell death was scored by three criteria: the loss of TMRE signal, the appearance of AnnexinV-Alexa488 signal, and nuclear shrinkage visualized by Hoechst.

ii. Results
Kinase Inhibitor Structures that Inhibit cBID—BAX Mediated Liposome Permeabilization

The 4-anilinoquinazoline-containing compound, dacomitinib was identified in a screen for compounds that inhibit c-BID-activated BAX-mediated liposome permeabilization.

### Compounds Directly Bind to BAX

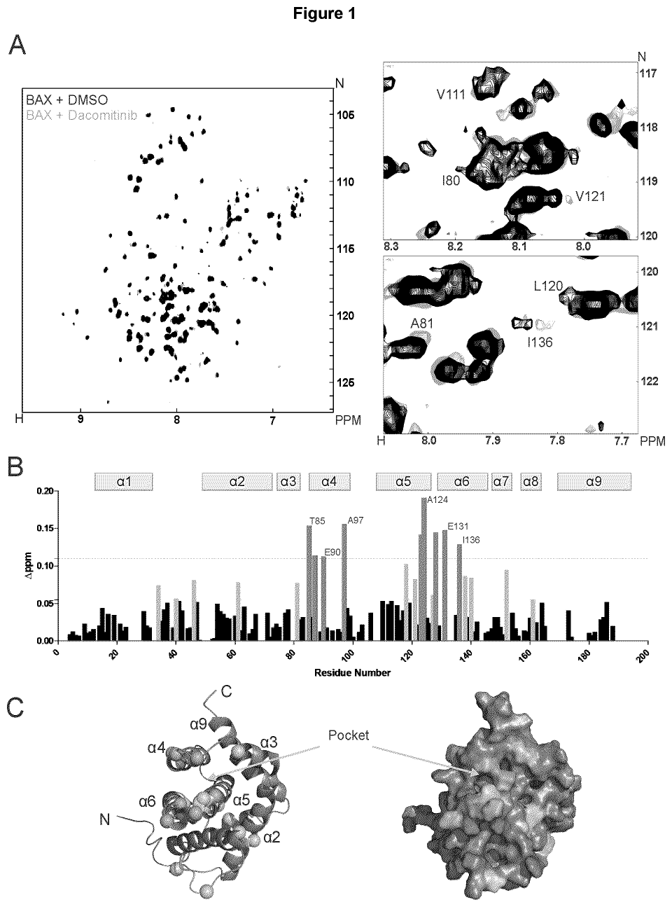

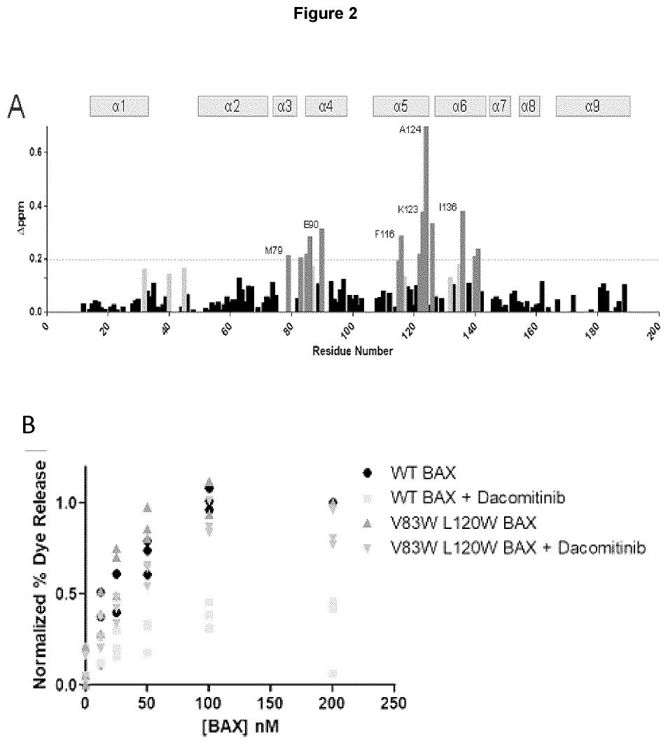

Identifying the binding site of dacomitinib and compound I-1 would ideally be achieved by high resolution 3D structures of BAX and compound. However, such measurements are difficult due to technical challenges including the dynamic structure of BAX and the limited solubility of compounds. Therefore, to identify where dacomitinib and 1-1 bound to BAX, 2D ¹H-¹⁵N heteronuclear single quantum coherence (HSQC) NMR analysis was performed with ¹⁵N-labelled BAX (FIG. 1A). Analysis of the chemical shift perturbations (CSPs) of BAX in the presence of dacomitinib (FIG. 1 B, C) and I-1 (FIG. 2 A) indicates significant chemical shifts localized in the pocket of α-helices 4, 5, and 6. Additional CSPs adjacent to α-helices 4, 5, and 6 are less significant and are likely the result of induced fit conformational changes when dacomitinib is bound to BAX. To further support the proposed location dacomitinib and I-1 binding site, the V83W, L120W mutant of BAX proposed to block the BAl1 binding site between α-helices 4, 5, and 6 (Garner et al., 2019) was used. These mutations in the recombinant protein were observed to also impede inhibition of BAX by dacomitinib (FIG. 2 B). Taken together, these data suggest that dacomitinib as well as I-1 bind the cytosolic conformation of BAX near the previously identified binding site for BAl1 and that binding does not inhibit BH3 peptide binding. The NMR data further suggests that the effects of I-1 binding on the structure of BAX are slightly more pronounced for I-1 consistent with tighter binding.

### Hit Compounds Reduced BAX/BAK Permeabilization of Mitochondrial Outer Membranes

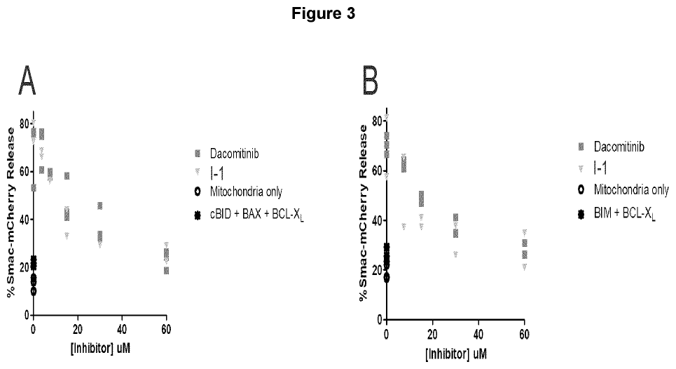

To assess the inhibitory activity of dacomitinib and I-1 on BAX or BAK mediated permeabilization of mitochondria, heavy membranes were isolated from BAX^{-/-} BAK^{-/-} baby mouse kidney (BMK) or BAX^{-/-} BMK cells expressing Smac-mCherry, a fluorescent protein located in the intermembrane space of mitochondria consisting of the import peptide of Smac fused to mCherry. Heavy membrane fractions from these cells were incubated with sufficient recombinant BAX and cBID proteins to release between 60 and 80% of the Smac-mCherry from the intermembrane space of the mitochondria. A concentration dependent reduction in the percentage of Smac-mCherry released was measured in reactions in which the compounds were added at concentrations up to 60 uM (FIG. 3 A). Consistent with the results obtained using liposomes, the inhibition curves were similar for both of the compounds, suggesting they have similar activities. Unexpectedly, much higher compound concentrations were required to inhibit MOMP than were required to inhibit liposome permeabilization. This may be due to non-specific sticking of the compounds to the membrane fractions, degradation of the compounds by microsomal and mitochondrial enzymes present in heavy membrane fractions or poor solubility of the compounds in incubations containing heavy membranes. To assay inhibition of full length BAK, the same range of compound concentrations was added to incubations containing recombinant activator protein BIM and a heavy membrane fraction isolated from BAX^{-/-} mitochondria (FIG. 3 B). As a positive control in both experiments the anti-apoptotic protein, BCL-X_{L}, was used to inhibit the BH3 protein activator and BAX or BAK. Together the data suggest that dacomitinib and I-1 are equally effective inhibitors of BAX and BAK independent of the activator BH3 protein.

### Kinase Binding is Dispensable for BAX Inhibition.

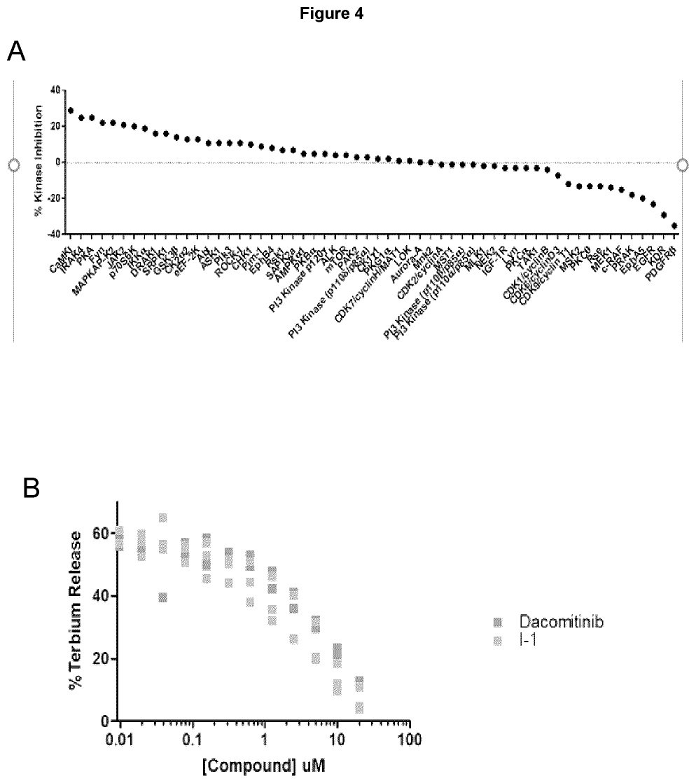

Compound I-1, which is a variant of dacomitinib lacking both the Michael acceptor and the hinge-binding N-1 nitrogen of the quinazoline ring has no appreciable kinase inhibitory activity at or below 10 uM (Table 1). In fact, removal of the hinge-binding nitrogen led to minimal activity across a diverse panel of 58 kinases (FIG. 4A). Nevertheless, unexpectedly, exemplary compound I-1 retained BAX inhibitory activity equivalent to comparative compound dacomitinib when assayed for inhibition of cBID activated, BAX mediated permeabilization of mitochondrial like liposomes (FIG. 4B). In summary, the data for compound I-1 demonstrates clearly that the quinazoline core structure and kinase inhibition of the dacomitinib class of compounds is unnecessary for BAX inhibition. In addition, compound I-1 meets the drug-like predictive requirements for molecules with favorable ADME.

TABLE 1

| | | FL EGFR | Hill |
|---|---|---|---|
| Compound | Structure | IC50 (nM) | Slope |
| Dacomitinib | 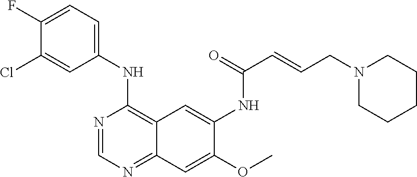 [SMILES: COc1cc2ncnc(Nc3ccc(F)c(Cl)c3)c2cc1NC(=O)/C=C/CN1CCCCC1] | 4 | −1.3 |
| 1-1 | 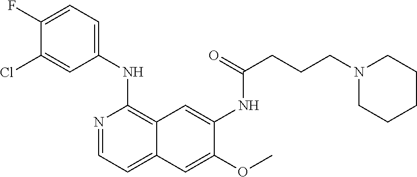 [SMILES: COc1cc2ccnc(Nc3ccc(F)c(Cl)c3)c2cc1NC(=O)CCCN1CCCCC1] | >10,000 | N/D |

BAX Inhibition Rescues Baby Mouse Kidney Cells from Actinomycin D Induced, BAX and BAK Mediated, Cell Death

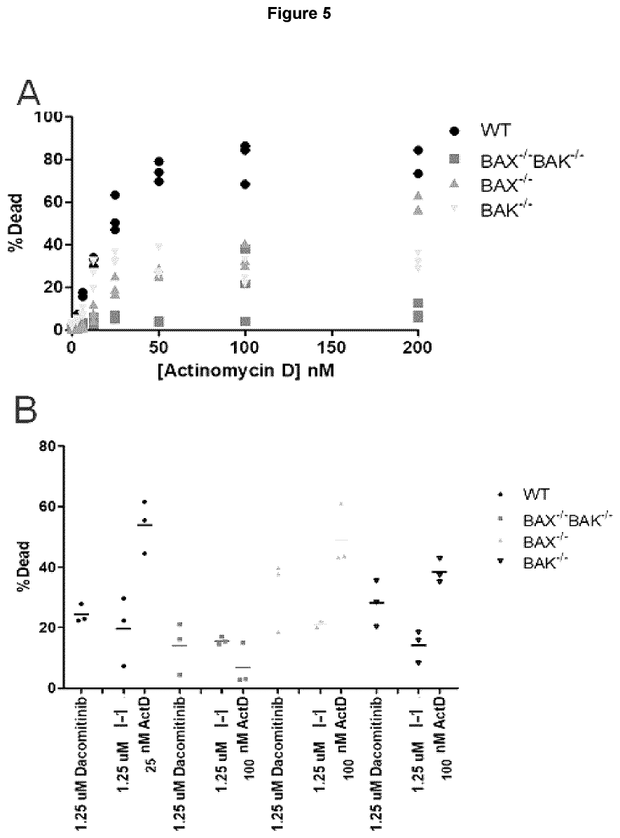

To test the effects of dacomitinib and compound I-1 on cell death should utilize a method to induce cell death in a manner that exclusively depends on BAX and BAK. Actinomycin D is an inducer of BAX and BAK mediated apoptosis (FIG. 5A) and therefore, was used at a concentration that killed ˜80% of wild-type cells but did not kill BAX^{-/-},BAK^{-/-} double knockout BMK cells (FIG. 5 A-B). Single knockouts of BAX or BAK were also used to assess the contribution of the individual executioner proteins to actinomycin D induced apoptosis. At 100 nM, actinomycin D induced cell death equivalently in both single-knockout cell types demonstrating the drug leads to activation of both executioner proteins. Moreover, cell death in the single knockouts was approximately half that of the wild-type suggesting at that concentration of actinomycin D the two executioner proteins contribute equally to the cell death observed. At higher concentrations of the drug, cell death increased further for cells expressing BAX than BAK an effect that may be related to the total protein concentration(s) of the executioner or BH3 proteins in the cells. Based on the data in FIG. 5 A, concentrations of actinomycin D of 25 nM and 100 nM were chosen for the wild-type and the knock-out cell lines, respectively to provide a comparable dynamic range for titration of the inhibitors. Based on data from preliminary experiments, either 1.25 or 2.5 uM concentrations of dacomitinib or 1-1 were used to protect each cell type from the indicated concentration of actinomycin D (FIG. 5 B). Based on these data, compound I-1 was most effective inhibitor of actinomycin D mediated cell death in cells expressing either BAX or BAK. However, comparison of titration data for each of the inhibitors across multiple replicates suggests that overall, the different molecules inhibit BAX and BAK more or less equivalently.

Inhibition of BAX and BAK Protects BMK Cells from Apoptosis Induced by Actinomycin D

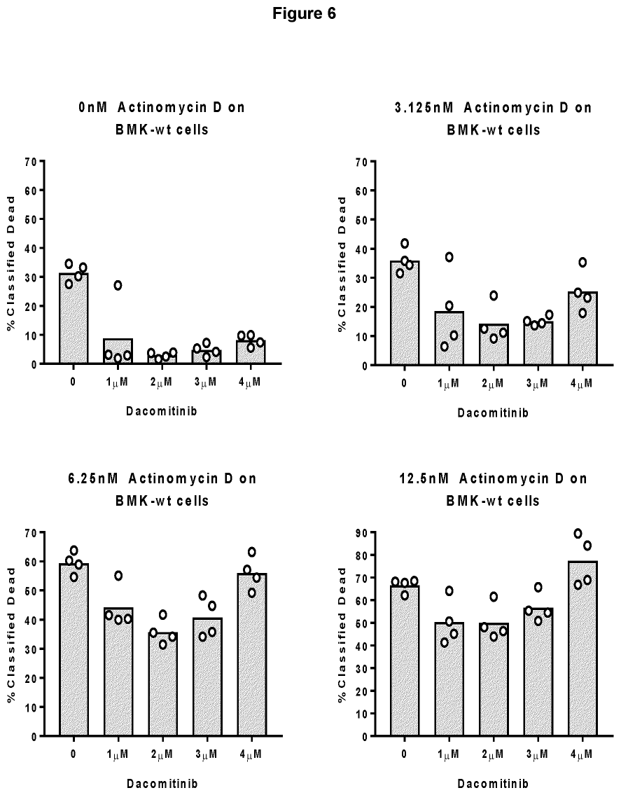

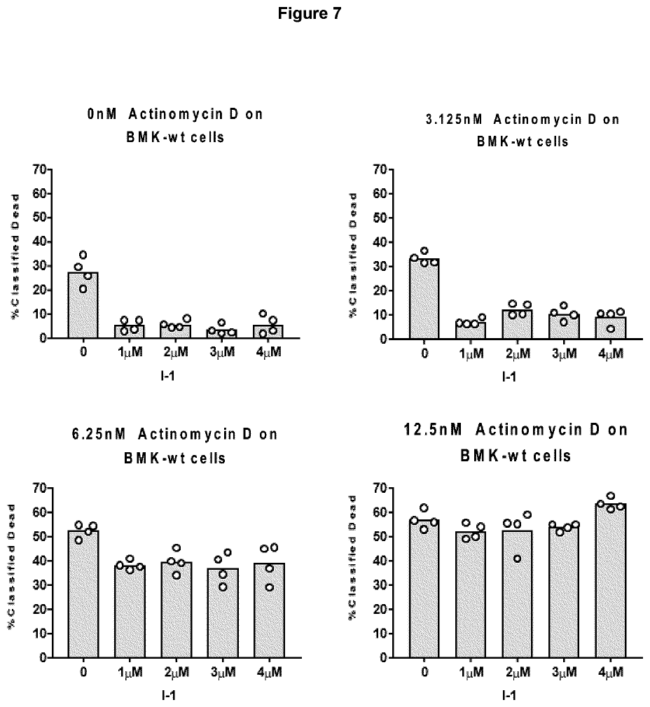

Wild type BMK cells (BMKwt) were treated with different concentrations of actinomycin D and dacomitinib (FIG. 6) or compound I-1 (FIG. 7). Cell death was assessed using a linear classifier based on measurement of mitochondrial transmembrane potential as measured by TMRE (Tetra-Methyl Rhodamine Ester), by nuclear shrinkage visualized with Hoechst and by binding of fluorescent Annexin V. Dacomitinib and I-1 were well-tolerated by the cells and reduced the amount of spontaneous cell death in the cultures (0 mM actinomycin D). Dacomitinib (FIG. 6) and compound I-1 (FIG. 7) reduced cell death induced by concentrations of actinomycin D that do not kill BMK cells lacking both BAX and BAK. Taken together, these data demonstrate that dacomitinib and I-1 inhibit cell death due to actinomycin D mediated activation of BAX and/or BAK.

iii. Discussion

Dacomitinib, an FDA approved kinase inhibitor was surprisingly found to have off-target inhibitory activity for BAX and BAK. To the best of the Applicant's knowledge, dacomitinib is the first FDA approved compound shown to inhibit both BAX and BAK. Dacomitinib is a well-tolerated, orally available small molecule which provided a scaffold for investigation of further pharmaceutically useful inhibitors of BAX and BAK. It was further unexpectedly demonstrated that the kinase inhibitor activity of the dacomitinib is not required for inhibition of BAX and BAK. Consistent with this notion, compound I-1 was identified as an example of a new family of compounds with an isoquinoline core structure that lacks kinase inhibitory activity yet still inhibits both BAX and BAK. While not wishing to be limited by theory, NMR data suggest that dacomitinib and I-1 bind BAX at a solution-available pocket at the junction between a helices 4, 5, and 6 (FIGS. 1-2).

### Example 52: SMAC mCherry Assay

Bax−/− Bak−/− double-knockout Baby Mouse Kidney (BMK) cells stably expressing a SMAC-(1-56)-mCherry fusion protein were cultured in DMEM complete (DMEM supplemented with 10% FBS and 1× Penicillin-Streptomycin) in 10 cm diameter tissue-culture treated dishes at 37° C. in a humidified incubator with 5% CO₂ in air. Cells were washed with PBS, incubated with 1×Trypsin and resuspended in DMEM complete. Detached cells were seeded at 6000 cells per well in 50 uL DMEM complete into a 384 well Perkin-Elmer Phenoplate, incubated at room-temperature for 30 minutes followed by incubation overnight at 37° C. in a humidified incubator with 5% CO₂ in air. Recombinant Bax and recombinant cBid were quickly thawed between fingers and 25 mM compound stocks in DMSO were thawed at room temperature. An 8 point titration curve of each compound was created in a 384-well source plate containing Trehalose-Hepes Buffer (THB, 135 mM trehalose, 50 mM KCl, 10 mM HEPES-KOH pH 7.4, 5 mM succinate, 20 uM EDTA, 20 uM EGTA, 0.1% BSA) supplemented with 1/1000 DRAQ5, 0.0025% digitonin, 20 nM Bax and 0.5 nM cBid. The source plate also contained control wells with THB supplemented with 1/1000 DRAQ5, 0.0025% digitonin plus 20 nM Bax (negative control) or 20 nM Bax and 0.5 nM cBid (positive control). Using the Vantage automated liquid handler, DMEM complete media was removed from the cells growing in the 384 well Phenoplate. The cells were washed 2× with 30 uL THB and then 25 uL from the 384 well source plate containing THB proteins and compounds was transferred to the cells growing in the 384 well plate. The cells were incubated for 60-80 minutes at 37 C in a humidified incubator with 5% CO2 in air and then imaged on the Opera Phenix Microscope. Two image channels were acquired: 1) DRAQ5 channel, to visualize nuclei and aid in cell segmentation and 2) SMAC-mCherry channel to visualize mitochondria that retain SMAC-mCherry. Data was processed using PerkinElmer Harmony software where the cells were segmented using the DRAQ5 channel and the mean intensity of SMAC-mCherry for each cell was calculated. The single-cell lobject level data was exported and then analyzed using R code. A per cell SMAC-mCherry intensity cutoff was calculated as 1.33 times the mean per cell SMAC-mCherry intensity of the THB+Bax control. Cells with a SMAC-mCherry intensity below or equal to the cutoff were scored as “released”. The percentage SMAC-mCherry release was calculated as the number of cells scored as “released” divided by the total amount of cells multiplied by 100. Dose-response curves were fit to a 4-parameter log-logistic model to determine the IC₅₀ value for each compound.

Exemplary compounds of the application showed activity in the SMAC mCherry assay having IC₅₀'s in the following ranges: A: 0.1-10 uM; B: 11-50 uM; C: 51-100 uM; D: >100 uM. Specific ranges for exemplary compounds of Formula (I) are shown in Table 2.

TABLE 2

| | | | SMAC |
|---|---|---|---|
| | | | mCherry |
| | | | I.D IC₅₀ |
| Compound | Name | Structures | (uM) |
| 1-1 | N-(1-((3-chloro-4- fluorophenyl)amino)-6- methoxyisoquinolin-7- yl)-4-(piperidin-1- yl)butanamide | 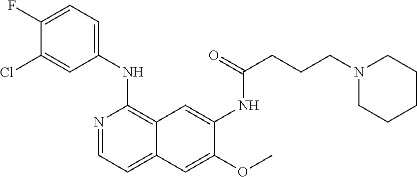 [SMILES: COc1cc2ccnc(Nc3ccc(F)c(Cl)c3)c2cc1NC(=O)CCCN1CCCCC1] | A |
| 1-2 | N-(1-((3-chloro-4- fluorophenyl)amino)-6- methoxyisoquinolin-7- yl)-5-(piperidin-1- yl)pentanamide | 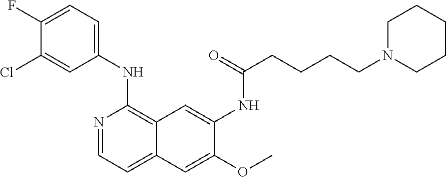 [SMILES: COc1cc2ccnc(Nc3ccc(F)c(Cl)c3)c2cc1NC(=O)CCCCN1CCCCC1] | A |
| 1-3 | N-(1-((3-chloro-4- fluorophenyl)amino)-6- methoxyisoquinolin-7- yl)-3-(piperidin-1- yl)propanamide | 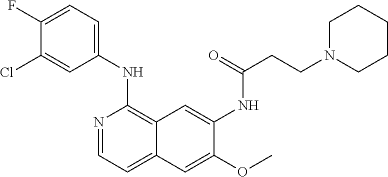 [SMILES: COc1cc2ccnc(Nc3ccc(F)c(Cl)c3)c2cc1NC(=O)CCN1CCCCC1] | B |
| 1-4 | N-(1-((3-chloro-4- fluorophenyl)amino)-6- methoxyisoquinolin-7- yl)-3-(piperidin-1- yl)propanamide | 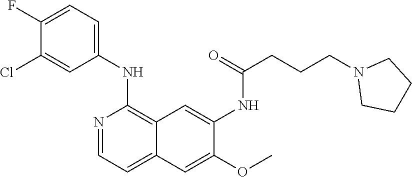 [SMILES: COc1cc2ccnc(Nc3ccc(F)c(Cl)c3)c2cc1NC(=O)CCCN1CCCC1] | A |
| 1-5 | N-(1-((3-chloro-4- fluorophenyl)amino)-6- methoxyisoquinolin-7- yl)-4-((2S,6R)-2,6- dimethylmorpholino) butanamide | 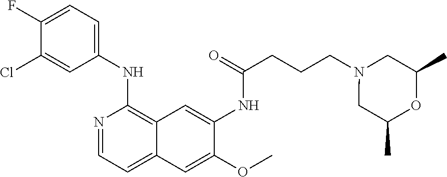 [SMILES: COc1cc2ccnc(Nc3ccc(F)c(Cl)c3)c2cc1NC(=O)CCCN1C[C@@H](C)O[C@@H](C)C1] | A |
| 1-6 | N-(1-((3-chloro-4- fluorophenyl)amino)-6- methoxyisoquinolin-7- yl)-4- morpholinobutanamide | 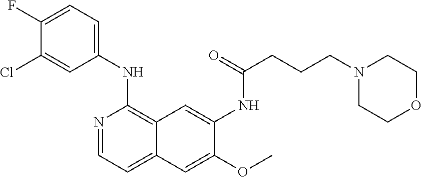 [SMILES: COc1cc2ccnc(Nc3ccc(F)c(Cl)c3)c2cc1NC(=O)CCCN1CCOCC1] | B |
| 1-7 | N-(1-((3-chloro-4- fluorophenyl)amino)-6- methoxyisoquinolin-7- yl)-4-(4- methylpiperazin-1- yl)butanamide | 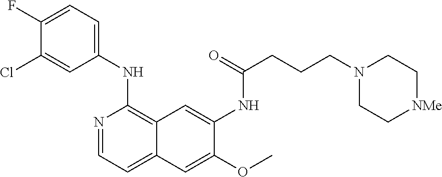 [SMILES: COc1cc2ccnc(Nc3ccc(F)c(Cl)c3)c2cc1NC(=O)CCCN1CCN(C)CC1] | A |
| 1-8 | N-(1-((3-chloro-4- fluorophenyl)amino)-6- methoxyisoquinolin-7- yl)-4-(4,4- difluoropiperidin-1- yl)butanamide | 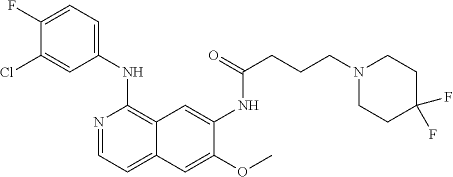 [SMILES: COc1cc2ccnc(Nc3ccc(F)c(Cl)c3)c2cc1NC(=O)CCCN1CCC(F)(F)CC1] | A |
| 1-9 | N-(1-((3- fluorophenyl)amino)-6- methoxyisoquinolin-7- yl)-4-(piperidin-1- yl)butanamide | 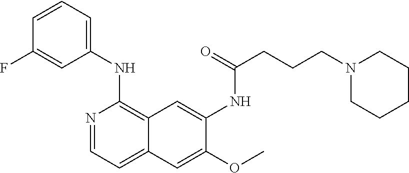 [SMILES: COc1cc2ccnc(Nc3cccc(F)c3)c2cc1NC(=O)CCCN1CCCCC1] | B |
| 1-10 | N-(6-methoxy-1-((3- methoxyphenyl)amino) isoquinolin-7-yl)-4- (piperidin-1- yl)butanamide | 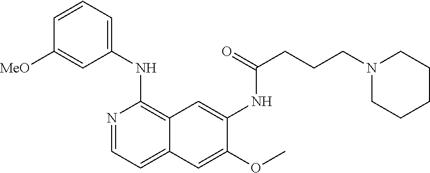 [SMILES: COc1cccc(Nc2nccc3cc(OC)c(NC(=O)CCCN4CCCCC4)cc23)c1] | D |
| 1-11 | N-(6-methoxy-1-(m- tolylamino)isoquinolin- 7-yl)-4-(piperidin-1- yl)butanamide | 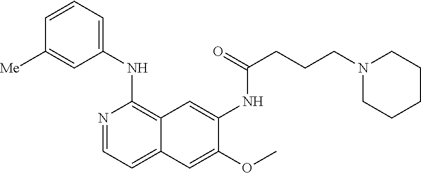 [SMILES: COc1cc2ccnc(Nc3cccc(C)c3)c2cc1NC(=O)CCCN1CCCCC1] | C |
| 1-12 | N-(1-((4- fluorophenyl)amino)-6- methoxyisoquinolin-7- yl)-4-(piperidin-1- yl)butanamide | 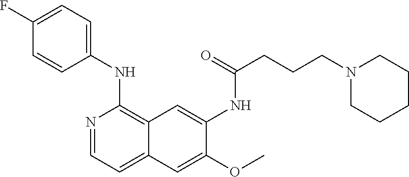 [SMILES: COc1cc2ccnc(Nc3ccc(F)cc3)c2cc1NC(=O)CCCN1CCCCC1] | C |
| 1-13 | N-(1-((3- chlorophenyl)amino)- 6-methoxyisoquinolin- 7-yl)-4-(piperidin-1- yl)butanamide | 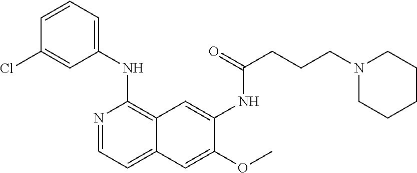 [SMILES: COc1cc2ccnc(Nc3cccc(Cl)c3)c2cc1NC(=O)CCCN1CCCCC1] | B |
| 1-14 | N-(6-methoxy-1-(p- tolylamino)isoquinolin- 7-yl)-4-(piperidin-1- yl)butanamide | 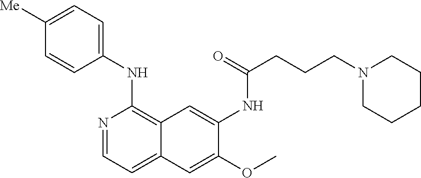 [SMILES: COc1cc2ccnc(Nc3ccc(C)cc3)c2cc1NC(=O)CCCN1CCCCC1] | C |
| 1-15 | N-(6-methoxy-1- (phenylamino)isoquinoin- l7-yl)-4-(piperidin-1- yl)butanamide | 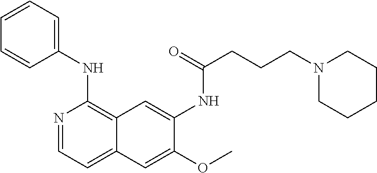 [SMILES: COc1cc2ccnc(Nc3ccccc3)c2cc1NC(=O)CCCN1CCCCC1] | C |
| 1-16 | N-(1-((4-fluoro-3- (trifluoromethyl) phenyl)amino)-6- methoxyisoquinolin-7- yl)-4-(piperidin-1- yl)butanamide | 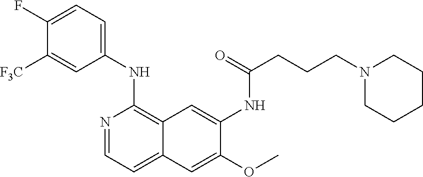 [SMILES: COc1cc2ccnc(Nc3ccc(F)c(C(F)(F)F)c3)c2cc1NC(=O)CCCN1CCCCC1] | A |
| 1-17 | N-(1-((3-chloro-4- fluorophenyl)amino)-6- methylisoquinolin-7- yl)-4-(piperidin-1- yl)butanamide | 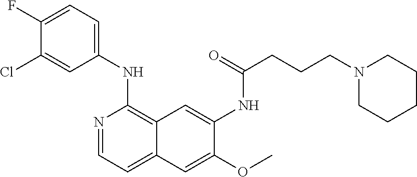 [SMILES: COc1cc2ccnc(Nc3ccc(F)c(Cl)c3)c2cc1NC(=O)CCCN1CCCCC1] | B |
| 1-18 | N-(1-((3-chloro-4- fluorophenyl)amino) isoquinolin-7-yl)-4- (piperidin-1- yl)butanamide | 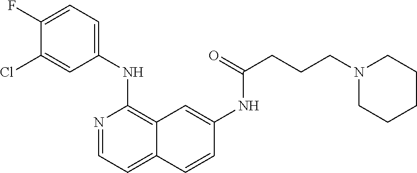 [SMILES: O=C(CCCN1CCCCC1)Nc1ccc2ccnc(Nc3ccc(F)c(Cl)c3)c2c1] | A |
| I-19 | N-(1-((2-chloro-4- fluorophenyl)amino)-6- methoxyisoquinolin-7- yl)-4-(piperidin-1- yl)butanamide | 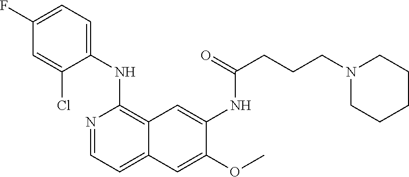 [SMILES: COc1cc2ccnc(Nc3ccc(F)cc3Cl)c2cc1NC(=O)CCCN1CCCCC1] | B |
| 1-20 | N-(1-((4-fluoro-3- methylphenyl)amino)- 6-methoxyisoquinolin- 7-yl)-4-(piperidin-1- yl)butanamide | 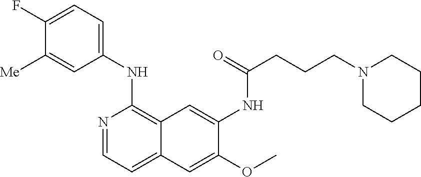 [SMILES: COc1cc2ccnc(Nc3ccc(F)c(C)c3)c2cc1NC(=O)CCCN1CCCCC1] | |
| 1-21 | N-(1-((3-chloro-4- methylphenyl)amino)- 6-methoxyisoquinolin- 7-yl)-4-(piperidin-1- yl)butanamide | 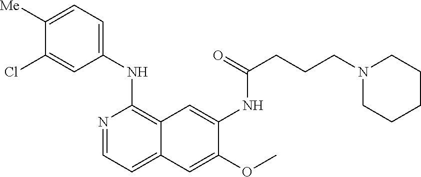 [SMILES: COc1cc2ccnc(Nc3ccc(C)c(Cl)c3)c2cc1NC(=O)CCCN1CCCCC1] | A |
| 1-22 | N-(1-((4-fluoro-3- methoxyphenyl)amino)- 6- methoxyisoquinolin-7- yl)-4-(piperidin-1- yl)butanamide | 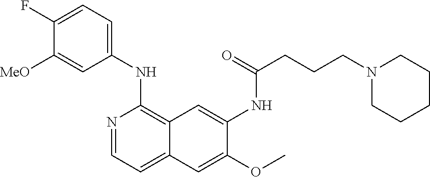 [SMILES: COc1cc(Nc2nccc3cc(OC)c(NC(=O)CCCN4CCCCC4)cc23)ccc1F] | B |
| 1-23 | N-(1-((3,5-dichloro-4- fluorophenyl)amino)-6- methoxyisoquinolin-7- yl)-4-(piperidin-1- yl)butanamide | 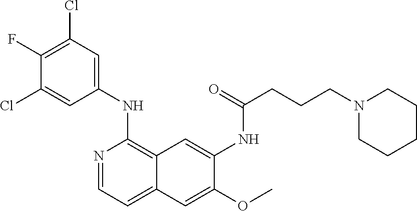 [SMILES: COc1cc2ccnc(Nc3cc(Cl)c(F)c(Cl)c3)c2cc1NC(=O)CCCN1CCCCC1] | A |
| 1-24 | N-(1-((3-chloro-4- methoxyphenyl)amino)- 6- methoxyisoquinolin-7- yl)-4-(piperidin-1- yl)butanamide | 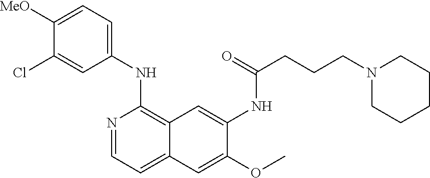 [SMILES: COc1ccc(Nc2nccc3cc(OC)c(NC(=O)CCCN4CCCCC4)cc23)cc1Cl] | B |
| 1-25 | N-(1-((3-chloro-5- fluorophenyl)amino)-6- methoxyisoquinolin-7- yl)-4-(piperidin-1- yl)butanamide | 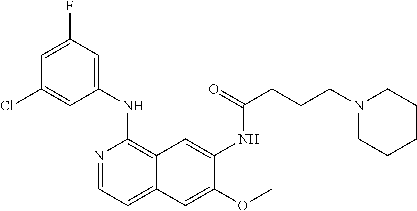 [SMILES: COc1cc2ccnc(Nc3cc(F)cc(Cl)c3)c2cc1NC(=O)CCCN1CCCCC1] | A |
| 1-26 | N-(6-chloro-1-((3- chloro-4- fluorophenyl)amino) isoquinolin-7-yl)-4- (piperidin-1- yl)butanamide | 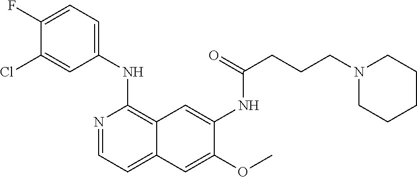 [SMILES: COc1cc2ccnc(Nc3ccc(F)c(Cl)c3)c2cc1NC(=O)CCCN1CCCCC1] | A |
| 1-27 | N-(1-((3,4- dichlorophenyl)amino)- 6-methoxyisoquinolin- 7-yl)-4-(piperidin-1- yl)butanamide | 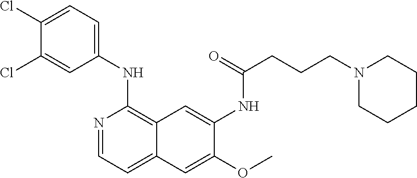 [SMILES: COc1cc2ccnc(Nc3ccc(Cl)c(Cl)c3)c2cc1NC(=O)CCCN1CCCCC1] | A |
| 1-28 | N-(1-((3-ethyl-4- fluorophenyl)amino)-6- methoxyisoquinolin-7- yl)-4-(piperidin-1- yl)butanamide | 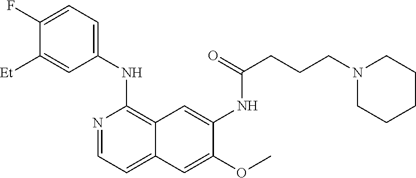 [SMILES: CCc1cc(Nc2nccc3cc(OC)c(NC(=O)CCCN4CCCCC4)cc23)ccc1F] | B |
| 1-29 | N-(1-((3-chloro-4- fluorophenyl)amino)-5- methoxyisoquinolin-7- yl)-4-(piperidin-1- yl)butanamide | 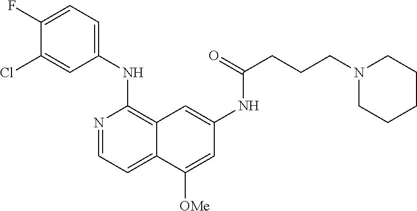 [SMILES: COc1cc(NC(=O)CCCN2CCCCC2)cc2c(Nc3ccc(F)c(Cl)c3)nccc12] | A |
| 1-30 | N-(1-((4-fluoro-3- isopropylphenyl)amino)- 6- methoxyisoquinolin-7- yl)-4-(piperidin-1- yl)butanamide | 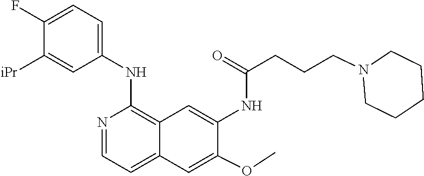 [SMILES: COc1cc2ccnc(Nc3ccc(F)c(C(C)C)c3)c2cc1NC(=O)CCCN1CCCCC1] | B |
| 1-31 | N-(1-((3-chloro-4- fluorophenyl)amino)-6- methoxyisoquinolin-7- yl)-N-methyl-4- (piperidin-1- yl)butanamide | 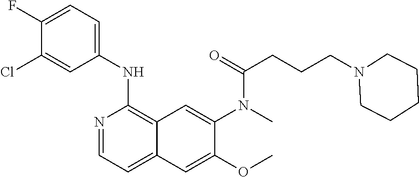 [SMILES: COc1cc2ccnc(Nc3ccc(F)c(Cl)c3)c2cc1N(C)C(=O)CCCN1CCCCC1] | B |
| 1-32 | N-(1-((3-chloro-4- methylphenyl)amino) isoquinolin-7-yl)-4- (piperidin-1- yl)butanamide | 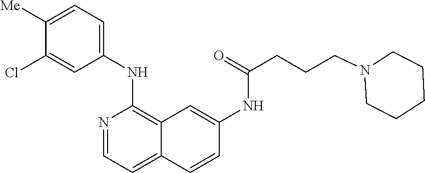 [SMILES: Cc1ccc(Nc2nccc3ccc(NC(=O)CCCN4CCCCC4)cc23)cc1Cl] | A |
| 1-33 | N-(1-((4-fluoro-3- (trifluoromethyl)phenyl )amino)isoquinolin-7- yl)-4-(piperidin-1- yl)butanamide | 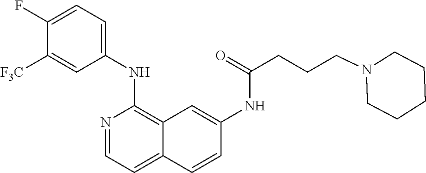 [SMILES: O=C(CCCN1CCCCC1)Nc1ccc2ccnc(Nc3ccc(F)c(C(F)(F)F)c3)c2c1] | A |
| 1-34 | N-(1-((3,4-dichloro-2- fluorophenyl)amino) isoquinolin-7-yl)-4- (piperidin-1- yl)butanamide | 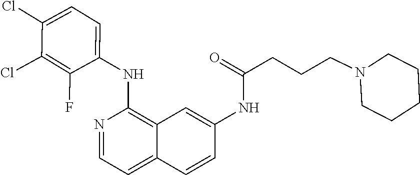 [SMILES: O=C(CCCN1CCCCC1)Nc1ccc2ccnc(Nc3ccc(Cl)c(Cl)c3F)c2c1] | A |
| 1-35 | N-(1-((3,4- dichlorophenyl)amino) isoquinolin-7-yl)-4- (piperidin-1- yl)butanamide | 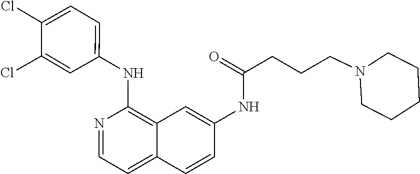 [SMILES: O=C(CCCN1CCCCC1)Nc1ccc2ccnc(Nc3ccc(Cl)c(Cl)c3)c2c1] | A |
| 1-36 | N-(1-((3,5-dichloro-4- fluorophenyl)amino) isoquinolin-7-yl)-4- (piperidin-1- yl)butanamide | 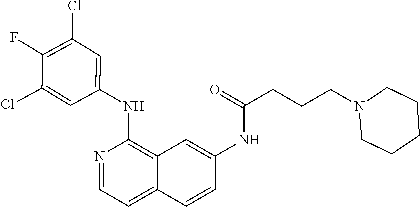 [SMILES: O=C(CCCN1CCCCC1)Nc1ccc2ccnc(Nc3cc(Cl)c(F)c(Cl)c3)c2c1] | A |
| 1-37 | N-(1-((3,4-dichloro-5- fluorophenyl)amino) isoquinolin-7-yl)-4- (piperidin-1- yl)butanamide | 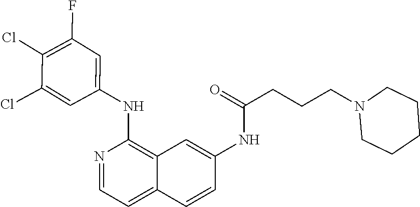 [SMILES: O=C(CCCN1CCCCC1)Nc1ccc2ccnc(Nc3cc(F)c(Cl)c(Cl)c3)c2c1] | A |
| 1-38 | N-(1-((4,5-dichloro-2- fluorophenyl)amino) isoquinolin-7-yl)-4- (piperidin-1- yl)butanamide | 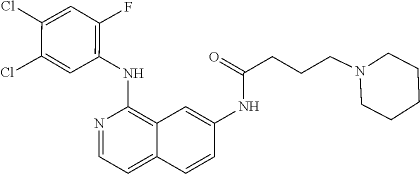 [SMILES: O=C(CCCN1CCCCC1)Nc1ccc2ccnc(Nc3cc(Cl)c(Cl)cc3F)c2c1] | A |
| 1-39 | N-(1-((3-chloro-2- fluorophenyl)amino) isoquinolin-7-yl)-4- (piperidin-1- yl)butanamide | 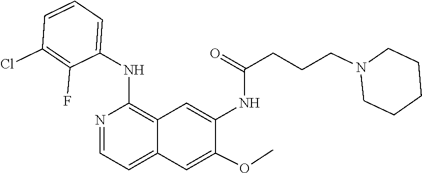 [SMILES: COc1cc2ccnc(Nc3cccc(Cl)c3F)c2cc1NC(=O)CCCN1CCCCC1] | A |
| 1-40 | N-(1-((3-chloro-2,4- difluorophenyl)amino) isoquinolin-7-yl)-4- (piperidin-1- yl)butanamide | 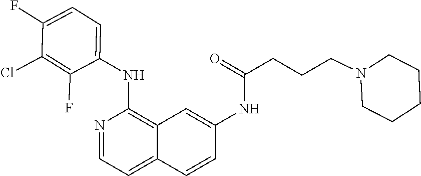 [SMILES: O=C(CCCN1CCCCC1)Nc1ccc2ccnc(Nc3ccc(F)c(Cl)c3F)c2c1] | A |
| 1-41 | N-(1-((3- cyanophenyl)amino) isoquinolin-7-yl)-4- (piperidin-1- yl)butanamide | 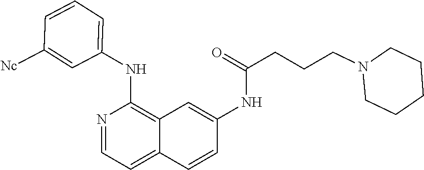 | B |
| 1-42 | N-(1-((3-chloro-4- fluorophenyl)amino)-6- methoxyisoquinolin-7- yl)-3-(pyrrolidin-1- yl)propanamide | 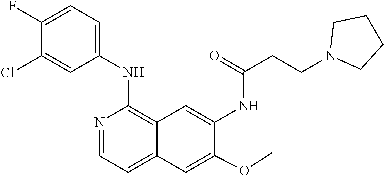 [SMILES: COc1cc2ccnc(Nc3ccc(F)c(Cl)c3)c2cc1NC(=O)CCN1CCCC1] | B |
| 1-43 | N-(1-((3-chloro-4- fluorophenyl)amino)-6- methoxyisoquinolin-7- yl)-4-(3,3- difluoropyrrolidin-1- yl)butanamide | 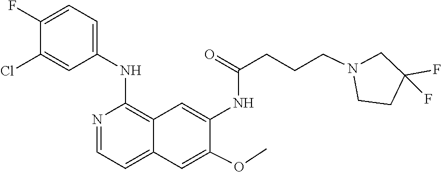 [SMILES: COc1cc2ccnc(Nc3ccc(F)c(Cl)c3)c2cc1NC(=O)CCCN1CCC(F)(F)C1] | |
| 1-44 | N-(1-((3,5-dichloro-2- fluorophenyl)amino) isoquinolin-7-yl)-4- (piperidin-1- yl)butanamide | 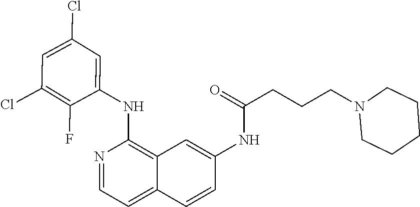 [SMILES: O=C(CCCN1CCCCC1)Nc1ccc2ccnc(Nc3cc(Cl)cc(Cl)c3F)c2c1] | |
| 1-45 | N-(1-((4-chloro-2,3- difluorophenyl)amino) isoquinolin-7-yl)-4- (piperidin-1- yl)butanamide | 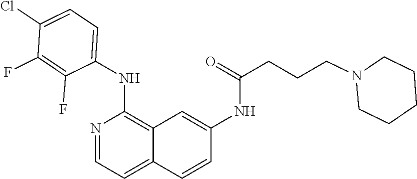 [SMILES: O=C(CCCN1CCCCC1)Nc1ccc2ccnc(Nc3ccc(Cl)c(F)c3F)c2c1] | |
| 1-46 | N-(1-((3-chloro-4- fluorophenyl)amino)-6- ethoxyisoquinolin-7- yl)-4-(piperidin-1- yl)butanamide | 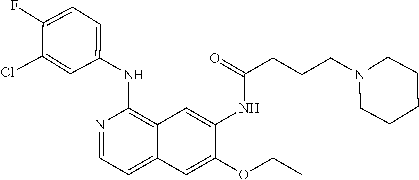 [SMILES: CCOc1cc2ccnc(Nc3ccc(F)c(Cl)c3)c2cc1NC(=O)CCCN1CCCCC1] | |
| (R) I-47 | (R)-N-(1-((3-chloro-4- fluorophenyl)amino)-6- methoxyisoquinolin-7- yl)-4-(3- methoxypyrrolidin-1- yl)butanamide | 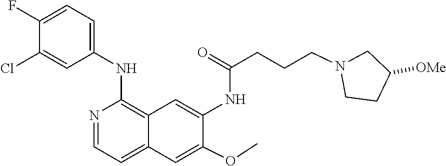 [SMILES: COc1cc2ccnc(Nc3ccc(F)c(Cl)c3)c2cc1NC(=O)CCCN1CC[C@@H](OC)C1] | |
| (S) 1-47 | (S)-N-(1-((3-chloro-4- fluorophenyl)amino)-6- methoxyisoquinolin-7- yl)-4-(3- methoxypyrrolidin-1- yl)butanamide | 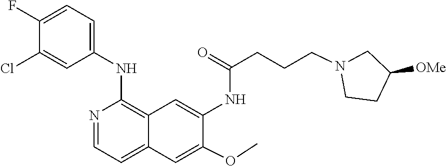 [SMILES: COc1cc2ccnc(Nc3ccc(F)c(Cl)c3)c2cc1NC(=O)CCCN1CC[C@H](OC)C1] | |
| (R) I-48 | (R)-N-(1-((3-chloro-4- fluorophenyl)amino)-6- methoxyisoquinolin-7- yl)-4-(3- fluoropyrrolidin-1- yl)butanamide | 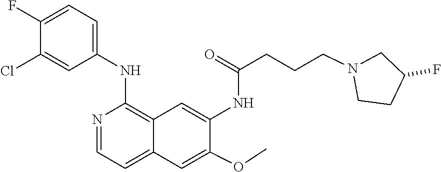 [SMILES: COc1cc2ccnc(Nc3ccc(F)c(Cl)c3)c2cc1NC(=O)CCCN1CC[C@@H](F)C1] | |

A: 0.1-10 uM; B: 11-50 uM; C: 51-100 uM; D: >100 uM.

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

A number of publications are cited herein. Full citations for these references are provided below. Each of these references is incorporated herein by reference in its entirety into the present disclosure, to the same extent as if each individual reference was specifically and individually indicated to be incorporated by reference.
Green, D. R. and Kroemer, G. (2004) ‘The Pathophysiology of Mitochondrial Cell Death’, Science, 305(5684), pp. 626-629. doi: 10.1126/science.1099320
Arden, N. and Betenbaugh, M. J. (2004) ‘Life and death in mammalian cell culture: strategies for apoptosis inhibition’, Trends in Biotechnology, 22(4), pp. 174-180. doi: 10.1016/j.tibtech.2004.02.004.
Mergenthaler, P., Dirnagl, U. and Meisel, A. (2004) ‘Pathophysiology of stroke: Lessons from animal models’, Metabolic Brain Disease, 19(3-4), pp. 151-167. doi: 10.1023/B:MEBR.0000043966.46964.e6.
Leber, B., Geng, F., Kale, J. and Andrews, D. W. (2010) ‘Drugs targeting Bcl-2 family members as an emerging strategy in cancer.’, Expert reviews in molecular medicine. University of Toronto, 12 (September), p. e28. doi: 10.1017/S1462399410001572
Octavia, Y., Tocchetti, C. G., Gabrielson, K. L., Janssens, S., Crijns, H. J. and Moens, A. L. (2012) ‘Doxorubicin-induced cardiomyopathy: From molecular mechanisms to therapeutic strategies’, Journal of Molecular and Cellular Cardiology. Elsevier Ltd, 52(6), pp. 1213-1225. doi: 10.1016/j.yjmcc.2012.03.006
Pang, A. L., Xiong, L. L., Xia, Q. J., Liu, Fen, Wang, Y. C., Liu, Fei, Zhang, P., Meng, B. L., Tan, S. and Wang, T. H. (2017) ‘Neural stem cell transplantation is associated with inhibition of apoptosis, Bcl-xL upregulation, and recovery of neurological function in a rat model of traumatic brain injury’, Cell Transplantation, 26(7), pp. 1262-1275. doi: 10.1177/0963689717715168
Afreen, S., Weiss, J. M., Strahm, B. and Erlacher, M. (2018) ‘Concise Review: Cheating Death for a Better Transplant’, STEM CELLS, 36(11), pp. 1646-1654. doi: 10.1002/stem.2901
Pemberton, J. M., Pogmore, J. P. and Andrews, D. W. (2020) ‘Neuronal cell life, death, and axonal degeneration as regulated by the BCL-2 family proteins’, Cell Death & Differentiation. Springer US. doi: 10.1038/s41418-020-00654-2
Kale, J.; Osterlund, E. J.; Andrews, D. W. BCL-2 Family Proteins: Changing Partners in the Dance towards Death. Cell Death Differ. 2018, 25 (1), 65-80.
Aouacheria, A.; Combet, C.; Tompa, P.; Hardwick, J. M. Redefining the BH3 Death Domain as a ‘Short Linear Motif.’. Trends Biochem. Sci. 2015, 40 (12), 736-748
Leveille, F.; Papadia, S.; Fricker, M.; Bell, K. F. S.; Soriano, F. X.; Martel, M. A.; Puddifoot, C.; Habel, M.; Wyllie, D. J.; Ikonomidou, C.; Tolkovsky, A. M.; Hardingham, G. E. Suppression of the Intrinsic Apoptosis Pathway by Synaptic Activity. J. Neurosci. 2010, 30 (7), 2623-2635.
Chi, X.; Nguyen, D.; Pemberton, J. M.; Osterlund, E. J.; Liu, Q.; Brahmbhatt, H.; Zhang, Z.; Lin, J.; Leber, B.; Andrews, D. W. The Carboxyl-Terminal Sequence of Bim Enables Bax Activation and Killing of Unprimed Cells. Elife 2020, 9, 1-31.
Lovell, J. F.; Billen, L. P.; Bindner, S.; Shamas-Din, A.; Fradin, C.; Leber, B.; Andrews, D. W. Membrane Binding by TBid Initiates an Ordered Series of Events Culminating in Membrane Permeabilization by Bax. Cell 2008, 135 (6), 1074-1084.
Fernández-Marrero, Y.; Bleicken, S.; Das, K. K.; Bachmann, D.; Kaufmann, T.; Garcia-Saez, A. J. The Membrane Activity of BOK Involves Formation of Large, Stable Toroidal Pores and Is Promoted by CBID. FEBS J. 2017, 284, 711-724.
Hockings, C.; Anwari, K.; Ninnis, R. L.; Brouwer, J.; O'Helv, M.; Evangelista, M.; Hinds, M. G.; Czabotar, P. E.; Lee, E. F.; Fairlie, W. D.; Dewson, G.; Kluck, R. M. Bid Chimeras Indicate That Most BH3-Only Proteins Can Directly Activate Bak and Bax, and Show No Preference for Bak versus Bax. Cell Death Dis. 2015, 6, No. e1735.
Kuwana, T.; Bouchier-Hayes, L.; Chipuk, J. E.; Bonzon, C.; Sullivan, B. A.; Green, D. R.; Newmeyer, D. D. BH3 Domains of BH3-Only Proteins Differentially Regulate Bax-Mediated Mitochondrial Membrane Permeabilization Both Directly and Indirectly. Mol. Cell 2005, 17 (4), 525-535.
Kim, H., Tu, H.-C., Ren, D., Takeuchi, O., Jeffers, J. R., Zambetti, G. P., Hsieh, J. J.-D. and Cheng, E. H.-Y. (2009) ‘Stepwise Activation of BAX and BAK by tBID, BIM, and PUMA Initiates Mitochondrial Apoptosis’, Molecular Cell, 36(3), pp. 487-499. doi: 10.1016/j.molcel.2009.09.030
Llambi, F., Moldoveanu, T., Tait, S. W. G., Bouchier-Hayes, L., Temirov, J., McCormick, L. L., Dillon, C. P. and Green, D. R. (2011) ‘A Unified Model of Mammalian BCL-2 Protein Family Interactions at the Mitochondria’, Molecular Cell, 44(4), pp. 517-531. doi: 10.1016/j.molcel.2011.10.001
Shamas-Din, A., Kale, J., Leber, B. and Andrews, D. W. (2013) ‘Mechanisms of action of Bcl-2 family proteins’, Cold Spring Harbor Perspectives in Biology, 5(4), pp. 1-21. doi: 10.1101/cshperspect.a008714
Tait, S. W. G. and Green, D. R. (2010) ‘Mitochondria and cell death: Outer membrane permeabilization and beyond’, Nature Reviews Molecular Cell Biology. Nature Publishing Group, 11(9), pp. 621-632. doi: 10.1038/nrm2952.
Westphal, D., Dewson, G., Czabotar, P. E. and Kluck, R. M. (2011) ‘Molecular biology of Bax and Bak activation and action.’, Biochimica et biophysica acta. Elsevier B. V., 1813(4), pp. 521-31. doi: 10.1016/j.bbamcr.2010.12.019
Hill, R. B., MacKenzie, K. R. and Harwig, M. C. (2015) ‘The Tail-End Is Only the Beginning: NMR Study Reveals a Membrane-Bound State of BCL-XL.’, Journal of molecular biology. Elsevier B.V., 427(13), pp. 2257-61. doi: 10.1016/j.jmb.2015.04.005.
Niu, X., Brahmbhatt, H., Mergenthaler, P., Zhang, Z., Sang, J., Daude, M., Ehlert, F. G. R., Diederich, W. E., Wong, E., Zhu, W., Pogmore, J., Nandy, J. P., Satyanarayana, M., Jimmidi, R. K., Arya, P., Leber, B., Lin, J., Culmsee, C., Yi, J. and Andrews, D. W. (2017) ‘A Small-Molecule Inhibitor of Bax and Bak Oligomerization Prevents Genotoxic Cell Death and Promotes Neuroprotection’, Cell Chemical Biology. Elsevier, 24(4), pp. 493-506.e5. doi: 10.1016/j.chembiol.2017.03.011.
Amgalan, D.; Garner, T. P.; Pekson, R.; Jia, X. F.; Yanamandala, M.; Paulino, V.; Liang, F. G.; Corbalan, J. J.; Lee, J.; Chen, Y.; Karagiannis, G. S.; Sanchez, L. R.; Liang, H.; Narayanagari, S.; Mitchell, K.; Lopez, A.; Margulets, V.; Scarlata, M.; Santulli, G.; Asnani, A.; Peterson, R. T.; Hazan, R. B.; Condeelis, J. S.; Oktay, M. H.; Steidl, U.; Kirshenbaum, L. A.; Gavathiotis, E.; Kitsis, R. N. A Small-Molecule Allosteric Inhibitor of BAX Protects against Doxorubicin-Induced Cardiomyopathy. Nat. cancer 2020, 1 (3),
Pogmore, J. P., Uehling, D. and Andrews, D. W. (2021) ‘Pharmacological Targeting of Executioner Proteins: Controlling Life and Death.’, Journal of medicinal chemistry, 64(9), pp. 5276-5290. doi: 10.1021/acs.jmedchem.0c02200
Kale, J., Chi, X., Leber, B. and Andrews, D. (2014) ‘Examining the Molecular Mechanism of Bcl-2 Family Proteins at Membranes by Fluorescence Spectroscopy’, in Methods in Enzymology. 1st edn. Elsevier Inc., pp. 1-23. doi: 10.1016/B978-0-12-417158-9.00001-7.
Pogmore, J. P., Pemberton, J. M., Chi, X. and Andrews, D. W. (2016) Using Förster-Resonance Energy Transfer to Measure Protein Interactions Between Bcl-2 Family Proteins on Mitochondrial Membranes, Methods in Molecular Biology. Edited by H. Puthalakath and C. J. Hawkins. New York, NY: Springer New York (Methods in Molecular Biology). doi: 10.1007/978-1-4939-3581-9
Moldoveanu, T., Liu, Q., Tocilj, A., Watson, M., Shore, G. and Gehring, K. (2006) ‘The X-Ray Structure of a BAK Homodimer Reveals an Inhibitory Zinc Binding Site’, Molecular Cell. Elsevier Inc., 24(5), pp. 677-688. doi: 10.1016/j.molcel.2006.10.014
Liu, Q., Osterlund, E. J., Chi, X., Pogmore, J., Leber, B. and Andrews, D. W. (2019) ‘Bim escapes displacement by BH3-mimetic anti-cancer drugs by double-bolt locking both Bcl-XL and Bcl-2’, eLife, 8, pp. 1-30. doi: 10.7554/eLife.37689
Bogner, C., Kale, J., Pogmore, J., Chi, X., Shamas-Din, A., Fradin, C., Leber, B. and Andrews, D. W. (2020) ‘Allosteric Regulation of BH3 Proteins in Bcl-xL Complexes Enables Switch-like Activation of Bax’, Molecular Cell, 77(4), pp. 901-912.e9. doi: 10.1016/j.molcel.2019.12.02
Gavathiotis, E., Reyna, D. E., Davis, M. L., Bird, G. H. and Walensky, L. D. (2010a) ‘BH3-Triggered Structural Reorganization Drives the Activation of Proapoptotic BAX’, Molecular Cell, 40(3), pp. 481-492. doi: 10.1016/j.molcel.2010.10.019
Gavathiotis, E., Reyna, D. E., Davis, M. L., Bird, G. H. and Walensky, L. D. (2010b) ‘BH3-Triggered Structural Reorganization Drives the Activation of Proapoptotic BAX’, Molecular Cell. Elsevier Inc., 40(3), pp. 481-492. doi: 10.1016/j.molcel.2010.10.019
Bleicken, S., Landeta, O., Landajuela, A., Basanez, G. and Garcia-Saez, A. J. (2013) ‘Proapoptotic Bax and Bak Proteins Form Stable Protein-permeable Pores of Tunable Size’, Journal of Biological Chemistry, 288(46), pp. 33241-33252. doi: 10.1074/jbc.M1 13.512087
Czabotar, P. E., Westphal, D., Dewson, G., Ma, S., Hockings, C., Fairlie, W. D., Lee, E. F., Yao, S., Robin, A. Y., Smith, B. J., Huang, D. C. S., Kluck, R. M., Adams, J. M. and Colman, P. M. (2013) ‘Bax Crystal Structures Reveal How BH3 Domains Activate Bax and Nucleate Its Oligomerization to Induce Apoptosis’, Cell. Elsevier Inc., 152(3), pp. 519-531. doi: 10.1016/j.cell.2012.12.031
Brouwer, J. M., Westphal, D., Dewson, G., Robin, A. Y., Uren, R. T., Bartolo, R., Thompson, G. V, Colman, P. M., Kluck, R. M. and Czabotar, P. E. (2014) ‘Bak core and latch domains separate during activation, and freed core domains form symmetric homodimers.’, Molecular cell. Elsevier Inc., 55(6), pp. 938-46. doi: 10.1016/j.molcel.2014.07.016
Roskoski, R. (2016) ‘Classification of small molecule protein kinase inhibitors based upon the structures of their drug-enzyme complexes’, Pharmacological Research. Elsevier Ltd, 103, pp. 26-48. doi: 10.1016/j.phrs.2015.10.02
Fry, D. W., Bridges, A. J., Denny, W. A., Doherty, A., Greis, K. D., Hicks, J. L., Hook, K. E., Keller, P. R., Leopold, W. R., Loo, J. A., McNamara, D. J., Nelson, J. M., Sherwood, V., Smaill, J. B., Trumpp-Kallmeyer, S. and Dobrusin, E. M. (1998) ‘Specific, irreversible inactivation of the epidermal growth factor receptor and erbB2, by a new class of tyrosine kinase inhibitor’, Proceedings of the National Academy of Sciences, 95(20), pp. 12022-12027. doi: 10.1073/pnas.95.20.12022
Tang, Z., Jiang, X., Guo, X., Man, C., Fong, V. and Lu, J. (2016) ‘Characterization of osimertinib (AZD9291)-resistant non-small cell lung cancer NCl—H1975/OSIR cell line’, 7(49), pp. 81598-81610
Momeny, M., Zarrinrad, G., Moghaddaskho, F., Poursheikhani, A., Sankanian, G., Zaghal, A., Mirshahvaladi, S., Esmaeili, F., Eyvani, H., Barghi, F., Sabourinejad, Z., Alishahi, Z., Yousefi, H., Ghasemi, R., Dardaei, L., Bashash, D., Chahardouli, B., Dehpour, A. R., Tavakkoly-Bazzaz, J., Alimoghaddam, K., Ghavamzadeh, A. and Ghaffari, S. H. (2017) ‘Dacomitinib, a pan-inhibitor of ErbB receptors, suppresses growth and invasive capacity of chemoresistant ovarian carcinoma cells’, Scientific Reports, 7(1), pp. 1-12. doi: 10.1038/s41598-017-04147-0.

1. A compound of Formula I, or a pharmaceutically acceptable salt, solvate and/or prodrug thereof: 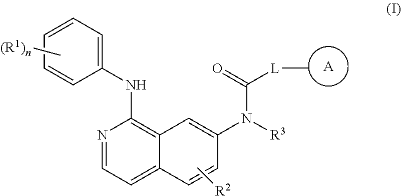
wherein
each R¹ is independently selected from halo, CN, C₁₋₄alkyl, C₁₋₄haloalkyl, OC₁₋₄alkyl and OC₁₋₄haloalkyl;
R² is absent, or R² is selected from halo, C₁₋₄alkyl, C₁₋₄haloalkyl, OC₁₋₄alkyl and OC₁₋₄haloalkyl;
R³ is selected from H and C₁₋₄alkyl;
L is selected from C₁₋₄alkylene, C₂₋₄alkenylene and C₂₋₄alkynylene;
A is C₃₋₈heterocycloalkyl including at least one ring heteromoiety selected from N, NH and N(C₁₋₄alkyl), and optionally substituted with one or two substituents selected from OH, halo, C₁₋₂alkyl, C₁₋₂haloalkyl, OC₁₋₂alkyl and OC₁₋₂haloalkyl; and
n is selected from 0, 1, 2 and 3.

2. The compound of claim 1, wherein each R¹ is independently selected from F, Cl, Br, CN, C₁₋₄alkyl, C₁₋₄haloalkyl, OC₁₋₄alkyl and OC₁₋₄haloalkyl.

3. The compound of claim 2, wherein each R¹ is independently selected from F, Cl, Br, CN, CH₃, CH₂CH₃, CH₂CH₂CH₃, CH(CH₃)₂, CF₃, CHF₂, CFH₂, CH₂CHF₂, CH₂CF₃, CH₂CFH₂, CCl₃, CH₂CClH₂, CCl₂H, CH₂CCl₂H, CH₂CCl₃, OCH₃, OCH₂CH₃, OCH₂CH₂CH₃, OCH(CH₃)₂, OCF₃, OCHF₂, OCH₂CHF₂, OCH₂CF₃, OCH₂CFH₂, OCCl₃, OCH₂CClH₂, OCCl₂H, OCH₂CCl₂H and OCH₂CCl₃.

4. The compound of claim 4, wherein each R¹ is independently selected from F, Cl, CN, CH₃, CH₂CH₃, CH₂CH₂CH₃, CH(CH₃)₂, CF₃, CHF₂, CCl₃, CCl₂H, OCH₃, OCF₃, OCHF₂, OCCl₃ and OCCl₂H.

5. The compound of claim 4, wherein each R¹ is CN.

6. The compound of claim 4, wherein each R¹ is independently selected from F, Cl, OCH₃ and OCF₃.

7. The compound of claim 4, wherein each R¹ is independently selected from F, Cl, CH₃, CF₃, CH₂CH₃, CH₂CH₂CH₃ and CH(CH₃)₂.

8. The compound of claim 4, wherein each R¹ is independently selected from F and Cl.

9. The compound of any one of claims 1 to 8, wherein n is 2 or 3.

10. The compound of claim 9, wherein n is 2.

11. The compound of claim 1, wherein n is 0.

12. The compound of any one of claims 1 to 11, wherein R² is selected from F, Br, Cl, C₁₋₄alkyl, C₁₋₄haloalkyl, OC₁₋₄alkyl and OC₁₋₄haloalkyl.

13. The compound of claim 12, wherein R² is selected from F, Br, Cl, C₁₋₃alkyl, C₁₋₃fluoroalkyl, C₁₋₃chloroalkyl, OC₁₋₃alkyl, OC₁₋₃fluoroalkyl and OC₁₋₃chloroalkyl.

14. The compound of claim 13, wherein R² is selected from F, C, Br, CH₃, CH₂CH₃, CF₃, CHF₂, CCl₃, CCl₂H, OCH₃, OCH₂CH₃, OCH₂CH₂CH₃, OCH(CH₃)₂, OCF₃, OCHF₂, OCH₂CHF₂, OCH₂CF₃, OCH₂CFH₂, OCCl₃, OCH₂CClH₂, OCCl₂H, OCH₂CCl₂H and OCH₂CCl₃.

15. The compound of claim 14, wherein R² is selected from F, C, CH₃, CF₃, OCH₃, OCH₂CH₃, OCH₂CH₂CH₃, OCH(CH₃)₂, OCF₃ and OCHF₂.

16. The compound of claim 15, wherein R² is OCH₃.

17. The compound of any one of claims 1 to 11, wherein R² is absent.

18. The compound of any one of claims 1 to 17, wherein R³ is selected from H and CH₃.

19. The compound of claim 18, wherein R³ is H.

20. The compound of any one of claims 1 to 19, wherein L is selected from C₁₋₄alkylene and C₂₋₄alkenylene.

21. The compound of claim 20, wherein L is selected from CH₂CH₂CH₂ (C₃alkylene) and CH₂CH₂ (C₂alkylene).

22. The compound of claim 21, wherein L is selected from CH₂CH₂CH₂.

23. The compound of claim 20, wherein L is selected from CHCHCH₂, and CH₂CHCH.

24. The compound of any one of claims 1 to 23, wherein A is C₃₋₈heterocycloalkyl including at least one ring heteromoiety selected from N, NH and N(C₁₋₄alkyl), and optionally substituted with one or two substituents selected from OH, F, Cl, C₁₋₂alkyl, C₁₋₂fluoroalkyl, C₁₋₂chloroalkyl, OC₁₋₂alkyl, OC₁₋₂fluoroalkyl and OC₁₋₂chloroalkyl.

25. The compound of claim 24, wherein A is C₃₋₈heterocycloalkyl including at least one ring heteromoiety selected from N, NH and N(C₁₋₄alkyl), and optionally substituted with one or two substituents selected from OH, F, Cl, C₁₋₂alkyl, CF₃, CHF₂, CCl₃, CCl₂H, OCH₃, OCH₂CH₃, OCF₃, OCHF₂, OCH₂CHF₂, OCH₂CF₃, OCH₂CFH₂, OCCl₃, OCH₂CClH₂, OCCl₂H, OCH₂CCl₂H and OCH₂CCl₃.

26. The compound of claim 25, wherein A is selected from aziridinyl, azetidinyl, pyrrolidinyl, morpholinyl, piperazinyl, and piperidinyl and optionally substituted with one or two OH, F, Cl, C₁₋₂alkyl, CF₃, CHF₂, CCl₃, CCl₂H, OCH₃, OCH₂CH₃, OCF₃, OCHF₂, OCH₂CHF₂, OCH₂CF₃, OCH₂CFH₂, OCCl₃, OCH₂CClH₂, OCCl₂H, OCH₂CCl₂H and OCH₂CCl₃.

27. The compound of claim 26, wherein A is selected from pyrrolidinyl, morpholinyl, piperazinyl and piperidinyl and optionally substituted with one or two substituents selected from F, Cl, CH₃, CF₃, OCH₃ and OCF₃.

28. The compound of claim 27, wherein A is selected from pyrrolidinyl, morpholinyl, piperazinyl and piperidinyl.

29. The compound of claim 28, wherein A is piperidinyl.

30. The compound of claim 1, wherein the compound of Formula I is a compound of Formula I-A or a pharmaceutically acceptable salt, solvate and/or prodrug thereof: 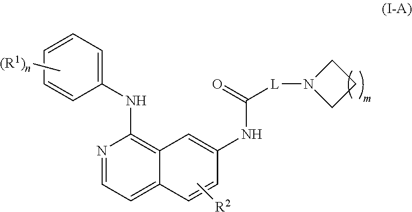
wherein
R¹, R², L and n are as defined for Formula I in any one of claims 1 to 21; and
m is an integer selected from 1 to 3.

31. The compound of claim 1, wherein compound of Formula I is selected from the compounds listed below:

| Compound | | |
|---|---|---|
| I.D | Chemical Name | Structure |
| 1-1 | N-(1-((3-chloro-4- fluorophenyl)amino)-6- methoxyisoquinolin-7-yl)-4- (piperidin-1-yl)butanamide | 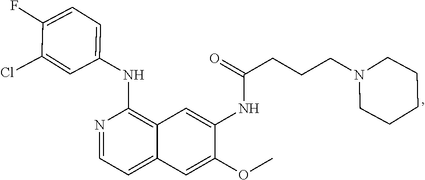 [SMILES: COc1cc2ccnc(Nc3ccc(F)c(Cl)c3)c2cc1NC(=O)CCCN1CCCCC1] |
| 1-2 | N-(1-((3-chloro-4- fluorophenyl)amino)-6- methoxyisoquinolin-7-yl)-5- (piperidin-1-yl)pentanamide | 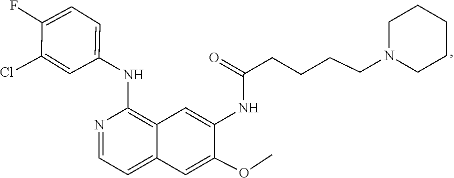 [SMILES: COc1cc2ccnc(Nc3ccc(F)c(Cl)c3)c2cc1NC(=O)CCCCN1CCCCC1] |
| 1-3 | N-(1-((3-chloro-4- fluorophenyl)amino)-6- methoxyisoquinolin-7-yl)-3- (piperidin-1-yl)propanamide | 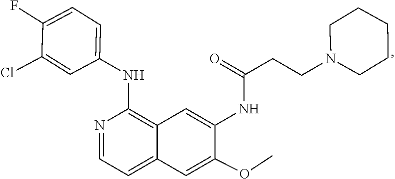 [SMILES: COc1cc2ccnc(Nc3ccc(F)c(Cl)c3)c2cc1NC(=O)CCN1CCCCC1] |
| 1-4 | N-(1-((3-chloro-4- fluorophenyl)amino)-6- methoxyisoquinolin-7-yl)-3- (piperidin-1-yl)propanamide | 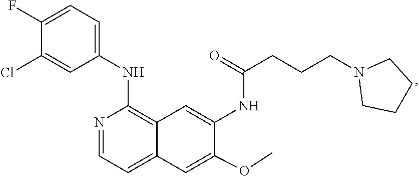 [SMILES: COc1cc2ccnc(Nc3ccc(F)c(Cl)c3)c2cc1NC(=O)CCCN1CCCC1] |
| 1-5 | N-(1-((3-chloro-4- fluorophenyl)amino)-6- methoxyisoquinolin-7-yl)-4- ((2S,6R)-2,6- dimethylmorpholino) butanamide | 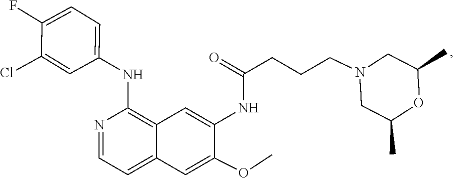 [SMILES: COc1cc2ccnc(Nc3ccc(F)c(Cl)c3)c2cc1NC(=O)CCCN1C[C@@H](C)O[C@@H](C)C1] |
| 1-6 | N-(1-((3-chloro-4- fluorophenyl)amino)-6- methoxyisoquinolin-7-yl)-4- morpholinobutanamide | 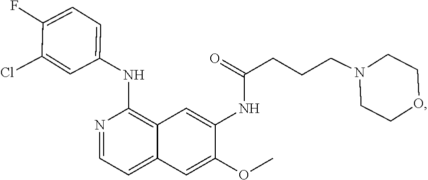 [SMILES: COc1cc2ccnc(Nc3ccc(F)c(Cl)c3)c2cc1NC(=O)CCCN1CCOCC1] |
| 1-7 | N-(1-((3-chloro-4- fluorophenyl)amino)-6- methoxyisoquinolin-7-yl)-4- (4-methylpiperazin-1- yl)butanamide | 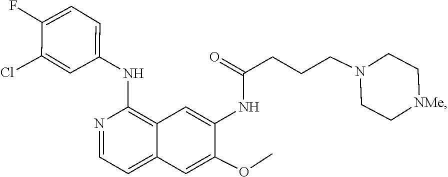 [SMILES: COc1cc2ccnc(Nc3ccc(F)c(Cl)c3)c2cc1NC(=O)CCCN1CCN(C)CC1] |
| 1-8 | N-(1-((3-chloro-4- fluorophenyl)amino)-6- methoxyisoquinolin-7-yl)-4- (4,4-difluoropiperidin-1- yl)butanamide | 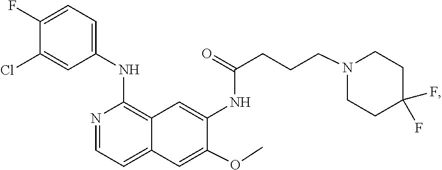 [SMILES: COc1cc2ccnc(Nc3ccc(F)c(Cl)c3)c2cc1NC(=O)CCCN1CCC(F)(F)CC1] |
| 1-9 | N-(1-((3- fluorophenyl)amino)-6- methoxyisoquinolin-7-yl)-4- (piperidin-1-yl)butanamide | 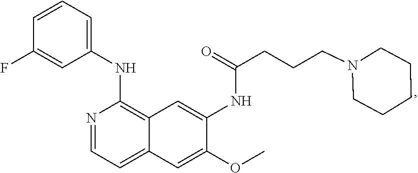 [SMILES: COc1cc2ccnc(Nc3cccc(F)c3)c2cc1NC(=O)CCCN1CCCCC1] |
| 1-10 | N-(6-methoxy-1-((3- methoxyphenyl)amino) isoquinolin-7-yl)-4-(piperidin- 1-yl)butanamide | 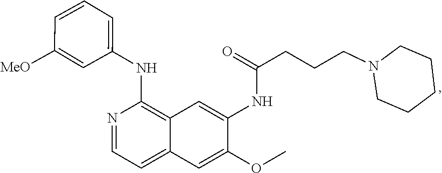 [SMILES: COc1cccc(Nc2nccc3cc(OC)c(NC(=O)CCCN4CCCCC4)cc23)c1] |
| 1-11 | N-(6-methoxy-1-(m- tolylamino)isoquinolin-7-yl)- 4-(piperidin-1-yl)butanamide | 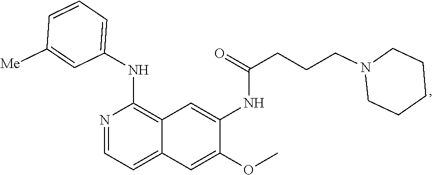 [SMILES: COc1cc2ccnc(Nc3cccc(C)c3)c2cc1NC(=O)CCCN1CCCCC1] |
| 1-12 | N-(1-((4- fluorophenyl)amino)-6- methoxyisoquinolin-7-yl)-4- (piperidin-1-yl)butanamide | 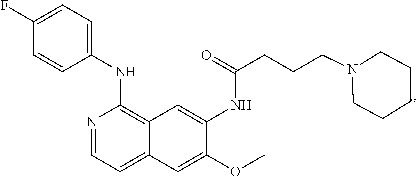 [SMILES: COc1cc2ccnc(Nc3ccc(F)cc3)c2cc1NC(=O)CCCN1CCCCC1] |
| 1-13 | N-(1-((3- chlorophenyl)amino)-6- methoxyisoquinolin-7-yl)-4- (piperidin-1-yl)butanamide | 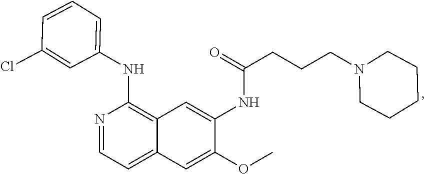 [SMILES: COc1cc2ccnc(Nc3cccc(Cl)c3)c2cc1NC(=O)CCCN1CCCCC1] |
| 1-14 | N-(6-methoxy-1-(p- tolylamino)isoquinolin-7-yl)- 4-(piperidin-1-yl)butanamide | 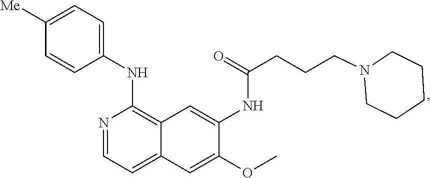 [SMILES: COc1cc2ccnc(Nc3ccc(C)cc3)c2cc1NC(=O)CCCN1CCCCC1] |
| 1-15 | N-(6-methoxy-1- (phenylamino)isoquinolin-7- yl)-4-(piperidin-1- yl)butanamide | 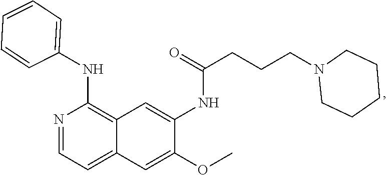 [SMILES: COc1cc2ccnc(Nc3ccccc3)c2cc1NC(=O)CCCN1CCCCC1] |
| 1-16 | N-(1-((4-fluoro-3- (trifluoromethyl)phenyl)amino)- 6-methoxyisoquinolin-7- yl)-4-(piperidin-1- yl)butanamide | 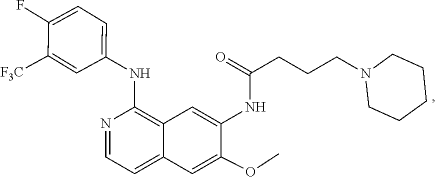 [SMILES: COc1cc2ccnc(Nc3ccc(F)c(C(F)(F)F)c3)c2cc1NC(=O)CCCN1CCCCC1] |
| 1-17 | N-(1-((3-chloro-4- fluorophenyl)amino)-6- methylisoquinolin-7-yl)-4- (piperidin-1-yl)butanamide | 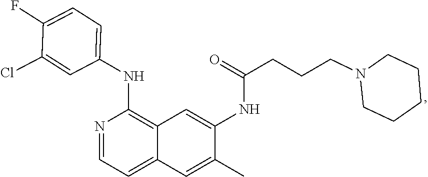 [SMILES: Cc1cc2ccnc(Nc3ccc(F)c(Cl)c3)c2cc1NC(=O)CCCN1CCCCC1] |
| 1-18 | N-(1-((3-chloro-4- fluorophenyl)amino)isoquinolin- 7-yl)-4-(piperidin-1- yl)butanamide | 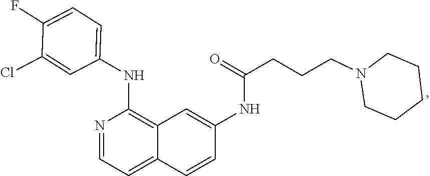 [SMILES: O=C(CCCN1CCCCC1)Nc1ccc2ccnc(Nc3ccc(F)c(Cl)c3)c2c1] |
| 1-19 | N-(1-((2-chloro-4- fluorophenyl)amino)-6- methoxyisoquinolin-7-yl)-4- (piperidin-1-yl)butanamide | 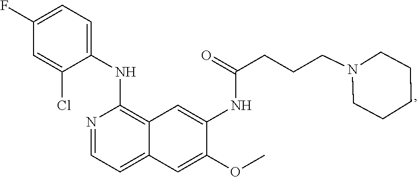 [SMILES: COc1cc2ccnc(Nc3ccc(F)cc3Cl)c2cc1NC(=O)CCCN1CCCCC1] |
| 1-20 | N-(1-((4-fluoro-3- methylphenyl)amino)-6- methoxyisoquinolin-7-yl)-4- (piperidin-1-yl)butanamide | 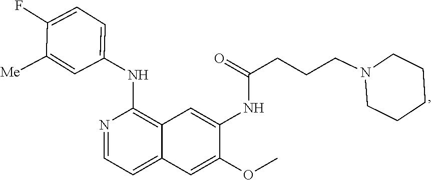 [SMILES: COc1cc2ccnc(Nc3ccc(F)c(C)c3)c2cc1NC(=O)CCCN1CCCCC1] |
| 1-21 | N-(1-((3-chloro-4- methylphenyl)amino)-6- methoxyisoquinolin-7-yl)-4- (piperidin-1-yl)butanamide | 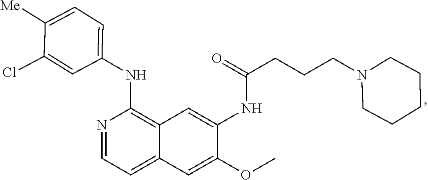 [SMILES: COc1cc2ccnc(Nc3ccc(C)c(Cl)c3)c2cc1NC(=O)CCCN1CCCCC1] |
| 1-22 | N-(1-((4-fluoro-3- methoxyphenyl)amino)-6- methoxyisoquinolin-7-yl)-4- (piperidin-1-yl)butanamide | 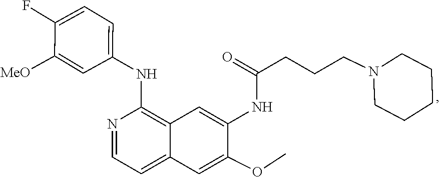 [SMILES: COc1cc(Nc2nccc3cc(OC)c(NC(=O)CCCN4CCCCC4)cc23)ccc1F] |
| 1-23 | N-(1-((3,5-dichloro-4- fluorophenyl)amino)-6- methoxyisoquinolin-7-yl)-4- (piperidin-1-yl)butanamide | 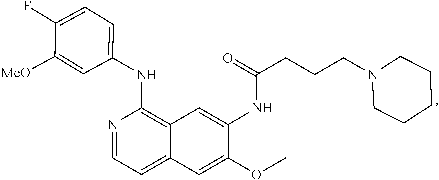 [SMILES: COc1cc(Nc2nccc3cc(OC)c(NC(=O)CCCN4CCCCC4)cc23)ccc1F] |
| 1-24 | N-(1-((3-chloro-4- methoxyphenyl)amino)-6- methoxyisoquinolin-7-yl)-4- (piperidin-1-yl)butanamide | 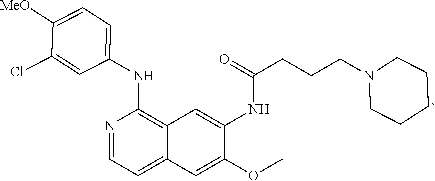 [SMILES: COc1ccc(Nc2nccc3cc(OC)c(NC(=O)CCCN4CCCCC4)cc23)cc1Cl] |
| 1-25 | N-(1-((3-chloro-5- fluorophenyl)amino)-6- methoxyisoquinolin-7-yl)-4- (piperidin-1-yl)butanamide | 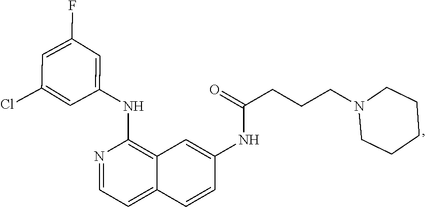 [SMILES: O=C(CCCN1CCCCC1)Nc1ccc2ccnc(Nc3cc(F)cc(Cl)c3)c2c1] |
| 1-26 | N-(6-chloro-1-((3-chloro-4- fluorophenyl)amino) isoquinolin-7-yl)-4-(piperidin- 1-yl)butanamide | 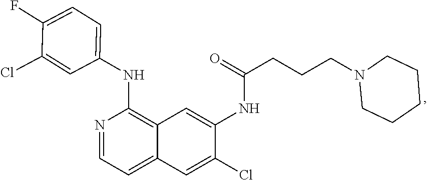 [SMILES: O=C(CCCN1CCCCC1)Nc1cc2c(Nc3ccc(F)c(Cl)c3)nccc2cc1Cl] |
| 1-27 | N-(1-((3,4- dichlorophenyl)amino)-6- methoxyisoquinolin-7-yl)-4- (piperidin-1-yl)butanamide | 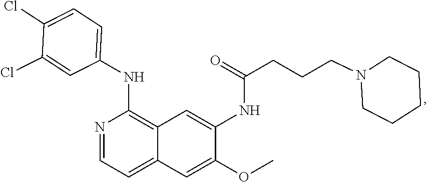 [SMILES: COc1cc2ccnc(Nc3ccc(Cl)c(Cl)c3)c2cc1NC(=O)CCCN1CCCCC1] |
| 1-28 | N-(1-((3-ethyl-4- fluorophenyl)amino)-6- methoxyisoquinolin-7-yl)-4- (piperidin-1-yl)butanamide | 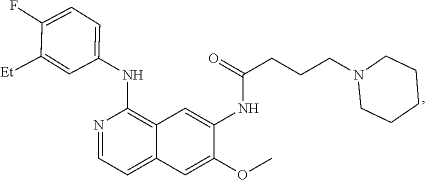 [SMILES: CCc1cc(Nc2nccc3cc(OC)c(NC(=O)CCCN4CCCCC4)cc23)ccc1F] |
| 1-29 | N-(1-((3-chloro-4- fluorophenyl)amino)-5- methoxyisoquinolin-7-yl)-4- (piperidin-1-yl)butanamide | 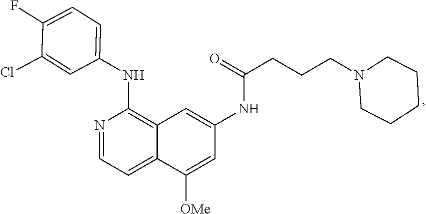 [SMILES: COc1cc(NC(=O)CCCN2CCCCC2)cc2c(Nc3ccc(F)c(Cl)c3)nccc12] |
| 1-30 | N-(1-((4-fluoro-3- isopropylphenyl)amino)-6- methoxyisoquinolin-7-yl)-4- (piperidin-1-yl)butanamide | 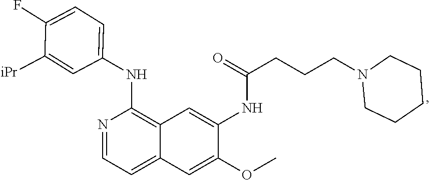 [SMILES: COc1cc2ccnc(Nc3ccc(F)c(C(C)C)c3)c2cc1NC(=O)CCCN1CCCCC1] |
| 1-31 | N-(1-((3-chloro-4- fluorophenyl)amino)-6- methoxyisoquinolin-7-yl)-N- methyl-4-(piperidin-1- yl)butanamide | 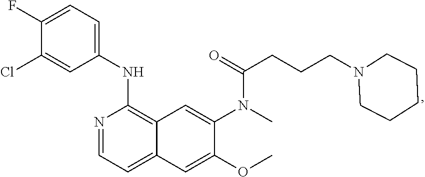 [SMILES: COc1cc2ccnc(Nc3ccc(F)c(Cl)c3)c2cc1N(C)C(=O)CCCN1CCCCC1] |
| 1-32 | N-(1-((3-chloro-4- methylphenyl)amino) isoquinolin-7-yl)-4-(piperidin- 1-yl)butanamide | 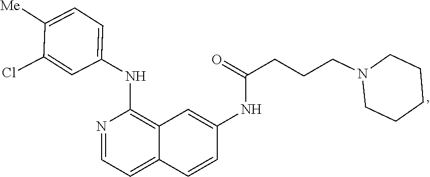 [SMILES: Cc1ccc(Nc2nccc3ccc(NC(=O)CCCN4CCCCC4)cc23)cc1Cl] |
| 1-33 | N-(1-((4-fluoro-3- (trifluoromethyl)phenyl) amino)isoquinolin-7-yl)-4- (piperidin-1-yl)butanamide | 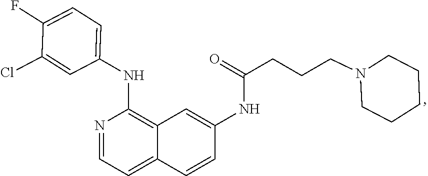 [SMILES: O=C(CCCN1CCCCC1)Nc1ccc2ccnc(Nc3ccc(F)c(Cl)c3)c2c1] |
| 1-34 | N-(1-((3,4-dichloro-2- fluorophenyl)amino) isoquinolin-7-yl)-4-(piperidin- 1-yl)butanamide | 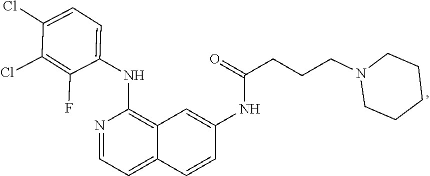 [SMILES: O=C(CCCN1CCCCC1)Nc1ccc2ccnc(Nc3ccc(Cl)c(Cl)c3F)c2c1] |
| 1-35 | N-(1-((3,4- dichlorophenyl)amino) isoquinolin-7-yl)-4-(piperidin- 1-yl)butanamide | 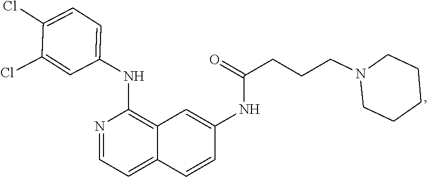 [SMILES: O=C(CCCN1CCCCC1)Nc1ccc2ccnc(Nc3ccc(Cl)c(Cl)c3)c2c1] |
| 1-36 | N-(1-((3,5-dichloro-4- fluorophenyl)amino)isoquinolin- 7-yl)-4-(piperidin-1- yl)butanamide | 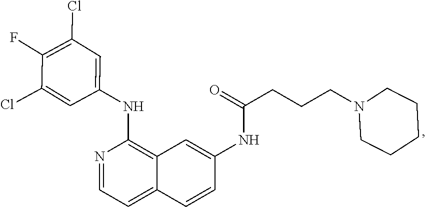 [SMILES: O=C(CCCN1CCCCC1)Nc1ccc2ccnc(Nc3cc(Cl)c(F)c(Cl)c3)c2c1] |
| 1-37 | N-(1-((3,4-dichloro-5- fluorophenyl)amino)isoquinolin- 7-yl)-4-(piperidin-1- yl)butanamide | 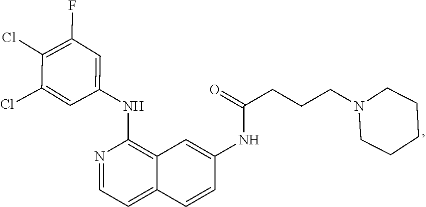 [SMILES: O=C(CCCN1CCCCC1)Nc1ccc2ccnc(Nc3cc(F)c(Cl)c(Cl)c3)c2c1] |
| 1-38 | N-(1-((4,5-dichloro-2- fluorophenyl)amino)isoquinolin- 7-yl)-4-(piperidin-1- yl)butanamide | 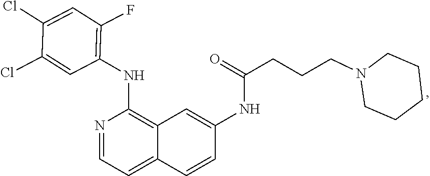 [SMILES: O=C(CCCN1CCCCC1)Nc1ccc2ccnc(Nc3cc(Cl)c(Cl)cc3F)c2c1] |
| 1-39 | N-(1-((3-chloro-2- fluorophenyl)amino)isoquinolin- 7-yl)-4-(piperidin-1- yl)butanamide | 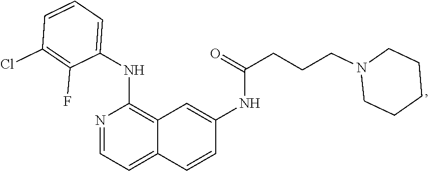 [SMILES: O=C(CCCN1CCCCC1)Nc1ccc2ccnc(Nc3cccc(Cl)c3F)c2c1] |
| 1-40 | N-(1-((3-chloro-2,4- difluorophenyl)amino) isoquinolin-7-yl)-4-(piperidin- 1-yl)butanamide | 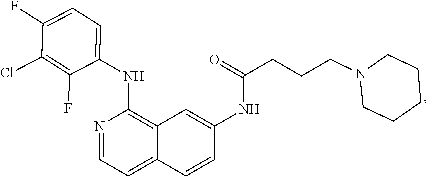 [SMILES: O=C(CCCN1CCCCC1)Nc1ccc2ccnc(Nc3ccc(F)c(Cl)c3F)c2c1] |
| 1-41 | N-(1-((3- cyanophenyl)amino) isoquinolin-7-yl)-4-(piperidin- 1-yl)butanamide | 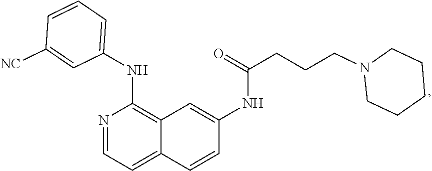 [SMILES: N#Cc1cccc(Nc2nccc3ccc(NC(=O)CCCN4CCCCC4)cc23)c1] |
| 1-42 | N-(1-((3-chloro-4- fluorophenyl)amino)-6- methoxyisoquinolin-7-yl)-3- (pyrrolidin-1-yl)propanamide | 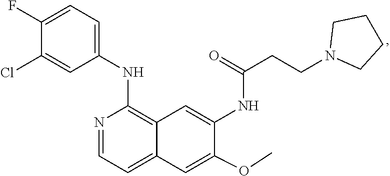 [SMILES: COc1cc2ccnc(Nc3ccc(F)c(Cl)c3)c2cc1NC(=O)CCN1CCCC1] |
| 1-43 | N-(1-((3-chloro-4- fluorophenyl)amino)-6- methoxyisoquinolin-7-yl)-4- (3,3-difluoropyrrolidin-1- yl)butanamide | 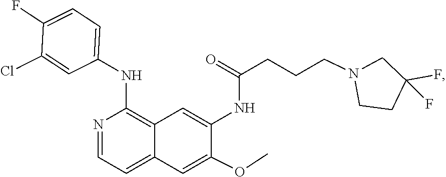 [SMILES: COc1cc2ccnc(Nc3ccc(F)c(Cl)c3)c2cc1NC(=O)CCCN1CCC(F)(F)C1] |
| 1-44 | N-(1-((3,5-dichloro-2- fluorophenyl)amino) isoquinolin-7-yl)-4-(piperidin- 1-yl)butanamide | 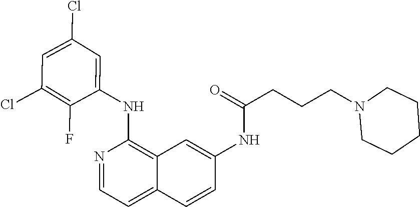 [SMILES: O=C(CCCN1CCCCC1)Nc1ccc2ccnc(Nc3cc(Cl)cc(Cl)c3F)c2c1] |
| 1-45 | N-(1-((4-chloro-2,3- difluorophenyl)amino) isoquinolin-7-yl)-4-(piperidin- 1-yl)butanamide | 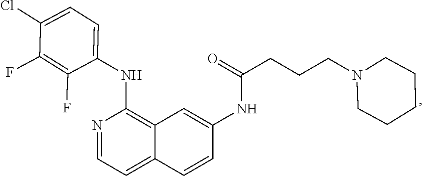 [SMILES: O=C(CCCN1CCCCC1)Nc1ccc2ccnc(Nc3ccc(Cl)c(F)c3F)c2c1] |
| 1-46 | N-(1-((3-chloro-4- fluorophenyl)amino)-6- ethoxyisoquinolin-7-yl)-4- (piperidin-1-yl)butanamide | 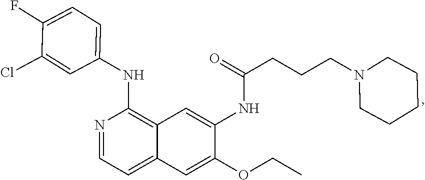 [SMILES: CCOc1cc2ccnc(Nc3ccc(F)c(Cl)c3)c2cc1NC(=O)CCCN1CCCCC1] |
| (R) I-47 | (R)-N-(1-((3-chloro-4- fluorophenyl)amino)-6- methoxyisoquinolin-7-yl)-4- (3-methoxypyrrolidin-1- yl)butanamide | 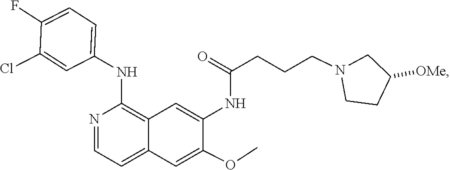 [SMILES: COc1cc2ccnc(Nc3ccc(F)c(Cl)c3)c2cc1NC(=O)CCCN1CC[C@@H](OC)C1] |
| (S) 1-47 | (S)-N-(1-((3-chloro-4- fluorophenyl)amino)-6- methoxyisoquinolin-7-yl)-4- (3-methoxypyrrolidin-1- yl)butanamide | 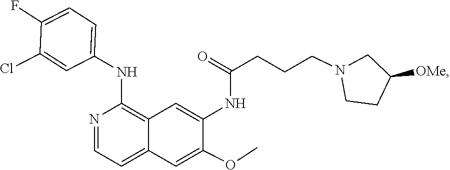 [SMILES: COc1cc2ccnc(Nc3ccc(F)c(Cl)c3)c2cc1NC(=O)CCCN1CC[C@H](OC)C1] |
| (R) 1-48 | (R)-N-(1-((3-chloro-4- fluorophenyl)amino)-6- methoxyisoquinolin-7-yl)-4- (3-fluoropyrrolidin-1- yl)butanamide | 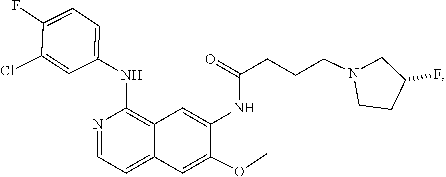 [SMILES: COc1cc2ccnc(Nc3ccc(F)c(Cl)c3)c2cc1NC(=O)CCCN1CC[C@@H](F)C1] |
| (S) I-48 | (S)-N-(1-((3-chloro-4- fluorophenyl)amino)-6- methoxyisoquinolin-7-yl)-4- (3-fluoropyrrolidin-1- yl)butanamide | 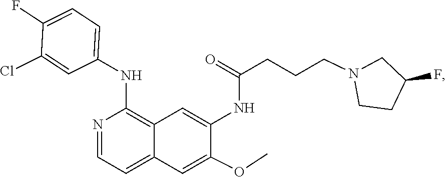 [SMILES: COc1cc2ccnc(Nc3ccc(F)c(Cl)c3)c2cc1NC(=O)CCCN1CC[C@H](F)C1] |
| 1-49 | N-(1-((3-chloro-4- fluorophenyl)amino)-6- propoxyisoquinolin-7-yl)-4- (piperidin-1-yl)butanamide | 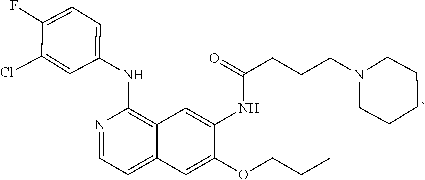 [SMILES: CCCOc1cc2ccnc(Nc3ccc(F)c(Cl)c3)c2cc1NC(=O)CCCN1CCCCC1] |
| 1-50 | N-(1-((3-chloro-4- fluorophenyl)amino)-6- fluoroisoquinolin-7-yl)-4- (piperidin-1-yl)butanamide | 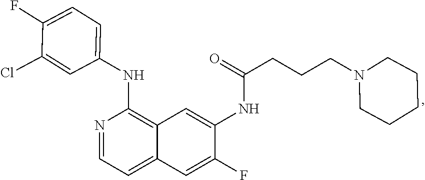 [SMILES: O=C(CCCN1CCCCC1)Nc1cc2c(Nc3ccc(F)c(Cl)c3)nccc2cc1F] |
| 1-51 | N-(1-((3-chloro-4- fluorophenyl)amino)-5- fluoroisoquinolin-7-yl)-4- (piperidin-1-yl)butanamide | 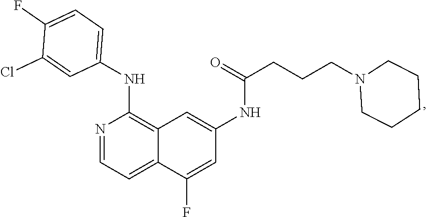 [SMILES: O=C(CCCN1CCCCC1)Nc1cc(F)c2ccnc(Nc3ccc(F)c(Cl)c3)c2c1] |
| 1-52 | (E)-N-(1-((3-chloro-4- fluorophenyl)amino) isoquinolin-7-yl)-4-(piperidin- 1-yl)but-2-enamide | 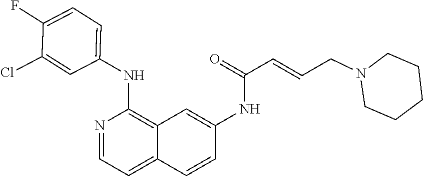 [SMILES: O=C(/C=C/CN1CCCCC1)Nc1ccc2ccnc(Nc3ccc(F)c(Cl)c3)c2c1] |
| | | and |
| 1-53 | N-(1-((3-chloro-4- fluorophenyl)amino)-4- fluoroisoquinolin-7-yl)-4- (piperidin-1-yl)butanamide | 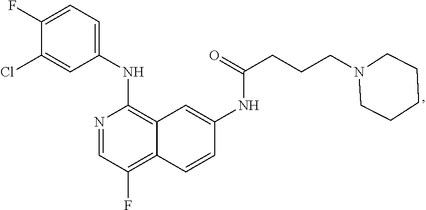 [SMILES: O=C(CCCN1CCCCC1)Nc1ccc2c(F)cnc(Nc3ccc(F)c(Cl)c3)c2c1] |

or a pharmaceutically acceptable salt, solvate and/or prodrug thereof:

32. A pharmaceutical composition comprising one or more compounds of any one of claims 1 to 31 and a pharmaceutically acceptable carrier.

33. A method of inhibiting Bcl2-associated X protein (BAX) and/or Bcl-2 antagonist killer (BAK) in a cell, either in a biological sample or in a patient, comprising administering an effective amount of one or more compounds of any one of claims 1 to 31 to the cell.

34. A method of treating or preventing BAX and/or BAK mediated cell death in a cell either in a biological sample or in a patient comprising administering an effective amount of one or more compounds of any one of claims 1 to 31 to the cell.

35. A method of inhibiting mitochondrial outer membrane permeabilization (MOMP) in a cell, either in a biological sample or in a patient comprising administering an effective amount of one or more compounds of any one of claims 1 to 31 to the cell.

36. A method for inhibiting BAX and BAK oligomerization in a cell, either in a biological sample or in a patient comprising administering an effective amount of one or more compounds of any one of claims 1 to 31 to the cell.

37. A method of treating a disease, disorder or condition that is treatable by inhibiting Bcl2-associated X protein (BAX) and/or Bcl-2 antagonist killer (BAK) comprising administering a therapeutically effective amount of one or more compounds of any one of claims 1 to 31 to a subject in need thereof.

38. The method of claim 37, wherein the disease, disorder or condition that is treatable by inhibiting BAX and/or BAK is a neurodegenerative disease, disorder or condition.

39. The method of claim 37, wherein the disease, disorder or condition that is treatable by inhibiting BAX and/or BAK is neuronal damage associated with ischemia.

40. The method of claim 37, wherein the disease, disorder or condition that is treatable by inhibiting BAX and/or BAK is cardiomyopathy induced by a chemotherapeutic agent.

41. The method of claim 38, wherein the chemotherapeutic agent is doxorubicin.

42. The method of claim 37, wherein the disease, disorder or condition that is treatable by inhibiting BAX and/or BAK is donor hematopoietic stem and progenitor cells (HSPCs) cell death.

43. A method for increasing the survival of donor hematopoietic stem and progenitor cells (HSPCs) during transplantation comprising administering a therapeutically effective amount of one or more compounds of any one of claims 1 to 31 to a subject in need thereof.

44. A method of treating a disease, disorder or condition that is treatable by inhibiting BAX and/or BAK comprising administering a therapeutically effective amount of one or more compounds of any one of claims 1 to 31 in combination with another known agent useful for treatment of a disease, disorder or condition that is treatable by inhibiting BAX and/or BAK to a subject in need thereof.
